(19) United States
(12) Patent Application Publication
(10) Pub. No.: US 2018/0208605 A1
(43) Pub. Date: Jul. 26, 2018

(54) Substituted Aza Compounds as IRAK-4 Inhibitors

(71) Applicant: AURIGENE DISCOVERY TECHNOLOGIES LIMITED, Bangalore (IN)

(72) Inventors: Venkateshwar Rao GUMMADI, Bangalore (IN); Susanta SAMAJDAR, Bangalore (IN); Subhendu MUKHERJEE, Hooghly (IN); Mark Gary BOCK, Boston, MA (US)

(21) Appl. No.: 15/744,451

(22) PCT Filed: Jul. 15, 2016
(86) PCT No.: PCT/IB2016/054229

§ 371 Date: Jan. 12, 2018

(30) Foreign Application Priority Data
Jul. 15, 2015 (IN) 3631/CHE/2015
Jul. 15, 2015 (IN) 3632/CHE/2015

(51) Int. Cl. C07D 513/04; C07D 498/04; C07D 519/00; A61P 35/00; A61P 29/00

(52) U.S. Cl. CPC C07D 513/04; C07D 498/04; A61P 29/00; A61P 35/00; C07D 519/00

(57) ABSTRACT

The present invention provides substituted aza compounds of formula (I) or (II) and pharmaceutically acceptable salts thereof, and their use to inhibit IRAK-4 and/or for the treatment of diseases or disorders induced by IRAK-4.

### CROSS-REFERENCE TO RELATED APPLICATIONS

This application claims the benefit of Indian provisional applications 3631/CHE/2015 and 3632/CHE/2015, both filed on Jul. 15, 2015, which are hereby incorporated by reference in their entirety.

### FIELD OF THE INVENTION

This invention relates to compounds useful for treatment of cancer and inflammatory diseases associated with Interleukin-1 Receptor Associated Kinase (IRAK) and more particularly compounds that modulate the function of IRAK-4. The invention also provides pharmaceutically acceptable compositions comprising compounds of the present invention and methods of using said compositions in the treatment of diseases associated with IRAK-4.

### BACKGROUND OF THE INVENTION

Interleukin-1 (IL-1) Receptor-Associated Kinase-4 (IRAK-4) is a serine/threonine kinase enzyme that plays an essential role in signal transduction by Toll/IL-1 receptors (TIRs). Diverse IRAK enzymes are key components in the signal transduction pathways mediated by interleukin-1 receptor (IL-1R) and Toll-like receptors (TLRs) (Janssens, S, et al. Mol. Cell. 11(2), 2003, 293-302). There are four members in the mammalian IRAK family: IRAK-1, IRAK-2, IRAK-M and IRAK-4. These proteins are characterized by a typical N-terminal death domain that mediates interaction with MyD88-family adaptor proteins and a centrally located kinase domain. The IRAK proteins, as well as MyD88, have been shown to play a role in transducing signals other than those originating from IL-1R receptors, including signals triggered by activation of IL-18 receptors (Kanakaraj, et al. J. Exp. Med. 189(7), 1999, 1129-38) and LPS receptors (Yang, et al., J. Immunol. 163(2), 1999, 639-643). Out of four members in the mammalian IRAK family, IRAK-4 is considered to be the “master IRAK”. Under overexpression conditions, all IRAKs can mediate the activation of nuclear factor-κB (NF-κB) and stress-induced mitogen activated protein kinase (MAPK)-signaling cascades. However, only IRAK-1 and IRAK-4 have been shown to have active kinase activity. While IRAK-1 kinase activity could be dispensable for its function in IL-1-induced NF-κB activation (Kanakaraj et al, J. Exp. Med. 187(12), 1998, 2073-2079) and (Li, et al. Mol. Cell. Biol. 19(7), 1999, 4643-4652), IRAK-4 requires its kinase activity for signal transduction [(Li S, et al. Proc. Natl. Acad. Sci. USA 99(8), 2002, 5567-5572) and (Lye, E et al, J. Biol. Chem. 279(39); 2004, 40653-8)]. Given the central role of IRAK-4 in Toll-like/IL-1R signalling and immunological protection, IRAK4 inhibitors have been implicated as valuable therapeutics in inflammatory diseases, sepsis and autoimmune disorders (Wietek C, et al, Mol., Interv. 2, 2002, 212-215).

Mice lacking IRAK-4 are viable and show complete abrogation of inflammatory cytokine production in response to IL-1, IL-18 or LPS (Suzuki et al. Nature, 416(6882), 2002, 750-756). Similarly, human patients lacking IRAK-4 are severely immunocompromised and are not responsive to these cytokines (Medvedev et al. J. Exp. Med., 198(4), 2003, 521-531 and Picard et al. Science 299(5615), 2003, 2076-2079). Knock-in mice containing inactive IRAK-4 were completely resistant to lipopolysaccharide- and CpG-induced shock (Kim T W, et al. J. Exp. Med 204(5), 2007, 1025-36) and (Kawagoe T, et al. J. Exp. Med. 204 (5), 2007, 1013-1024) and illustrated that IRAK-4 kinase activity is essential for cytokine production, activation of MAPKs and induction of NF-κB regulated genes in response to TLR ligands (Koziczak-Holbro M, et al. J. Biol. Chem. 282 (18), 2007, 13552-13560). Inactivation of IRAK-4 kinase (IRAK-4 KI) in mice leads to resistance to EAE due to reduction in infiltrating inflammatory cells into CNS and reduced antigen specific CD4+ T-cell mediated IL-17 production (Staschke et al., J. Immunol. 183(1), 2009, 568-577).

The crystal structures revealed that IRAK-4 contains characteristic structural features of both serine/threonine and tyrosine kinases, as well as additional novel attributes, including the unique tyrosine gatekeeper residue. Structural analysis of IRAK-4 revealed the underlying similarity with kinase family; ATP-binding cleft sandwiched between bilobal arrangements. The N-terminal lobe consists of mainly of a twisted five-stranded antiparallel beta-sheet and one alpha-helix, and the larger C-terminal lobe is predominantly alpha-helical. Yet, the structure reveals a few unique features for IRAK-4 kinase, including an additional alpha-helix from the N-terminal extension in the N-terminal lobe, a longer loop between helices alpha-D and alpha-E, and a significantly moved helix alpha G as well as its adjoining loops. The ATP-binding site in IRAK-4 has no deep pocket in the back but has a featured front pocket. This uniquely shaped binding pocket provides an excellent opportunity for designing IRAK-4 inhibitors.

The development of IRAK-4 kinase inhibitors has generated several novel classes of protein binders which includes thiazole and pyridine amides (George M Buckley, et al., Bioorg. Med. Chem. Lett., 18(11), 2008, 3211-3214), aminobenzimidazoles (Powers J P, et al., Bioorg. Med. Chem. Lett., 16(11), 2006, 2842-2845), Imidazo[1,2-a] pyridines (Buckley G M, et al., Bioorg. Med. Chem. Lett. 18(12), 2008, 3656-3660) and (Buckley G M, et al. Bioorg. Med. Chem. Lett. 18(11), 2008, 3291-3295), imidazo[1,2-b]pyridazines and benzimidazole-indazoles (WO2008030579 and WO2008030584). Apparently, all of them are still in the early preclinical stage.

Despite various disclosures on different kinase inhibitors, however, with the rise in number of patients affected by kinase enzyme mediated diseases, there appears to be unmet need for newer drugs that can treat such diseases more effectively. There is still need for newer kinase inhibitors including multikinase inhibitors, which may be further useful in treatment of disorders owing to variations in various kinases activity and possessing broader role. They may also be useful as part of other therapeutic regimens for the treatment of disorders, alone or in combination with protein kinase compounds well known by one skilled in the art.

### SUMMARY OF THE INVENTION

In one aspect, the present invention provides a compound of formula (I),

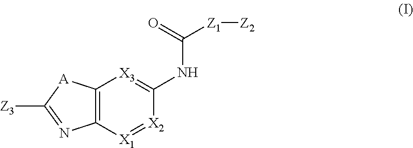

or a pharmaceutically acceptable salt or a stereoisomer thereof;
wherein

each X₁, X₂ and X₃ are independently CR² or N;

A is O, S, S(O) or S(O)₂;

Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;

Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};

Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};

each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;

each R′, R″ and R′″ is independently selected from hydrogen, alkyl, hydroxy, hydroxyalkyl, acyl and cycloalkyl;

each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl)alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;

each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or

Rₐ and R_{b} are taken together along with the atoms which they are attached to form a 3 to 8 membered optionally substituted ring; and

each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

In another aspect, the present invention provides a compound of formula (II),

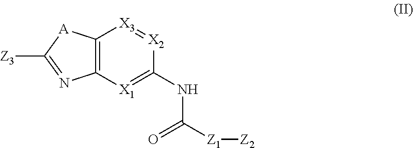

or a pharmaceutically acceptable salt or a stereoisomer thereof;

wherein

each X₁, X₂ and X₃ are independently CR² or N;

A is O, S, S(O) or S(O)₂;

Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;

Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};

Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};

each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;

each R′, R″ and R′″ is independently selected from hydrogen, alkyl, hydroxy, hydroxyalkyl, acyl and cycloalkyl;

each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl)alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;

each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or

Rₐ and R_{b} are taken together along with the atoms which they are attached to form a 3 to 8 membered optionally substituted ring; and

each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

In yet another aspect, the present invention provides a pharmaceutical composition comprising the compound of formula (I) or (II) or a pharmaceutically acceptable salt or a stereoisomer thereof, and at least one pharmaceutically acceptable excipient (such as a pharmaceutically acceptable carrier or diluent).

In yet further aspect, the present invention provides a use of a compound of formula (I) or (II) or a pharmaceutically acceptable salt or a stereoisomer thereof for the treatment or prevention of a disease or a disorder mediated by IRAK-4 enzyme.

More particularly, the invention relates to the use of a compound of formula (I) or (II) or a pharmaceutically acceptable salt or a stereoisomer thereof including mixtures thereof in any ratio as a medicament, for inhibiting IRAK, IRAK-4, or other related kinases.

The compound of formula (I) or (II) of the present invention possesses the therapeutic role of inhibiting IRAK-1 or IRAK-4-related kinases, which are useful in the treatment of diseases and/or disorders including, but not limited to, cancers, allergic diseases and/or disorders, autoimmune diseases and/or disorders, inflammatory diseases and/or disorder and/or conditions associated with inflammation and pain, proliferative diseases, hematopoietic disorders, hematological malignancies, bone disorders, fibrosis diseases and/or disorders, metabolic disorders and/or diseases, muscle diseases and/or disorders respiratory diseases and/or disorders, pulmonary disorders, genetic developmental diseases and/or disorders, neurological and neurodegenerative diseases and/or disorders, chronic inflammatory demyelinating neuropathies, cardiovascular, vascular or heart diseases and/or disorders, ophthalmic/ocular diseases and/or disorders, wound repair, infection and viral diseases. Therefore, inhibition of one or more kinases would have multiple therapeutic indications.

### DETAILED DESCRIPTION OF THE INVENTION

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus it is intended that the present invention include such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In certain embodiments, the present invention provides compounds of formula (I):

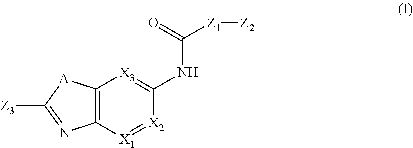

or a pharmaceutically acceptable salt or a stereoisomer thereof;
wherein

each X₁, X₂ and X₃ are independently CR² or N;

A is O, S, S(O) or S(O)₂;

Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;

Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};

Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};

each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;

each R′, R″ and R′″ is independently selected from hydrogen, alkyl, hydroxy, hydroxyalkyl, acyl and cycloalkyl;

each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl) alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;

each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or

Rₐ and R_{b} are taken together along with the atoms which they are attached to form a 3 to 8 membered optionally substituted ring; and

each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

In certain embodiments, the present invention provides compounds of formula (II):

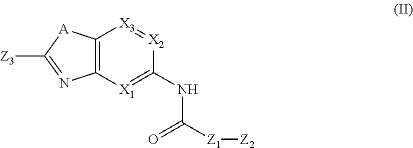

or a pharmaceutically acceptable salt or a stereoisomer thereof;

wherein

each X₁, X₂ and X₃ are independently CR² or N;

A is O, S, S(O) or S(O)₂;

Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;

Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};

Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};

each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;

each R′, R″ and R′″ is independently selected from hydrogen, alkyl, hydroxy, hydroxyalkyl, acyl and cycloalkyl;

each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl)alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;

each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or

Rₐ and R_{b} are taken together along with the atoms which they are attached to form a 3 to 8 membered optionally substituted ring; and

each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

In certain embodiments, the present invention provides the compound of formula (I) or (II)

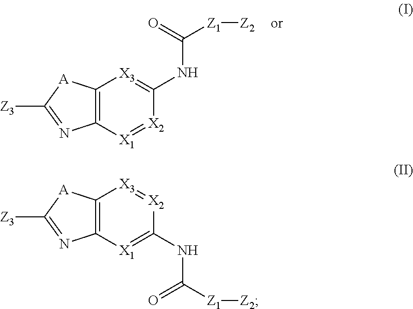

wherein,

X₁, X₂ and X₃ independently are CR² or N;

A is O, S, S(O) or S(O)₂;

Z₁ is optionally substituted monocyclic heteroaryl or optionally substituted monocyclic heterocycloalkyl;

Z₂ is optionally substituted cycloalkyl, optionally substituted aryl, optionally substituted heterocycloalkyl or optionally substituted heteroaryl;

Z₃ is optionally substituted cycloalkyl, optionally substituted aryl, optionally substituted heterocycloalkyl, optionally substituted heteroaryl, optionally substituted aryloxy, optionally substituted heteroaryloxy, optionally substituted cycloalkoxy, optionally substituted (cycloalkyl)alkyl, optionally substituted aralkyl, optionally substituted (heterocycloalkyl)alkyl, optionally substituted heteroaralkyl, optionally substituted (cycloalkyl)-NH—, optionally substituted (cycloalkyl)alkyl-NH—, optionally substituted aralkyl-NH—, optionally substituted (heterocycloalkyl)alkyl-NH—, optionally substituted heteroaralkyl-NH—, optionally substituted (cycloalkyl)alkyl-O—, optionally substituted aralkyl-O—, optionally substituted (heterocycloalkyl)alkyl-O— or optionally substituted heteroaralkyl-O—;

R², independently for each occurrence, is hydrogen, halo, cyano, optionally substituted alkoxy, optionally substituted alkyl, optionally substituted cycloalkyl, optionally substituted (cycloalkyl)alkyl, optionally substituted cycloalkyloxy, optionally substituted aryl, optionally substituted aralkyl, optionally substituted heterocycloalkyl, optionally substituted heteroaryl, optionally substituted (heterocycloalkyl)alkyl, optionally substituted heteroaralkyl, or —NRₐR_{b};

Rₐ and R_{b} are independently hydrogen, alkyl, aminoalkyl, acyl or heterocycloalkyl; or Rₐ and R_{b} are taken together to form an optionally substituted ring.

In certain embodiments, the present invention provides the compound of formula (III) or (IV):

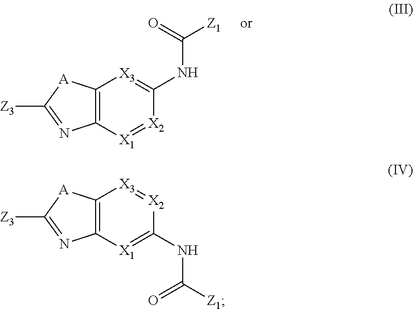

or a pharmaceutically acceptable salt or a stereoisomer thereof;

wherein,

X₁, X₂ and X₃ independently are CR² or N;

A is O, S, S(O) or S(O)₂;

Z₁ is optionally substituted bicyclic heteroaryl or optionally substituted bicyclic heterocycloalkyl;

Z₃ is optionally substituted cycloalkyl, optionally substituted aryl, optionally substituted heterocycloalkyl, optionally substituted heteroaryl, optionally substituted aryloxy, optionally substituted heteroaryloxy, optionally substituted cycloalkoxy, optionally substituted (cycloalkyl)alkyl, optionally substituted aralkyl, optionally substituted (heterocycloalkyl)alkyl, optionally substituted heteroaralkyl, optionally substituted (cycloalkyl)-NH—, optionally substituted (cycloalkyl)alkyl-NH—, optionally substituted aralkyl-NH—, optionally substituted (heterocycloalkyl)alkyl-NH—, optionally substituted heteroaralkyl-NH—, optionally substituted (cycloalkyl)alkyl-O—, optionally substituted aralkyl-O—, optionally substituted (heterocycloalkyl)alkyl-O— or optionally substituted heteroaralkyl-O—;

R², independently for each occurrence, is hydrogen, halo, cyano, optionally substituted alkoxy, optionally substituted alkyl, optionally substituted cycloalkyl, optionally substituted cycloalkoxy, optionally substituted (cycloalkyl)alkyl, optionally substituted aryl, optionally substituted aralkyl, optionally substituted heterocycloalkyl, optionally substituted heteroaryl, optionally substituted (heterocycloalkyl)alkyl, optionally substituted heteroaralkyl, or —NRₐR_{b};

Rₐ and R_{b} are independently hydrogen, alkyl, aminoalkyl, acyl or heterocyclyl; or Rₐ and R_{b} are taken together to form an optionally substituted ring.

In certain embodiments,

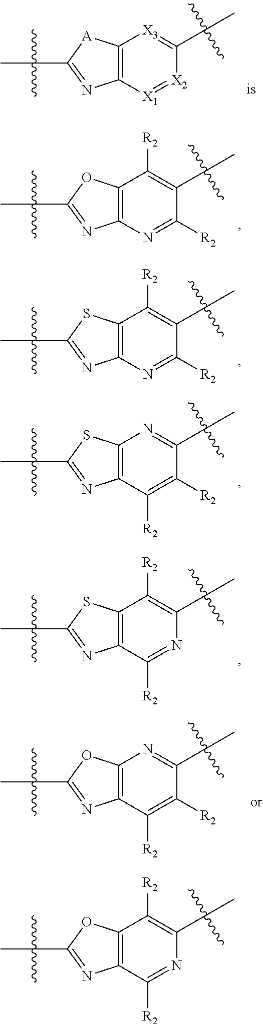

wherein [symbol] is a point of attachment and R₂ is as defined in formula (I).

In certain embodiments,

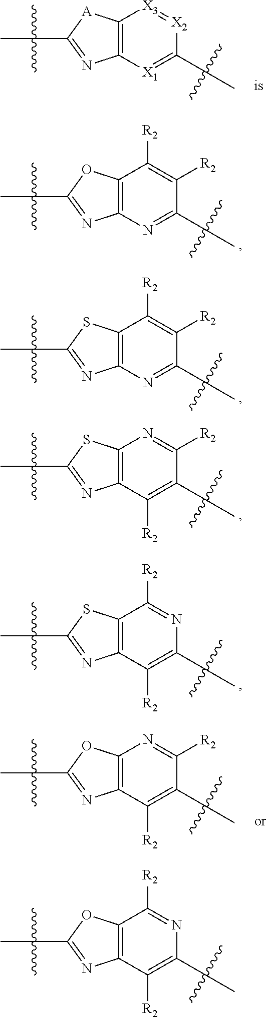

wherein [symbol] is a point of attachment and R₂ is as defined in formula (II).

In certain embodiments, Z₁ is an optionally substituted heteroaryl, optionally substituted heterocycloalkyl, optionally substituted aryl or optionally substituted cycloalkyl. In certain such embodiments, each optional substituent independently represents an occurrence of Rₓ; and Rₓ is as defined for formula (I) or (II).

In certain embodiments, Z₁ is an optionally substituted heteroaryl or optionally substituted heterocycloalkyl; particularly, Z₁ is optionally substituted monocyclic heteroaryl or optionally substituted monocyclic heterocycloalkyl. In certain such embodiments, each optional substituent independently represents an occurrence of Rₓ; and Rₓ is as defined for formula (I) or (II).

In certain embodiments, Z₁ is an optionally substituted heteroaryl or optionally substituted heterocycloalkyl; particularly, Z₁ is optionally substituted bicyclic heteroaryl or optionally substituted bicyclic heterocycloalkyl. In certain such embodiments, each optional substituent independently represents an occurrence of Rₓ; and Rₓ is as defined for formula (I) or (II).

In certain embodiments, Z₁ is an optionally substituted monocyclic heterocycloalkyl.

In certain embodiments, Z₁ is monocyclic heteroaryl or monocyclic heterocycloalkyl; and is substituted by one or more Rₓ; wherein each occurrence of Rₓ is selected from alkyl, alkenyl, alkynyl, halo, hydroxy, haloalkyl, hydroxyalkyl, aminoalkyl, alkoxy, —SH, —S(alkyl), cyano, amido, —C(O)OH, carboxylate, ester, thioester, —C(O)O(alkyl), —C(O)NH(alkyl), oxo, cycloalkyl, cycloalkyloxy, (cycloalkyl)alkyl, aryl, aralkyl, heterocycloalkyl, heteroaryl, (heterocycloalkyl)alkyl-, heteroaralkyl, —NRₐR_{b}, —O—R₄ or —S—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl or haloalkoxy; wherein Rₐ, R_{b} and R₄ are as defined for formula (I) or (II).

In certain embodiments, Z₁ is monocyclic heteroaryl or monocyclic heterocycloalkyl, and is substituted by one or more Rₓ; wherein each occurrence of Rₓ is selected from alkyl, alkenyl, alkynyl, alkoxy, halo, hydroxy, haloalkyl, —NRₐR_{b}, cyano, —C(O)OH, —C(O)O(alkyl), —OC(O)(alkyl), —C(O)NH₂, —C(O)NH(alkyl), cycloalkyl, heterocycloalkyl, (cycloalkyl)alkyl, (heterocycloalkyl)alkyl, cycloalkyl-O—, heterocycloalkyl-O—, (cycloalkyl)alkyl-O—, (heterocycloalkyl)alkyl-O—, aryl, heteroaryl, aralkyl, heteroaralkyl, aryloxy, heteroaryloxy, aralkyl-O—, and heteroaralkyl-O—, any of which is optionally further substituted.

In certain embodiments, Z₁ is monocyclic heteroaryl or monocyclic heterocycloalkyl, substituted by one or more substituents independently selected from —NRₐR_{b} and optionally substituted heterocycloalkyl.

In accordance with any of the foregoing embodiments, Z₁ is furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, or triazinyl; wherein each heteroaryl ring is optionally substituted. In certain such embodiments, each optional substituent independently represents an occurrence of Rₓ; and Rₓ is as defined for formula (I) or (II).

In certain embodiments, Z₁ is an optionally substituted bicyclic heterocycloalkyl.

In certain embodiments, Z₁ is an optionally substituted bicyclic heteroaryl.

In certain embodiments, Z₁ is an optionally substituted bicyclic heterocycloalkyl or optionally substituted bicyclic heteroaryl. In certain such embodiments, each optional substituent independently represents an occurrence of Rₓ, and Rₓ is selected from alkyl, alkenyl, alkynyl, halo, hydroxy, haloalkyl, hydroxyalkyl, aminoalkyl, alkoxy, —SH, —S(alkyl), cyano, amido, —C(O)OH, carboxylate, ester, thioester, —C(O)O(alkyl), —C(O)NH(alkyl), oxo, cycloalkyl, cycloalkyloxy, (cycloalkyl)alkyl, aryl, aralkyl, heterocycloalkyl, heteroaryl, (heterocycloalkyl)alkyl-, heteroaralkyl, —NRₐR_{b}, —O—R₄ or —S—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl or haloalkoxy; wherein Rₐ, R_{b} and R₄ are as defined for formula (I) or (II).

In certain embodiments, Z₁ is optionally substituted bicyclic heteroaryl or optionally substituted bicyclic heterocycloalkyl; wherein the substituent is one, two or three Rₓ; wherein Rₓ is alkyl, alkenyl, alkynyl, alkoxy, halo, hydroxy, haloalkyl, —NRₐR_{b}, cyano, —C(O)OH, —C(O)O(alkyl), —OC(O)(alkyl), —C(O)NH₂, —C(O)NH(alkyl), cycloalkyl, heterocycloalkyl, (cycloalkyl)alkyl, (heterocycloalkyl)alkyl, cycloalkyl-O—, heterocycloalkyl-O—, (cycloalkyl)alkyl-O—, (heterocycloalkyl)alkyl-O—, aryl, heteroaryl, aralkyl, heteroaralkyl, aryloxy, heteroaryloxy, aralkyl-O—, or heteroaralkyl-O—, any of which is optionally further substituted. In certain embodiments, further substitutents are selected from alkyl, halo, haloalkyl, hydroxy, hydroxyalkyl, alkoxy, alkoxyalkyl, amino, nitro, cycloalkyl, (cycloalkyl)alkyl, aryl, aralkyl, heterocycloalkyl, (heterocycloalkyl)alkyl, heteroaryl or (heteroaryl)alkyl.

In certain embodiments, Z₁ is bicyclic heteroaryl or bicyclic heterocycloalkyl, substituted by one or more Rₓ, independently selected from —NRₐR_{b} and optionally substituted heterocycloalkyl.

In certain embodiments, Z₁ is bicyclic heteroaryl, which is substituted by one or more Rₓ; wherein each occurrence of Rₓ is selected from alkyl, alkenyl, alkynyl, alkoxy, halo, hydroxy, haloalkyl, —NRₐR_{b}, cyano, —C(O)OH, —C(O)O(alkyl), —OC(O)(alkyl), amido, —C(O)NH(alkyl), cycloalkyl, heterocycloalkyl, (cycloalkyl)alkyl, (heterocycloalkyl)alkyl, cycloalkyl-O—, heterocycloalkyl-O—, (cycloalkyl)alkyl-O—, (heterocycloalkyl)alkyl-O—, aryl, heteroaryl, aralkyl, heteroaralkyl, aryloxy, heteroaryloxy, aralkyl-O—, and heteroaralkyl-O—, any of which is optionally further substituted. In certain embodiments, further substitutents are selected from alkyl, halo, haloalkyl, hydroxy, hydroxyalkyl, alkoxy, alkoxyalkyl, amino, nitro, cycloalkyl, (cycloalkyl)alkyl, aryl, aralkyl, heterocycloalkyl, (heterocycloalkyl)alkyl, heteroaryl or (heteroaryl)alkyl.

In certain embodiments, Z₁ is benzimidazolyl, benzoxadiazolyl, benzoxathiadiazolyl, cinnolinyl, furopyridinyl, naphthyridinyl, quinolinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, purinyl, benzotriazolyl, benzotriadiazolyl, carbazolyl, dibenzothienyl, acridinyl and pyrazolopyrimidyl; each of which is optionally substituted. In certain such embodiments, each optional substituent independently represents an occurrence of Rₓ; and Rₓ is as defined for formula (I) or (II).

In certain embodiments, Z₁ is selected from phenyl, naphthyl, furanyl, thienyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl, pyridazinyl, benzimidazolyl, benzoxadiazolyl, benzoxathiadiazolyl, cinnolinyl, furopyridinyl, naphthyridinyl, quinolinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, purinyl, benzotriazolyl, benzotriadiazolyl, carbazolyl, dibenzothienyl, acridinyl, pyrazolopyrimidyl, azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl or azabicyclo[13.2]octanyl; each of which is optionally substituted, and each substituent independently represents an occurrence of Rₓ; and Rₓ is as defined in formula (I) or (II).

In certain embodiments, Z₁ is optionally substituted oxazolyl, optionally substituted pyridyl, optionally substituted furanyl, optionally substituted pyrimidiyl, optionally substituted pyrazinyl, optionally substituted imadazolyl or optionally substituted pyrrolopyrimdyl. In some embodiments, each optional substituent on Z₁ is independently selected from alkyl, alkenyl, alkynyl, halo, hydroxy, haloalkyl, hydroxyalkyl, aminoalkyl, alkoxy, —SH, —S(alkyl), cyano, amido, —C(O)OH, carboxylate, ester, thioester, —C(O)O(alkyl), —C(O)NH(alkyl), oxo, cycloalkyl, cycloalkyloxy, (cycloalkyl)alkyl, aryl, aralkyl, heterocycloalkyl, heteroaryl, (heterocycloalkyl)alkyl-, heteroaralkyl, —NRₐR_{b}, —O—R₄ and —S—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl and haloalkoxy.

In certain embodiments, Z₂ is optionally substituted heterocycloalkyl or optionally substituted heteroaryl. In certain such embodiments, each optional substituent independently represents an occurrence of R_{y}; and R_{y} is as defined for formula (I) or (II).

In certain embodiments, Z₂ is absent.

In certain embodiments, Z₂ is heterocycloalkyl or heteroaryl which is substituted with one or more R_{y}, wherein each occurrence of R_{y} is selected from —NRₐR_{b}, optionally substituted heterocycloalkyl and optionally substituted heteroaryl.

In certain embodiments, Z₂ is optionally substituted pyridyl; In certain such embodiments, each optional substituent is independently selected from alkyl, halo, hydroxy, haloalkyl, hydroxyalkyl, aminoalkyl, alkoxy, cyano, amido, carboxylic acid, carboxylate, ester, alkoxycarbonyl, oxo, cycloalkyl, aryl, aralkyl, heterocycloalkyl, heteroaryl, (heterocycloalkyl)alkyl-, heteroaralkyl, —NRₐR_{b}, and —O—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl and haloalkoxy; wherein Rₐ, R_{b}, and R₄ are as defined for formula (I) or (II).

In certain embodiments, Z₂ is optionally substituted pyrrolidinyl. In certain such embodiments, each optional substituent is independently selected from alkyl, halo, hydroxy, haloalkyl, hydroxyalkyl, aminoalkyl, alkoxy, cyano, amido, carboxylic acid, carboxylate, ester, alkoxycarbonyl, oxo, cycloalkyl, aryl, aralkyl, heterocycloalkyl, heteroaryl, (heterocycloalkyl)alkyl-, heteroaralkyl, —NRₐR_{b}, and —O—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl and haloalkoxy; wherein Rₐ, R_{b}, and R₄ are as defined for formula (I) or (II).

In certain embodiments, Z₂ is optionally substituted oxazolyl or optionally substituted imadazolyl. In certain such embodiments, each optional substituent is independently selected from alkyl, halo, hydroxy, haloalkyl, hydroxyalkyl, aminoalkyl, alkoxy, cyano, amido, carboxylic acid, carboxylate, ester, alkoxycarbonyl, oxo, cycloalkyl, aryl, aralkyl, heterocycloalkyl, heteroaryl, (heterocycloalkyl)alkyl-, heteroaralkyl, —NRₐR_{b}, and —O—R₄; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl and haloalkoxy; wherein Rₐ, R_{b}, and R₄ are as defined for formula (I) or (II).

In certain embodiments, Z₃ is optionally substituted cycloalkyl, optionally substituted aryl, optionally substituted heterocycloalkyl or optionally substituted heteroaryl. In certain such embodiments, each optional substituent independently represents an occurrence of R_{z}; and R_{z} is as defined for formula (I) or (II).

In certain embodiments, Z₃ is optionally substituted heterocycloalkyl.

In certain embodiments, Z₃ is heterocycloalkyl optionally substituted with alkyl, alkenyl, alkynyl, alkoxy, halo, hydroxy, haloalkyl, —NRₐR_{b}, cyano, —C(O)OH, —C(O)O(alkyl), —OC(O)(alkyl), —C(O)NH₂, or —C(O)NH(alkyl).

In certain embodiments, Z₃ is heterocycloalkyl, which is optionally substituted with one or more R_{z}, wherein each occurrence of R_{z} is selected from alkyl, alkenyl, alkynyl, alkoxy, halo, hydroxy, haloalkyl, —NRₐR_{b}, cyano, —C(O)OH, —C(O)O(alkyl), —OC(O)(alkyl), —C(O)NH₂, and —C(O)NH(alkyl).

In certain embodiments, Z₃ is optionally substituted cycloalkyl, optionally substituted aryl, optionally substituted heterocycloalkyl, optionally substituted heteroaryl, optionally substituted aryloxy, optionally substituted heteroaryloxy, optionally substituted cycloalkoxy, optionally substituted (cycloalkyl)alkyl, optionally substituted aralkyl, optionally substituted (heterocycloalkyl)alkyl, optionally substituted heteroaralkyl, optionally substituted (cycloalkyl)-NH—, optionally substituted (cycloalkyl)alkyl-NH—, optionally substituted aralkyl-NH—, optionally substituted (heterocycloalkyl)alkyl-NH—, optionally substituted heteroaralkyl-NH—, optionally substituted (cycloalkyl)alkyl-O—, optionally substituted aralkyl-O—, optionally substituted (heterocycloalkyl)alkyl-O— or optionally substituted heteroaralkyl-O—. In certain such embodiments, each optional substituent independently represents an occurrence of R_{z}; and R_{z} is as defined for formula (I) or (II).

In certain embodiments, Z₃ is optionally substituted cycloalkyl, optionally substituted aryl, optionally substituted heterocycloalkyl or optionally substituted heteroaryl. In certain such embodiments, each optional substituent independently represents an occurrence of R_{z}; and R_{z} is as defined for formula (I) or (II).

In certain embodiments, Z₃ is optionally substituted heterocycloalkyl. In certain such embodiments, each optional substituent independently represents an occurrence of R_{z}; and R_{z} is as defined for formula (I) or (II).

In certain embodiments, Z₃ is heterocycloalkyl, which is optionally substituted with one or more R_{z}, wherein each occurrence of R_{z} is selected from alkyl, alkenyl, alkynyl, alkoxy, halo, hydroxy, haloalkyl, cyano, —C(O)OH, —C(O)(alkyl)-OH, —C(O)O(alkyl), —OC(O)(alkyl), —C(O)NH₂, and —C(O)NH(alkyl).

In certain embodiments, each R² is independently hydrogen, optionally substituted heterocycloalkyl, or optionally substituted heteroaryl. In certain such embodiments, at least one occurrence of R² is heterocycloalkyl substituted by hydroxyl, hydroxyalkyl, or a combination thereof.

In certain embodiments, at least one occurrence of R² is optionally substituted piperidinyl or pyrrolidinyl. In certain such embodiments, at least one occurrence of R² is piperidinyl or pyrrolidinyl, substituted by hydroxyl, hydroxyalkyl, or a combination thereof.

In certain embodiments, at least one occurrence of R² is optionally substituted pyridyl. In certain such embodiments, each optional substituent independently represents an occurrence of R_{z}, and R_{z} is as defined for formula (I) or (II).

In certain embodiments, at least one occurrence of R² is optionally substituted cycloalkyl. In certain preferred embodiments, at least one occurrence of R² is optionally substituted cyclopropyl, optionally substituted cyclobutyl, optionally substituted cyclopentyl or optionally substituted cyclohexyl. In certain such embodiments, each optional substituent is independently selected from alkyl, alkoxy, halo, haloalkyl, hydroxy, hydroxyalkyl, —SH, —S(alkyl), cyano, amido, amino, carboxylate and oxo.

In accordance with any of the foregoing embodiments, R² is optionally substituted cycloalkyloxy. In certain such embodiments, at least one occurrence of R² is cycloalkyloxy, substituted by heterocycloalkyl or heteroaryl.

In certain embodiments, X₁, X₂ and X₃ independently are CR² or N; provided that at least one of X₁, X₂ and X₃ is N.

In certain embodiments, X₁ is N; X₂ and X₃ independently are CR² or N;

In certain embodiments, A is O or S.

In certain embodiments, R² is optionally substituted heterocycloalkyl. In certain such embodiments, each optional substituent is independently selected from hydrogen, hydroxy, hydroxyalkyl, halo, alkyl and oxo.

In certain embodiments, R² is alkyl or haloalkyl.

In accordance with any of the foregoing embodiments, R² is optionally substituted heteroaryl.

In accordance with any of the foregoing embodiments, R² is optionally substituted cycloalkyl.

In certain embodiments, at least one occurrence of R² is haloalkyl, optionally substituted alkyl, optionally substituted cycloalkyl, optionally substituted aryl, optionally substituted heterocycloalkyl, optionally substituted heteroaryl, —NRₐR_{b}, —O—R₃ or —S—R₃; wherein each optional substituent is independently selected from alkyl, alkoxy, halo, haloalkyl, hydroxy, hydroxyalkyl, amido, amino, carboxylate, oxo and cycloalkyl; wherein Rₐ, R_{b}, and R₃ are as defined in formula (I) or (II).

In accordance with any of the foregoing embodiments, Rₐ and R_{b} are independently hydrogen, alkyl, aminoalkyl, acyl, acylamino or heterocycloalkyl.

In accordance with any of the foregoing embodiments, Rₐ and R_{b} are taken together to form an optionally substituted ring.

In certain embodiments, Rₐ and R_{b} are taken together along with the atoms which they are attached to form a 3 to 8 membered optionally substituted ring;

In certain embodiments, Rₓ is selected from alkyl, halo, hydroxy, haloalkyl, hydroxyalkyl, aminoalkyl, alkoxy, amido, carboxylic acid, carboxylate, —C(O)NH(alkyl), oxo, cycloalkyl, aryl, —NRₐR_{b} and —O—R₄; optionally wherein the cycloalkyl and aryl are further substituted by one or more substituents selected from halo, haloalkyl, amino, hydroxy, alkyl, cyano, aminoalkyl, hydroxyalkyl and haloalkoxy; wherein Rₐ, R_{b}, and R₄ are as defined in formula (I) or (II).

In certain embodiments, R_{y} is selected from alkyl, halo, hydroxy, haloalkyl, hydroxyalkyl, aminoalkyl, alkoxy, amido, carboxylic acid, carboxylate, —C(O)NH(alkyl), oxo, cycloalkyl, aryl, —NRₐR_{b} and —O—R₄; optionally wherein the cycloalkyl and aryl are further substituted by one or more substituents selected from halo, haloalkyl, amino, hydroxy, alkyl, cyano, aminoalkyl, hydroxyalkyl and haloalkoxy; wherein Rₐ, R_{b}, and R₄ are as defined in formula (I) or (II).

In certain embodiments, the compound of formula (I) is a compound of formula (IA)

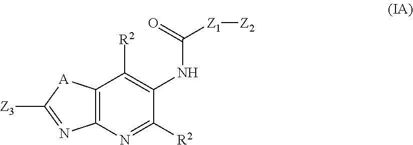

wherein Z₁, Z_{2,} Z₃, A and R² are as defined in compound of formula (I).

In certain embodiments, the compound of formula (I) is a compound of formula (IB)

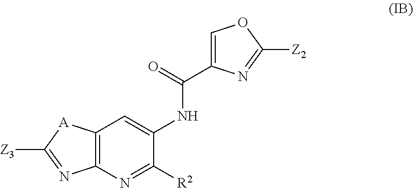

wherein,

Z₂ is optionally substituted 6-membered heteroaryl;

Z₃ is optionally substituted 6-membered heterocycloalkyl; and

A is O or S; and R₂ is as defined in formula (I).

In certain embodiments of the compound of formula (IB), R² is

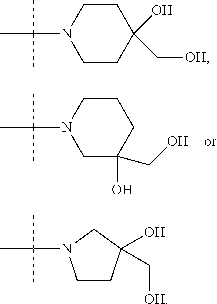

In certain embodiments, the compound of formula (I) is a compound of formula (IC)

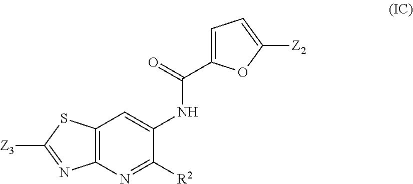

wherein,

Z₂ is optionally substituted 6-membered heteroaryl; and

Z₃ is optionally substituted 6-membered heterocycloalkyl.

In certain embodiments of the compound of formula (IC), R² is

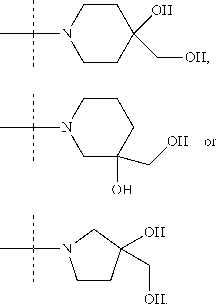

In certain embodiments, the compound of formula (I) is a compound of formula (ID)

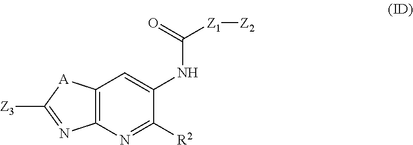

wherein R² is optionally substituted cycloalkyloxy; and

Z₁, Z₂, Z₃ and A are as defined in compound of formula (I).

In certain embodiments, the compound of formula (II) is a compound of formula (IIA)

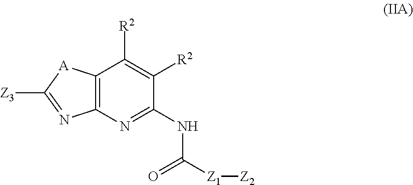

wherein Z₁, Z₂, Z₃, A and R² are as defined in compound of formula (II).

In certain embodiments, the present invention provides a compound or a pharmaceutically acceptable salt or a stereoisomer thereof, selected from:

| Example | IUPAC name |
|---|---|
| 1 | N-(2-(4-methylpiperazin-1-yl)-5-(piperidin-1-yl)thiazolo[4,5-b]pyridin-6-yl)-2- |
| | (2-methylpyridin-4-yl)oxazole-4-carboxamide hydrochloride; |
| 2 | N-(5-(4-hydroxy-4-(hydroxymethyl)piperidin-1-yl)-2-morpholinothiazolo[4,5- |
| | b]pyridin-6-yl)-5-(2-methylpyridin-4-yl)furan-2-carboxamide hydrochloride; |
| 3 | 2-(1-methyl-6-oxo-1,6-dihydropyridin-3-yl)-N-(2-morpholino-5-(piperidin-1- |
| | yl)thiazolo[4,5-b]pyridin-6-yl)oxazole-4-carboxamide; |
| 4 | N-(5-(4-hydroxy-4-(hydroxymethyl)piperidin-1-yl)-2-morpholinothiazolo[4,5- |
| | b]pyridin-6-yl)-2-(2-methylpyridin-4-yl)oxazole-5-carboxamide hydrochloride; |
| 5 | N-(5-(3-hydroxy-3-(hydroxymethyl)piperidin-1-yl)-2-morpholinothiazolo[4,5- |
| | b]pyridin-6-yl)-5-(2-methylpyridin-4-yl)furan-2-carboxamide hydrochloride; |
| 6 | 2-(2-methylpyridin-4-yl)-N-(2-morpholino-5-(((1r,4r)-4- |
| | morpholinocyclohexyl)oxy)thiazolo[4,5-b]pyridin-6-yl)oxazole-4-carboxamide |
| | hydrochloride; |
| 7 | 2-(2-methylpyridin-4-yl)-N-(2-(piperazin-1-yl)-5-(piperidin-1-yl)thiazolo[4,5- |
| | b]pyridin-6-yl)oxazole-4-carboxamide hydrochloride; |
| 8 | (S)-N-(5-(4-hydroxy-4-(hydroxymethyl)piperidin-1-yl)-2- |
| | morpholinothiazolo[4,5-b]pyridin-6-yl)-6-(3-hydroxypyrrolidin-1- |
| | yl)picolinamide hydrochloride; |
| 9 | N-(5-(3-hydroxy-3-(hydroxymethyl)pyrrolidin-1-yl)-2-morpholinothiazolo[4,5- |
| | b]pyridin-6-yl)-2-(2-methylpyridin-4-yl)oxazole-4-carboxamide hydrochloride; |
| 10 | N-(2-(4-(2-hydroxyacetyl)piperazin-1-yl)-5-(piperidin-1-yl)thiazolo[4,5- |
| | b]pyridin-6-yl)-2-(2-methylpyridin-4-yl)oxazole-4-carboxamide hydrochloride; |
| 11 | 2-(6-aminopyridin-2-yl)-N-(2-morpholino-5-(piperidin-1-yl)thiazolo[4,5- |
| | b]pyridin-6-yl)oxazole-4-carboxamide hydrochloride; |
| 12 | 2-(2-amino-5-chloropyridin-4-yl)-N-(2-morpholino-5-(piperidin-1- |
| | yl)thiazolo[4,5-b]pyridin-6-yl)oxazole-4-carboxamide; |
| 13 | 2-(5-fluoro-1-methyl-6-oxo-1,6-dihydropyridin-3-yl)-N-(2-morpholino-5- |
| | (piperidin-1-yl)thiazolo[4,5-b]pyridin-6-yl)oxazole-4-carboxamide; |
| 14 | N-(5-(5-methylpyridin-2-yl)-2-morpholinooxazolo[4,5-b]pyridin-6-yl)-2-(2- |
| | methylpyridin-4-yl)oxazole-4-carboxamide; |
| 15 | N-(5-(3-hydroxy-3-(hydroxymethyl)piperidin-1-yl)-2-morpholinooxazolo[4,5- |
| | b]pyridin-6-yl)-2-(2-methylpyridin-4-yl)oxazole-4-carboxamide; |
| 16 | 2-(2-methylpyridin-4-yl)-N-(2-morpholino-5-(((1r,4r)-4- |
| | morpholinocyclohexyl)oxy)oxazolo[4,5-b]pyridin-6-yl)oxazole-4-carboxamide |
| | hydrochloride; |
| 17 | (S)-N-(5-(3-fluoropyrrolidin-1-yl)-2-morpholinooxazolo[4,5-b]pyridin-6-yl)-2- |
| | (2-methylpyridin-4-yl)oxazole-4-carboxamide; |
| 18 | (R)-N-(5-(3-fluoropyrrolidin-1-yl)-2-morpholinooxazolo[4,5-b]pyridin-6-yl)-2- |
| | (2-methylpyridin-4-yl)oxazole-4-carboxamide hydrochloride; |
| 19 | N-(2-morpholino-5-(piperidin-1-yl)thiazolo[4,5-b]pyridin-6-yl)pyrazolo[1,5- |
| | a]pyrimidine-3-carboxamide; |
| 20 | N-(5-(5-methylpyridin-2-yl)-2-morpholinothiazolo[4,5-b]pyridin-6- |
| | yl)pyrazolo[1,5-a]pyrimidine-3-carboxamide; |
| 21 | 5-(3-aminopiperidin-1-yl)-N-(5-cyclopropyl-2-morpholinothiazolo[4,5-b]pyridin- |
| | 6-yl)pyrazolo[1,5-a]pyrimidine-3-carboxamide; |
| 22 | 5-((2-aminoethyl)amino)-N-(5-cyclopropyl-2-morpholinothiazolo[4,5-b]pyridin- |
| | 6-yl)pyrazolo[1,5-a]pyrimidine-3-carboxamide; |
| 23 | 5-(3-aminopyrrolidin-1-yl)-N-(5-cyclopropyl-2-morpholinothiazolo[4,5- |
| | b]pyridin-6-yl)pyrazolo[1,5-a]pyrimidine-3-carboxamide hydrochloride; |
| 24 | N-(2-morpholino-5-(piperidin-1-yl)oxazolo[4,5-b]pyridin-6-yl)pyrazolo[1,5- |
| | a]pyrimidine-3-carboxamide; |
| 25 | (R)-N-(5-(3-hydroxypyrrolidin-1-yl)-2-morpholinooxazolo[4,5-b]pyridin-6- |
| | yl)pyrazolo[1,5-a]pyrimidine-3-carboxamide; |
| 26 | (S)-6-(3-aminopyrrolidin-1-yl)-N-(2-morpholino-5-(piperidin-1-yl)thiazolo[4,5- |
| | b]pyridin-6-yl)picolinamide; |
| 27 | (S)-6-(3-(2-aminoacetamido)pyrrolidin-1-yl)-N-(2-morpholino-5-(piperidin-1- |
| | yl)thiazolo[4,5-b]pyridin-6-yl)picolinamide; |
| 28 | N-(5-((R)-3-hydroxypiperidin-1-yl)-2-morpholinothiazolo[4,5-b]pyridin-6-yl)-6- |
| | ((S)-3-hydroxypyrrolidin-1-yl)picolinamide; |
| 29 | (S)-6-(3-hydroxypiperidin-1-yl)-N-(2-morpholino-5-(piperidin-1- |
| | yl)thiazolo[4,5-b]pyridin-6-yl)picolinamide; |
| 30 | (R)-6-(3-hydroxypiperidin-1-yl)-N-(2-morpholino-5-(piperidin-1- |
| | yl)thiazolo[4,5-b]pyridin-6-yl)picolinamide; |
| 31 | 6-(4-methoxypiperidin-1-yl)-N-(2-morpholino-5-(piperidin-1-yl)thiazolo[4,5- |
| | b]pyridin-6-yl)picolinamide; |
| 32 | 6-(4-(hydroxymethyl)piperidin-1-yl)-N-(2-morpholino-5-(piperidin-1- |
| | yl)thiazolo[4,5-b]pyridin-6-yl)picolinamide; |
| 33 | (R)-6-(2-(hydroxymethyl)morpholino)-N-(2-morpholino-5-(piperidin-1- |
| | yl)thiazolo[4,5-b]pyridin-6-yl)picolinamide; |
| 34 | (R)-6-(3-hydroxypyrrolidin-1-yl)-N-(2-morpholino-5-(piperidin-1- |
| | yl)thiazolo[4,5-b]pyridin-6-yl)picolinamide; |
| 35 | 6-(4-(2-hydroxyethyl)piperidin-1-yl)-N-(2-morpholino-5-(piperidin-1- |
| | yl)thiazolo[4,5-b]pyridin-6-yl)picolinamide; |
| 36 | (S)-1-(6-((2-morpholino-5-(piperidin-1-yl)thiazolo[4,5-b]pyridin-6- |
| | yl)carbamoyl)pyridin-2-yl)pyrrolidin-3-ylglycinate 2,2,2-trifluoroacetate; |
| 37 | N-(2-morpholino-5-(piperidin-1-yl)thiazolo[4,5-b]pyridin-6-yl)-6-(((tetrahydro- |
| | 2H-pyran-4-yl)methyl)amino)picolinamide; |
| 38 | (R)-6-(4-hydroxypiperidin-1-yl)-N-(5-(3-hydroxypiperidin-1-yl)-2- |
| | morpholinothiazolo[4,5-b]pyridin-6-yl)picolinamide; |
| 39 | (R)-6-(azepan-1-yl)-N-(5-(3-hydroxypiperidin-1-yl)-2-morpholinothiazolo[4,5- |
| | b]pyridin-6-yl)picolinamide; |
| 40 | (R)-N-(5-(3-hydroxypiperidin-1-yl)-2-morpholinothiazolo[4,5-b]pyridin-6-yl)-6- |
| | (piperidin-1-yl)picolinamide; |
| 41 | (R)-N-(5-(3-hydroxypiperidin-1-yl)-2-morpholinothiazolo[4,5-b]pyridin-6-yl)-2′- |
| | methyl-[2,4′-bipyridine]-6-carboxamide; |
| 42 | (S)-6-(3-hydroxypyrrolidin-1-yl)-N-(2-morpholino-5-(piperidin-1- |
| | yl)thiazolo[4,5-b]pyridin-6-yl)picolinamide hydrochloride; |
| 43 | 6-(3-hydroxypyrrolidin-1-yl)-N-(5-methyl-2-morpholinothiazolo[4,5-b]pyridin- |
| | 6-yl)picolinamide; |
| 44 | 6-(3-hydroxypyrrolidin-1-yl)-N-(2-morpholino-5-(trifluoromethyl)thiazolo[4,5- |
| | b]pyridin-6-yl)picolinamide; |
| 45 | 6-(2-oxa-5-azabicyclo[2.2.1]heptan-5-yl)-N-(2-morpholino-5-(piperidin-1- |
| | yl)thiazolo[4,5-b]pyridin-6-yl)picolinamide; |
| 46 | 6-((4-(hydroxymethyl)cyclohexyl)amino)-N-(2-morpholino-5-(piperidin-1- |
| | yl)thiazolo[4,5-b]pyridin-6-yl)picolinamide; |
| 47 | (S)-diethyl (1-(6-((2-morpholino-5-(piperidin-1-yl)thiazolo[4,5-b]pyridin-6- |
| | yl)carbamoyl)pyridin-2-yl)pyrrolidin-3-yl)phosphate; |
| 48 | methyl 1-(6-((2-morpholino-5-(piperidin-1-yl)thiazolo[4,5-b]pyridin-6- |
| | yl)carbamoyl)pyridin-2-yl)-2-oxoimidazolidine-4-carboxylate; |
| 49 | 1-(6-((2-morpholino-5-(piperidin-1-yl)thiazolo[4,5-b]pyridin-6- |
| | yl)carbamoyl)pyridin-2-yl)-2-oxoimidazolidine-4-carboxylic acid; |
| 50 | N-(5-cyclopropyl-2-morpholinothiazolo[4,5-b]pyridin-6-yl)-6-(3- |
| | hydroxypyrrolidin-1-yl)pyrazine-2-carboxamide; |
| 51 | 6′-amino-N-(5-cyclopropyl-2-morpholinothiazolo[4,5-b]pyridin-6-yl)-[2,3′- |
| | bipyridine]-6-carboxamide; |
| 52 | 2′-amino-N-(5-cyclopropyl-2-morpholinothiazolo[4,5-b]pyridin-6-yl)-[2,4′- |
| | bipyridine]-6-carboxamide; |
| 53 | 2-(3-hydroxypyrrolidin-1-yl)-N-(2-morpholino-5-(piperidin-1-yl)thiazolo[4,5- |
| | b]pyridin-6-yl)pyrimidine-4-carboxamide; |
| 54 | 6-(3-hydroxypyrrolidin-1-yl)-N-(2-morpholino-5-(piperidin-1-yl)thiazolo[4,5- |
| | b]pyridin-6-yl)pyrazine-2-carboxamide hydrochloride; |
| 55 | 6-(3-hydroxypiperidin-1-yl)-N-(2-morpholino-5-(piperidin-1-yl)thiazolo[4,5- |
| | b]pyridin-6-yl)picolinamide; |
| 56 | 6-(4-hydroxypiperidin-1-yl)-N-(2-morpholino-5-(piperidin-1-yl)thiazolo[4,5- |
| | b]pyridin-6-yl)picolinamide; |
| 57 | (S)-1-(6-((2-morpholino-5-(piperidin-1-yl)thiazolo[4,5-b]pyridin-6- |
| | yl)carbamoyl)pyridin-2-yl)pyrrolidin-3-ylalaninate; |
| 58 | (S)-ethyl (1-(6-((2-morpholino-5-(piperidin-1-yl)thiazolo[4,5-b]pyridin-6- |
| | yl)carbamoyl)pyridin-2-yl)pyrrolidin-3-yl)carbonate; |
| 59 | (R)-2-(3-hydroxypyrrolidin-1-yl)-N-(2-morpholino-5-(piperidin-1- |
| | yl)thiazolo[4,5-b]pyridin-6-yl)pyrimidine-4-carboxamide; |
| 60 | (S)-2-(3-hydroxypyrrolidin-1-yl)-N-(2-morpholino-5-(piperidin-1- |
| | yl)thiazolo[4,5-b]pyridin-6-yl)pyrimidine-4-carboxamide; |
| 61 | N-(2-morpholino-5-(piperidin-1-yl)thiazolo[4,5-b]pyridin-6-yl)-6-(3- |
| | oxopiperazin-1-yl)picolinamide; |
| 62 | (R)-6-(3-hydroxypyrrolidin-1-yl)-N-(2-morpholino-5-(piperidin-1- |
| | yl)thiazolo[4,5-b]pyridin-6-yl)pyrazine-2-carboxamide; |
| 63 | (S)-6-(3-hydroxypyrrolidin-1-yl)-N-(2-morpholino-5-(piperidin-1- |
| | yl)thiazolo[4,5-b]pyridin-6-yl)pyrazine-2-carboxamide; |
| 64 | (S)-1-(4-((2-morpholino-5-(piperidin-1-yl)thiazolo[4,5-b]pyridin-6- |
| | yl)carbamoyl)pyrimidin-2-yl)pyrrolidin-3-yl glycinate; |
| 65 | (S)-1-(6-((2-morpholino-5-(piperidin-1-yl)thiazolo[4,5-b]pyridin-6- |
| | yl)carbamoyl)pyrazin-2-yl)pyrrolidin-3-yl glycinate; |
| 66 | 1-(6-((2-morpholino-5-(piperidin-1-yl)thiazolo[4,5-b]pyridin-6- |
| | yl)carbamoyl)pyridin-2-yl)piperidin-4-yl glycinate; |
| 67 | 6-(4-(hydroxymethyl)-1H-imidazol-1-yl)-N-(2-morpholino-5-(piperidin-1- |
| | yl)thiazolo[4,5-b]pyridin-6-yl)picolinamide; |
| 68 | (S)-4-(3-hydroxypyrrolidin-1-yl)-N-(2-morpholino-5-(piperidin-1- |
| | yl)thiazolo[4,5-b]pyridin-6-yl)picolinamide; |
| 69 | 6-(4-(hydroxymethyl)-1H-pyrazol-1-yl)-N-(2-morpholino-5-(piperidin-1- |
| | yl)thiazolo[4,5-b]pyridin-6-yl)picolinamide; |
| 70 | 6-(4-(aminomethyl)-1H-imidazol-1-yl)-N-(2-morpholino-5-(piperidin-1- |
| | yl)thiazolo[4,5-b]pyridin-6-yl)picolinamide 2,2,2-trifluoroacetate; |
| 71 | sodium 1-(6-((2-morpholino-5-(piperidin-1-yl)thiazolo[4,5-b]pyridin-6- |
| | yl)carbamoyl)pyridin-2-yl)piperidine-4-carboxylate; |
| 72 | (R)-N-(5-(3-hydroxypyrrolidin-1-yl)-2-morpholinothiazolo[4,5-b]pyridin-6-yl)- |
| | 4-(2-methylpyridin-4-yl)-1H-imidazole-2-carboxamide 2,2,2-trifluoroacetate; |
| 73 | (R)-N-(5-(3-hydroxypyrrolidin-1-yl)-2-morpholinothiazolo[4,5-b]pyridin-6-yl)- |
| | 4-(2-methylpyridin-4-yl)-1H-pyrrole-2-carboxamide; |
| 74 | 2-(5-methoxypyridin-2-yl)-N-(2-morpholino-5-(piperidin-1-yl)thiazolo[4,5- |
| | b]pyridin-6-yl)oxazole-4-carboxamide; |
| 75 | 6′-amino-N-(2-morpholino-5-(piperidin-1-yl)thiazolo[4,5-b]pyridin-6-yl)-[2,3′- |
| | bipyridine]-6-carboxamide; |
| 76 | N-(2,5-dimorpholinothiazolo[4,5-b]pyridin-6-yl)-6-(3-hydroxypyrrolidin-1- |
| | yl)picolinamide; |
| 77 | 6-(3-(hydroxymethyl)pyrrolidin-1-yl)-N-(2-morpholino-5-(piperidin-1- |
| | yl)thiazolo[4,5-b]pyridin-6-yl)picolinamide; |
| 78 | 6-(3-(hydroxymethyl)piperidin-1-yl)-N-(2-morpholino-5-(piperidin-1- |
| | yl)thiazolo[4,5-b]pyridin-6-yl)picolinamide; |
| 79 | 1-(6-((2,5-dimorpholinothiazolo[4,5-b]pyridin-6-yl)carbamoyl)pyridin-2- |
| | yl)pyrrolidin-3-yl glycinate; |
| 80 | 1-(6-((2-morpholino-5-(piperidin-1-yl)thiazolo[4,5-b]pyridin-6- |
| | yl)carbamoyl)pyridin-2-yl)piperidine-4-carboxylic acid; |
| 81 | 6-(3-hydroxy-8-azabicyclo[3.2.1]octan-8-yl)-N-(2-morpholino-5-(piperidin-1- |
| | yl)thiazolo[4,5-b]pyridin-6-yl)picolinamide; |
| 82 | 6-(4-carbamoylpiperidin-1-yl)-N-(2-morpholino-5-(piperidin-1-yl)thiazolo[4,5- |
| | b]pyridin-6-yl)picolinamide; |
| 83 | 2′-amino-N-(2-morpholino-5-(piperidin-1-yl)thiazolo[4,5-b]pyridin-6-yl)-[2,4′- |
| | bipyridine]-6-carboxamide; |
| 84 | (S)-6-(3-hydroxypyrrolidin-1-yl)-5-methyl-N-(2-morpholino-5-(piperidin-1- |
| | yl)thiazolo[4,5-b]pyridin-6-yl)picolinamide; |
| 85 | 6′-amino-3-methyl-N-(2-morpholino-5-(piperidin-1-yl)thiazolo[4,5-b]pyridin-6- |
| | yl)-[2,3′-bipyridine]-6-carboxamide; |
| 86 | N-(5-(4,4-dimethyl-2-oxopyrrolidin-1-yl)-2-morpholinooxazolo[4,5-b]pyridin-6- |
| | yl)-2-(2-methylpyridin-4-yl)oxazole-4-carboxamide; |
| 87 | (R)-2-(6-aminopyridin-2-yl)-N-(5-(3-hydroxypyrrolidin-1-yl)-2- |
| | morpholinooxazolo[4,5-b]pyridin-6-yl)oxazole-4-carboxamide hydrochloride; |
| 88 | (R)-2-(2-amino-5-chloropyridin-4-yl)-N-(5-(3-hydroxypyrrolidin-1-yl)-2- |
| | morpholinooxazolo[4,5-b]pyridin-6-yl)oxazole-4-carboxamide; |
| 89 | 2-(2-methylpyridin-4-yl)-N-(2-morpholino-5-(2-oxopyrrolidin-1-yl)oxazolo[4,5- |
| | b]pyridin-6-yl)oxazole-4-carboxamide hydrochloride; |
| 90 | (R)-6′-amino-N-(5-(3-hydroxypyrrolidin-1-yl)-2-morpholinooxazolo[4,5- |
| | b]pyridin-6-yl)-[2,3′-bipyridine]-6-carboxamide hydrochloride; |
| 91 | (R)-N-(5-(3-hydroxypyrrolidin-1-yl)-2-morpholinooxazolo[4,5-b]pyridin-6-yl)-5- |
| | (2-methylpyridin-4-yl)furan-2-carboxamide hydrochloride; |
| 92 | 6′-amino-N-(2,5-dimorpholinooxazolo[4,5-b]pyridin-6-yl)-[2,3′-bipyridine]-6- |
| | carboxamide hydrochloride; |
| 93 | 2-(6-aminopyridin-2-yl)-N-(2-morpholino-5-(piperidin-1-yl)oxazolo[4,5- |
| | b]pyridin-6-yl)oxazole-4-carboxamide; |
| 94 | 6-(1-((S)-2-hydroxypropyl)-1H-pyrazol-4-yl)-N-(5-((R)-3-hydroxypyrrolidin-1- |
| | yl)-2-morpholinooxazolo[4,5-b]pyridin-6-yl)picolinamide; |
| 95 | (R)-N-(5-(3-(cyclopropylmethoxy)pyrrolidin-1-yl)-2-morpholinooxazolo[4,5- |
| | b]pyridin-6-yl)-2-(2-methylpyridin-4-yl)oxazole-4-carboxamide; |
| 96 | 6′-amino-N-(2-morpholino-5-(piperidin-1-yl)oxazolo[4,5-b]pyridin-6-yl)-[2,3′- |
| | bipyridine]-6-carboxamide; |
| 97 | (R)-1-(6-(2-(2-methylpyridin-4-yl)oxazole-4-carboxamido)-2- |
| | morpholinooxazolo[4,5-b]pyridin-5-yl)pyrrolidin-3-yl glycinate; |
| 98 | 5-(4-aminopiperidin-1-yl)-N-(5-cyclopropyl-2-morpholinothiazolo[4,5-b]pyridin- |
| | 6-yl)pyrazolo[1,5-a]pyrimidine-3-carboxamide 2,2,2-trifluoroacetate; |
| 99 | 5-(4-aminopiperidin-1-yl)-N-(5-cyclopropyl-2-morpholinothiazolo[4,5-b]pyridin- |
| | 6-yl)pyrazolo[1,5-a]pyrimidine-3-carboxamide 2,2,2-trifluoroacetate; |
| 100 | (S)-N-(5-cyclopropyl-2-morpholinothiazolo[4,5-b]pyridin-6-yl)-5-(3- |
| | hydroxypiperidin-1-yl)pyrazolo[1,5-a]pyrimidine-3-carboxamide; |
| 101 | N-(5-cyclopropyl-2-morpholinothiazolo[4,5-b]pyridin-6-yl)-5-(4- |
| | hydroxypiperidin-1-yl)pyrazolo[1,5-a]pyrimidine-3-carboxamide; |
| 102 | 5-(3-aminopiperidin-1-yl)-N-(5-((R)-3-hydroxypyrrolidin-1-yl)-2- |
| | morpholinothiazolo[4,5-b]pyridin-6-yl)pyrazolo[1,5-a]pyrimidine-3-carboxamide |
| | hydrochloride; |
| 103 | (R)-N-(5-(3-hydroxypyrrolidin-1-yl)-2-morpholinothiazolo[4,5-b]pyridin-6- |
| | yl)pyrazolo[1,5-a]pyrimidine-3-carboxamide hydrochloride; |
| 104 | N-(2-morpholino-5-(piperidin-1-yl)thiazolo[4,5-b]pyridin-6-yl)pyrazolo[1,5- |
| | a]pyrimidine-3-carboxamide hydrochloride; |
| 105 | N-(5-cyclopropyl-2-morpholinothiazolo[4,5-b]pyridin-6-yl)pyrazolo[1,5- |
| | a]pyrimidine-3-carboxamide; |
| 106 | N-(5-cyclopropyl-2-morpholinothiazolo[4,5-b]pyridin-6-yl)pyrazolo[1,5- |
| | a]pyrimidine-3-carboxamide hydrochloride; or |
| 107 | N-(5-cyclopropyl-2-morpholinooxazolo[4,5-b]pyridin-6-yl)pyrazolo[1,5- |
| | a]pyrimidine-3-carboxamide; |

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the term “or” refers to “and/or”, unless stated otherwise.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to when the alkyl may be substituted as well as the event or circumstance where the alkyl is not substituted.

The term “substituted” refers to moieties having substituents replacing hydrogen on one or more carbons of the backbone. Thus, a moiety that is optionally substituted may have one or more hydrogens of the indicated moiety be replaced by a substituent, each of which may be the same or different. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, a sulfhydryl, an alkylthio, a sulfate, a sulfonate, a sulfamoyl, a sulfonamido, a sulfonyl, a heterocyclyl, an aralkyl, and an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the term “optionally substituted” refers to the replacement of one to six hydrogen radicals on the same carbon or on different carbons in a given structure with the radical of a specified substituent including, but not limited to: hydroxyl, hydroxyalkyl, alkoxy, alkoxyalkyl, halogen, alkyl, aryl, aryloxy, aralkyl, heteroaryl, heteroaryloxy, heteroaralkyl, cycloalkyl, cycloalkoxy, (cycloalkyl)alkyl, heterocycloalkyl, (heterocycloalkyl)alkyl, amino, aminoalkyl, alkylamino, dialkylamino, acyl, —C(O)₂H, —O(acyl), —NH(acyl), —N(alkyl)(acyl), cyano, phosphinate, phosphate, phosphonate, sulfonate, sulfonamido, sulfate, haloalkyl or haloalkoxy. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including, but not limited to, C₁-C₁₀ straight-chain alkyl groups or C₃-C₁₀ branched-chain alkyl groups. Preferably, the “alkyl” group refers to C₁-C₆ straight-chain alkyl groups or C₃-C₆ branched-chain alkyl groups. Most preferably, the “alkyl” group refers to C₁-C₄ straight-chain alkyl groups or C₃-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl or heteroaryl groups is contemplated.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl or heteroaryl groups is contemplated.

The term “acyl” refers to a group R—CO— wherein R is an optionally substituted alkyl group defined above. Examples of ‘acyl’ groups are, but not limited to, CH₃CO—, CH₃CH₂CO—, CH₃CH₂CH₂CO— or (CH₃)₂CHCO—.

As used herein, the term “alkoxy” refers to alkyl groups (as defined above) bonded to an oxygen atom that is attached to a core structure. Preferably, alkoxy groups have one to six carbon atoms. Examples of alkoxy groups include, but are not limited to, methoxy, ethoxy, propoxy, isopropoxy, butoxy, isobutoxy, tert-butoxy, pentoxy, 3-methyl butoxy and the like.

As used herein, the term “haloalkyl” refers to alkyl group (as defined above) is substituted with one or more halogens. A monohaloalkyl radical, for example, may have a chlorine, bromine, iodine or fluorine atom. Dihalo and polyhaloalkyl radicals may have two and more of the same or different halogen atoms respectively. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like.

As used herein, the term “haloalkoxy” refers to radicals wherein one or more of the hydrogen atoms of the alkoxy group are substituted with one or more halogens. Representative examples of “haloalkoxy” groups include, but are not limited to, difluoromethoxy (—OCHF₂), trifluoromethoxy (—OCF₃) or trifluoroethoxy (—OCH₂CF₃).

As used herein, the term “aryl” alone or in combination with other term(s) means a 6- to 10-membered carbocyclic aromatic system containing one or two rings wherein such rings may be fused. The term “fused” means that the second ring is attached or formed by having two adjacent atoms in common with the first ring. The term “fused” is equivalent to the term “condensed”. Examples of aryl groups include but are not limited to phenyl, naphthyl or indanyl. Unless otherwise specified, all aryl groups described herein may be optionally substituted.

The terms “amine” and “amino” are art-recognized and refer to both unsubstituted and substituted amines and salts thereof, e.g., a moiety that can be represented by

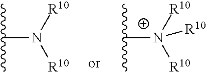

wherein each R¹⁰ independently is a hydrogen or a hydrocarbyl group, or two R¹⁰ are taken together with the N atom to which they are attached to form a heterocycle having from 4 to 8 atoms in the ring structure.

As used herein, “aminoalkyl” refers to an amino group, as defined above, in which one or two hydrogen atoms are substituted with alkyl group. A carbon atom of the alkyl group is attached to the parent molecular group.

As used herein, “nitro” refers to an —NO₂ group.

As used herein, “alkylamino” and “cycloalkylamino”, refer to an —N-group, wherein nitrogen atom of said group being attached to alkyl or cycloalkyl respectively. Representative examples of an “alkylamino” and “cycloalkylamino” groups include, but are not limited to, —NHCH₃ and —NH-cyclopropyl. An amino group can be optionally substituted with one or more of the suitable groups.

As used herein the term “cycloalkyl” alone or in combination with other term(s) means C₃-C₁₀ saturated cyclic hydrocarbon ring. A cycloalkyl may be a single ring, which typically contains from 3 to 7 carbon ring atoms. Examples of single-ring cycloalkyls include cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused, and spirocyclic carbocyclyls.

As used herein, “cycloalkyloxy” refers to an —O-cycloalkyl group wherein the cycloalkyl group is as defined above.

As used herein, the term “cyano” refers to a —CN group.

As used herein, the term “hydroxy” or “hydroxyl” refers to —OH group.

As used herein, the term “oxo” refers to a ═O group.

The term “glycinate”, as used herein, refers to a group —C(O)ONH₂(CH₂).

The term “alaninate”, as used herein, refers to a group —C(O)ONH₂(CH)CH₃.

As used herein, the term “thiol” or “sulfhydryl” refers to a —SH group.

As used herein, the term “hydroxyalkyl” or “hydroxylalkyl” means alkyl substituted with one or more hydroxyl groups, wherein the alkyl groups are as defined above. Examples of “hydroxyalkyl” include, but are not limited to, hydroxymethyl, hydroxyethyl, hydroxypropyl, propan-2-ol and the like.

As used herein, the term “halo” or “halogen” alone or in combination with other term(s) means fluorine, chlorine, bromine or iodine.

The term “carboxylate” refers to a group represented by the formula —(CO₂)⁻.

The term “ester”, as used herein, refers to a group —C(O)OR¹¹ wherein R¹¹ represents a hydrocarbyl group.

The term “thioester”, as used herein, refers to a group —C(O)SR¹¹ or —SC(O)R¹¹ wherein R^{x} represents a hydrocarbyl.

The term “phosphinate”, as used herein, refers to a group —P(O)(OR¹¹)R¹¹ wherein R¹¹ represents a hydrocarbyl group.

The term “phosphate”, as used herein, refers to a group —OP(O)(OR¹¹)₂ wherein R¹¹ represents a hydrocarbyl group.

The term “phosphonate”, as used herein, refers to a group —P(O)(OR¹¹)₂ wherein R¹¹ represents a hydrocarbyl group.

The term “sulfonamido”, as used herein, refers to a group —S(O)₂N(R¹¹)₂ wherein R¹¹ represents a hydrocarbyl group.

As used herein, the term “heterocycloalkyl” refers to a non-aromatic, saturated or partially saturated, monocyclic or polycyclic ring system of 3 to 15 members having at least one heteroatom or heterogroup selected from O, N, S, S(O), S(O)₂, NH and C(O) with the remaining ring atoms being independently selected from the group consisting of carbon, oxygen, nitrogen, and sulfur. The term “heterocycloalkyl” also refers to a bridged bicyclic ring system having at least one heteroatom or heterogroup selected from O, N, S, S(O), S(O)₂, NH or C(O). The “monocyclic heterocycloalkyl” refers to non-aromatic, saturated or partially saturated, monocyclic heterocycloalkyl rings having 4 to 7 member atoms. Examples of “monocyclic heterocycloalkyl” include, but are not limited to, azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl and N-oxides thereof. The “bicyclic heterocycloalkyl” refers to non-aromatic, saturated or partially saturated, monocyclic heterocycloalkyl rings having 7 to 11 member atoms. Examples of “bicyclic heterocycloalkyl” include, but are not limited to, indolinyl, indolinylmethyl, aza-bicyclooctanyl, azocinyl, chromanyl, xanthenyl and N-oxides thereof. Attachment of a heterocycloalkyl substituent can occur via either a carbon atom or a heteroatom. A heterocycloalkyl group can be optionally substituted by one or more aforesaid groups.

Preferably, “heterocycloalkyl” refers to a 5- to 6-membered ring selected from azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocycloalkyl” includes azetidinyl, pyrrolidinyl, morpholinyl and piperidinyl. All heterocycloalkyl groups are optionally substituted by one or more aforesaid groups.

As used herein, the term “heteroaryl” refers to an aromatic heterocyclic ring system containing 5 to 20 ring atoms, preferably 5 to 10 ring atoms, which can be a monocyclic heteroaryl or bicyclic heteroaryl or polycyclic heteroaryl fused together or linked covalently. The rings may contain from 1 to 4 heteroatoms selected from N, O and S, wherein the N or S atom is optionally oxidized or the N atom is optionally quarternized. Any suitable ring position of the heteroaryl moiety may be covalently linked to the parent molecular structure.

The “monocyclic heteroaryl” refers to a 5- or 6-membered heteroaryl ring. The 5 membered ring consists of two double bonds and one, two, three or four heteroatoms selected from N, O and S, wherein the N or S atom is optionally oxidized or the N atom is optionally quarternized. The 6 membered ring consists of three double bonds and one, two, three or four N atoms wherein the N atom is optionally oxidized or quarternized. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl All monocyclic heteroaryls are optionally substituted by one or more aforesaid groups.

As used herein, the term “bicyclic heteroaryl” refers to a monocyclic heteroaryl fused to a phenyl, a monocyclic cycloalkyl, a monocyclic cycloalkenyl, a monocyclic heterocycloalkyl, or a monocyclic heteroaryl. The fused cycloalkyl or heterocycloalkyl portion of the bicyclic heteroaryl group is optionally substituted. When the bicyclic heteroaryl is a monocyclic heteroaryl fused to a phenyl ring, then the bicyclic heteroaryl group is attached to the parent molecular moiety through any carbon atom or nitrogen atom within the bicyclic ring system. When the bicyclic heteroaryl contains a fused cycloalkyl, cycloalkenyl, heteroaryl or heterocycloalkyl, then the bicyclic heteroaryl group is attached to the parent molecular moiety through any carbon or nitrogen atom contained within the monocyclic heteroaryl portion of the bicyclic ring system.

As used herein, the term “heterocyclyl” includes definitions of “heterocycloalkyl” and “heteroaryl”.

As used herein, the term ‘(cycloalkyl)alkyl’, ‘arylalkyl’, ‘(heterocycloalkyl)alkyl’ or ‘heteroaralkyl’ refers to an alkyl group which is further substituted by cycloalkyl, aryl, heterocycloalkyl or heteroaryl respectively, wherein cycloalkyl, aryl, heterocycloalkyl and heteroaryl are as above defined.

As used herein, the term ‘compound(s)’ comprises the compounds disclosed in the present invention.

As used herein, the terms “comprise” and “comprising” are generally used in the sense of include, that is to say permitting the presence of one or more features or components.

As used herein, the term “including” as well as other forms, such as “include”, “includes” and “included” is not limiting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” refers to a product obtained by reaction of the compound of the present invention with a suitable acid or a base. Pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention (compound of formula (I) or (II)) can form pharmaceutically acceptable salts with various organic bases such as lysine, arginine, guanidine, diethanolamine or metformin. Suitable base salts include, but are not limited to, aluminum, calcium, lithium, magnesium, potassium, sodium, or zinc salts.

As used herein, the term “stereoisomer” is a term used for all isomers of individual compounds of formula (I) or formula (II) that differ only in the orientation of their atoms in space. The term stereoisomer includes mirror image isomers (enantiomers) of compounds of formula (I) or formula (II), mixtures of mirror image isomers (racemates, racemic mixtures) of compounds of formula (I) or formula (II), geometric (cis/trans or E/Z, R/S) isomers of compounds of formula (I) or formula (II) and isomers of compounds of formula (I) or formula (II) with more than one chiral center that are not mirror images of one another (diastereoisomers).

The term “treatment”/“treating” means any treatment of a disease, disorder or condition in a mammal, including: (a) inhibiting the disease, i.e., slowing or arresting the development of clinical symptoms; and/or (b) relieving the disease, i.e., causing the regression of clinical symptoms and/or (c) alleviating or abrogating a disease and/or its attendant symptoms.

As used herein, the terms “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject,” that is interchangeable with ‘patient’, refers to an animal, preferably a mammal, and most preferably a human. Subjects include primates and other mammals such as equines, cattle, swine, sheep, poultry and pets in general.

As used herein, the term, “therapeutically effective amount” refers to an amount of a compound of formula (I) or formula (II) or a pharmaceutically acceptable salt or a stereoisomer thereof; or a composition comprising the compound of formula (I) or formula (II), or a pharmaceutically acceptable salt or a stereoisomer thereof, effective in producing the desired therapeutic response in a particular patient suffering from a disease or disorder mediated by kinase enzymes, particularly IRAK or IRAK-4 enzyme. Particularly, the term “therapeutically effective amount” includes the amount of the compound of formula (I) or formula (II) or a pharmaceutically acceptable salt or a stereoisomer thereof, which, when administered, induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment. The therapeutically effective amount of the compound or composition can be varied with the particular condition being treated, the severity of the condition being treated or prevented, the duration of the treatment, the nature of concurrent therapy, the age and physical condition of the subject, and the specific compound or composition employed the particular pharmaceutically acceptable carrier utilized.

In one certain embodiment, the present invention provides a pharmaceutical composition comprising a compound described herein, admixed with a pharmaceutically acceptable carrier or diluent.

As used herein, the term “composition” is intended to encompass a product comprising the specified ingredients, as well as any product which results, directly or indirectly, from combination of the specified ingredients.

As used herein, the term “pharmaceutical composition” refers to a composition(s) containing a therapeutically effective amount of at least one compound of formula (I) or (II) or a pharmaceutically acceptable salt thereof; and a pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories; or parenterally, for example intravenously, intramuscularly or subcutaneously; in the form of injectable sterile solutions or suspensions; topically, for example in the form of ointments or creams or transdermals, in the form of patches; or in other ways, for example in the form of aerosols or nasal sprays.

The pharmaceutical composition(s) usually contain(s) about 1% to about 99%, for example, about about 5% to about 75%, or from about 10% to about 30% by weight of the compound of formula (I) or (II) or pharmaceutically acceptable salts thereof. The amount of the compound of formula (I) or (II) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of about 1 mg to about 1000 mg or higher or lower than the afore mentioned range.

The present invention also provides methods for formulating the disclosed compounds as for pharmaceutical administration.

The compositions and methods of the present invention may be utilized to treat a subject in need thereof. In certain embodiments, the subject is a mammal such as a human, or a non-human mammal. When administered to an animal, such as a human, the composition or the compound is preferably administered as a pharmaceutical composition comprising, for example, a compound of formula (I) or (II) and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. The examples of carriers, stabilizers and adjuvants can be found in literature, Osol, A. and J. E. Hoover, et al. (eds.), Remington's Pharmaceutical Sciences, 15^{th} Ed., Easton, Mack Publ. Co., PA [1975].

In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as the compounds of the present invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable carrier” as used herein refers to a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious or hazardous to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using a binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as, for example, dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein mean the modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, and vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13^{th} ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The subject or patient receiving this treatment is any animal in need, including primates, preferably humans, and other mammals such as equines, cattle, swine and sheep; and poultry and pets in general.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The compounds of the present invention may be administered in combination with one or more other drugs (1) to complement and/or enhance prevention and/or therapeutic efficacy of the preventive and/or therapeutic drug effect of the compound of the present invention, (2) to modulate pharmacodynamics, improve absorption improvement, or reduce dosage reduction of the preventive and/or therapeutic compound of the present invention, and/or (3) to reduce or ameliorate the side effects of the preventive and/or therapeutic compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The respective compounds may be administered by the same or different route and the same or different method.

A concomitant medicine comprising the compounds of the present invention and other drug may be administered as a combination preparation in which both components are contained in a single formulation, or administered as separate formulations. The administration by separate formulations includes simultaneous administration and or administration of the formulations separated by some time intervals. In the case of the administration with some time intervals, the compound of the present invention can be administered first, followed by another drug or another drug can be administered first, followed by the compound of the present invention, so long as the two compounds are simultaneously active in the patient at least some of the time during the conjoint therapy. The administration method of the respective drugs may be administered by the same or different route and the same or different method.

The dosage of the other drug can be properly selected, based on a dosage that has been clinically used, or may be a reduced dosage that is effective when administered in combination with a compound of the present invention. The compounding ratio of the compound of the present invention and the other drug can be properly selected according to age and weight of a subject to be administered, administration method, administration time, disorder to be treated, symptom and combination thereof. For example, the other drug may be used in an amount of about 0.01 to about 100 parts by mass, based on 1 part by mass of the compound of the present invention. The other drug may be a combination of two or more drugs in a proper proportion. The other drug that complements and/or enhances the preventive and/or therapeutic efficacy of the compound of the present invention includes not only those that have already been discovered, but those that may be discovered in future.

Diseases on which this concomitant use exerts a preventive and/or therapeutic effect are not particularly limited. The concomitant medicine can be used to treat any diseases discussed herein, as long as it complements and/or enhances the preventive and/or therapeutic efficacy of the compound of the present invention.

For example, in the methods of the invention directed to the treatment of cancer, the compound of the present invention can be used with an existing chemotherapeutic conjointly using a single pharmaceutical composition or a combination of different pharmaceutical compositions concomitantly or in a mixture form. Examples of the chemotherapeutic include an alkylation agent, nitrosourea agent, antimetabolite, anticancer antibiotics, vegetable-origin alkaloid, topoisomerase inhibitor, hormone drug, hormone antagonist, aromatase inhibitor, P-glycoprotein inhibitor, platinum complex derivative, other immunotherapeutic drugs and other anticancer drugs. Further, it a compound of the invention can be used administered conjointly with a cancer treatment adjunct, such as a leucopenia (neutropenia) treatment drug, thrombocytopenia treatment drug, antiemetic and cancer pain intervention drug, concomitantly or in a mixture form. Chemotherapeutic agents that may be conjointly administered with compounds of the invention include: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, and vinorelbine.

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, with cryotherapy, or with any combination of these.

In certain embodiments, different compounds of the invention may be conjointly administered with one or more other compounds of the invention. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound of the invention provides a synergistic effect. In certain embodiments, conjointly administering one or more additional chemotherapeutics agents provides an additive effect.

The drugs for conjoint therapy include, for example, antibacterial agents, antifungal agents, antibiotics, sedatives, anesthetics, antidepressants, antiulcer drugs, antiarrhythmic agents, antiprotozoal agents, hypotensive diuretic drugs, anticoagulants, tranquilizers, antipsychotics, antitumor drugs, hypolipidemic drugs, muscle relaxants, antiepileptic drugs, antitussives and expectorant drugs, antiallergic drugs, cardiac stimulants, hypotensive diuretics, therapeutic drugs for arrhythmia, vasodilators, vasoconstrictors, therapeutic drugs for diabetes, antinarcotics, vitamins, vitamin derivatives, antiasthmatics, therapeutic agents for atopic dermatitis, therapeutic agents for pollakisuria/anischuria, antipruritic drugs, therapeutic agents for allergic rhinitis, hypertensors, endotoxin-antagonists or -antibodies, signal transduction inhibitors, inhibitors of anti-inflammatory mediator activity, inhibitors of inflammatory mediator activity, antibodies to inhibit inflammatory mediator activity, antibodies to inhibit anti-inflammatory mediator activity and the like.

In certain embodiments, the present invention relates to a compound or a pharmaceutically acceptable salt or a stereoisomer thereof, for use as a medicament.

In further embodiments, the present invention relates to a method of treating an IRAK-4 mediated disorder or disease or condition in a subject comprising administering a therapeutically effective amount of a compound of formula (I) or (II), or pharmaceutically acceptable salts thereof.

In certain embodiments, the present invention relates to a method of treating disorders or diseases or condition mediated by MyD88 in a subject comprising administering a therapeutically effective amount of a compound of formula (I) or (II) or pharmaceutically acceptable salts thereof.

In certain embodiments, the IRAK-4-mediated disorder or disease or condition is selected from a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder and a CNS disorder.

In certain embodiments, the IRAK-4-mediated disorder or disease or condition is selected from a cancer, an inflammatory disorder, an autoimmune disease, metabolic disorder, a hereditary disorder, a hormone-related disease, immunodeficiency disorders, a condition associated with cell death, a destructive bone disorder, thrombin-induced platelet aggregation, liver disease, pathologic immune conditions involving T cell activation and a cardiovascular disorder.

In any one of the foregoing embodiments, the cancer or proliferative disorder is selected from a solid tumor, benign or malignant tumor, carcinoma of the brain, kidney, liver, stomach, vagina, ovaries, gastric tumors, breast, bladder colon, prostate, pancreas, lung, cervix, testis, skin, bone or thyroid; sarcoma, glioblastomas, neuroblastomas, multiple myeloma, gastrointestinal cancer, a tumor of the neck and head, an epidermal hyperproliferation, psoriasis, prostate hyperplasia, a neoplasia, adenoma, adenocarcinoma, keratoacanthoma, epidermoid carcinoma, large cell carcinoma, non-small-cell lung carcinoma, Hodgkins and Non-Hodgkins lymphomas, a mammary carcinoma, follicular carcinoma, papillary carcinoma, seminoma, melanoma; hematological malignancies selected from leukemia, diffuse large B-cell lymphoma (DLBCL), activated B-cell-like DLBCL, chronic lymphocytic leukemia (CLL), chronic lymphocytic lymphoma, primary effusion lymphoma, Burkitt lymphoma/leukemia, acute lymphocytic leukemia, B-cell pro lymphocytic leukemia, acute myeloid leukemia (AML), chronic myeloid leukemia (CML), lymphoplasmacytic lymphoma, Waldenstrom's macroglobulnemia (WM), splenic marginal zone lymphoma, intravascular large B-cell lymphoma, plasmacytoma and multiple myeloma.

In any of the forgoing embodiments, the neurodegenerative disease may be selected from Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy and graft versus host disease.

In any one of the forgoing embodiments, the inflammatory disorder may be selected from ocular allergy, conjunctivitis, keratoconjunctivitis sicca, vernal conjunctivitis, allergic rhinitis, autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, primary biliary cirrhosis, uveitis (anterior and posterior), Sjogren's syndrome, interstitial lung fibrosis, psoriatic arthritis, systemic juvenile idiopathic arthritis, nephritis, vasculitis, diverticulitis, interstitial cystitis, glomerulonephritis (e.g., including idiopathic nephrotic syndrome or minimal change nephropathy), chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS) and osteoarthritis.

In preferred embodiments, the present invention relates to a method of treating disorders or diseases or condition mediated by L265P somatic mutation of MyD88 in a subject comprising administering a therapeutically effective amount of a compound of formula (I), (IA), (IB), (IC), (ID), (II), (IIA), (III) or (IV).

Such disorders, diseases, or conditions associated with an MYD88 mutation include cancers, inflammatory disorders such as ulcerative colitis, autoimmune diseases, metabolic disorders, hereditary disorders, hormone-related diseases, immunodeficiency disorders, conditions associated with cell death, destructive bone disorders, thrombin-induced platelet aggregation, liver disease and cardiovascular disorder.

In any of the foregoing embodiments, the diseases mediated by L265P somatic mutation of MyD88 are hematological tumors such as lymphoma. In preferred embodiments, the diseases mediated by a L265P somatic mutation of MyD88 are Waldenstrom's macroglobulnemia or diffuse large B-cell lymphoma.

In certain embodiments, the present invention provides compounds of formula (I), (IA), (IB), (IC), (ID), (II), (IIA), (III) or (IV), or a pharmaceutically acceptable salt or a stereoisomer thereof, for use for the treatment of a cancer, an inflammatory disorder, an autoimmune disease, a metabolic disorder, a hereditary disorder, a hormone-related disease, immunodeficiency disorders, a condition associated with cell death, a destructive bone disorder, thrombin-induced platelet aggregation, liver disease, pathologic immune conditions involving T cell activation and a cardiovascular disorder.

In certain embodiments, the present invention provides a use of the compounds of formula (I), (IA), (IB), (IC), (ID), (II), (IIA), (III) or (IV), or a pharmaceutically acceptable salt or a stereoisomer thereof, in the manufacture of a medicament for the treatment of cancer, an inflammatory disorder, an autoimmune disease, a metabolic disorder, a hereditary disorder, a hormone-related disease, immunodeficiency disorders, a condition associated with cell death, a destructive bone disorder, thrombin-induced platelet aggregation, liver disease and a cardiovascular disorder.

Some embodiments provide a method of inhibiting IRAK-4-mediated signaling in a cell expressing IRAK-4, comprising contacting the cell with at least one compound as disclosed herein, or a pharmaceutically acceptable salt or a stereoisomer thereof.

The IRAK-4 inhibitor compounds according to of formula (I) or formula (II) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures. Moreover, by utilizing the procedures described in detail, one of ordinary skill in the art can prepare additional compounds of the present invention claimed herein. All temperatures are in degrees Celsius (° C.) unless otherwise noted.

In certain embodiments, the compounds of the present invention can also contain unnatural proportions of atomic isotopes at one or more of the atoms that constitute such compounds. For example, the present invention also embraces isotopically-labeled variants of compounds of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present invention can generally be prepared by following procedures analogous to those disclosed in the Schemes and/or in the Examples herein below, by substituting an isotopically labeled reagent for a non-isotopically labeled reagent.

The MS (Mass Spectral) data provided in the examples were obtained using the following equipment: API 2000 LC/MS/MS/Triplequad; Agilent (1100) Technologies/LC/MS/DVL/Singlequad and Shimadzu LCMS-2020/Singlequad.

The NMR data provided in the examples were obtained using the equipment—¹HNMR: Varian—300, 400 and 600 MHz.

The abbreviations used in the entire specification may be summarized herein below with their particular meaning.

° C. (degree Celsius); δ (delta); % (percentage); Ac₂O (Acetic anhydride); (BOC)₂O (Boc anhydride); bs (Broad singlet); CDCl₃ (Deuterated chloroform); CH₂Cl₂/DCM (Dichloromethane); DAST (Diethylaminosulfur trifluoride); DMF (Dimethyl formamide); DMSO (Dimethyl sulphoxide); DIPEA/DIEA (N,N-Diisopropyl ethylamine); DMAP (Dimethyl aminopyridine); (DMSO-d₆ (Deuterated DMSO); d (Doublet); dd (Doublet of doublet); EDCI.HCl (1-(3-Dimethyl aminopropyl)-3-carbodiimide hydrochloride); EtOAc (Ethyl acetate); EtOH (Ethanol); Fe (Iron powder); g or gm (gram); HATU (1-[bis(dimethylamino)methylene]-1H-1,2,3-triazolo[4,5-b]pyridinium 3-oxid hexafluorophosphate); H or H₂ (Hydrogen); H₂O (Water); HOBt (1-Hydroxy benzotriazole); H₂SO₄ (Sulphuric acid); HCl (Hydrochloric acid); h or hr (Hours); Hz (Hertz); HPLC (High-performance liquid chromatography); J (Coupling constant); K₂CO₃ (Potassium carbonate); KOAc (Potassium Acetate); KNO₃ (Potassium nitrate); LiOH (Lithium hydroxide); MeOH/CH3OH (Methanol); mmol (Millimol); M (Molar); ml (Millilitre); mg (Milligram); m (Multiplet); mm (Millimeter); MHz (Megahertz); min (Minutes); NaH (Sodium hydride); NaHCO₃ (Sodium bicarbonate); Na₂SO₄ (Sodium sulphate); N₂ (Nitrogen); NMR (Nuclear magnetic resonance spectroscopy); Pd/C (palladium carbon); Pd(dppf)Cl₂ (1,1′-Bis(diphenylphosphino)ferrocene) palladium(II)dichloride; RT (Room Temperature); S (Singlet); TBDMS (Tertiary butyldimethylsilyl chloride); TEA (Triethylamine); TFA (Trifluoroaceticacid); TLC (Thin Layer Chromatography); THF (Tetrahydrofuran); t (Triplet); Zn(CN)₂ (Zinc Cyanide).

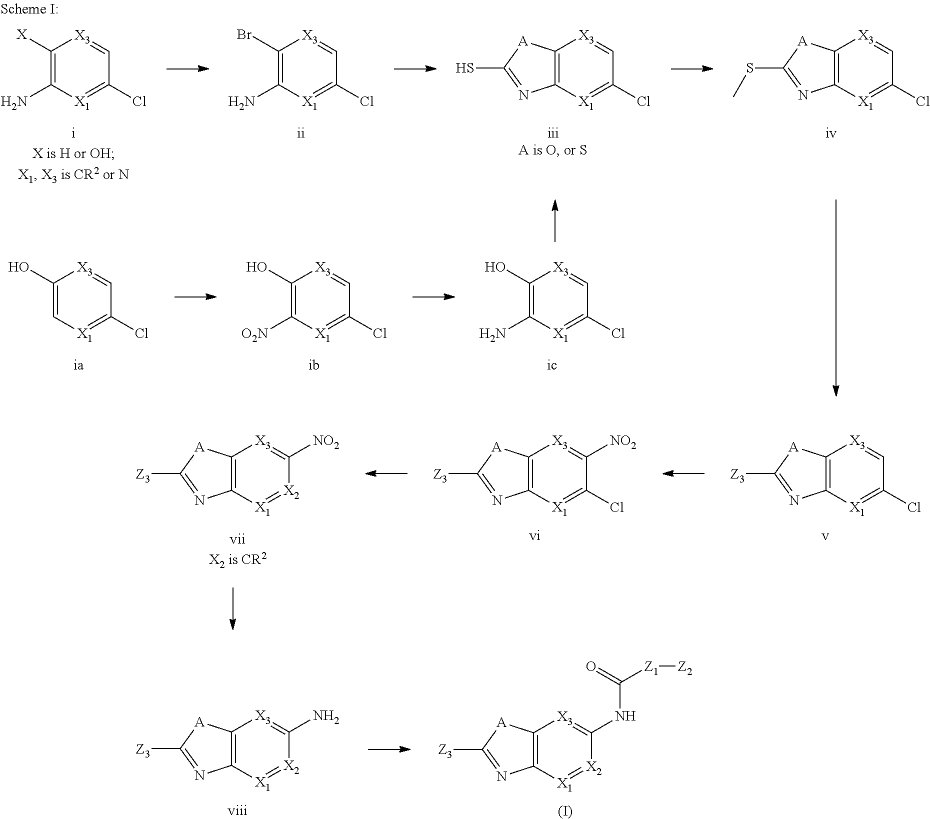

The first general approach for the synthesis of compound of formula (I) is depicted in general scheme I. Compound of formula ii was obtained from compound of formula i by reacting with bromine at certain temperature. Compound of formula ii was cyclized by using potassium ethyl xanthate to give compound of formula iii.

Compound of formula iii was obtained also by different method as follows. Compound of formula ib was obtained from compound of formula ia by nitrating with potassium nitrate at certain temperature. Compound ib was reduced with zinc and ammonium chloride gave compound of formula ic. Compound of formula ic which was cyclized using potassium ethyl xanthate to give compound of formula iii.

Compound of formula iii on alkylation with alkyl halides by using bases like potassium carbonate gave compound of formula iv, which on further substitution by appropriate amines gave compound of formula v. Compound of formula vi was obtained from compound of formula v by nitrating with potassium nitrate at certain temperature. Compound vi was treated with amines at certain temperature gave compound of formula vii. Reduction of compound of formula vii with suitable reducing reagents like Zn and ammonium chloride gave compound of formula viii. Compound of formula viii was treated with conventional amide coupling with a suitable acid of compound of formula vi by using standard amide coupling reagent known in the literature to give compound of formula (I).

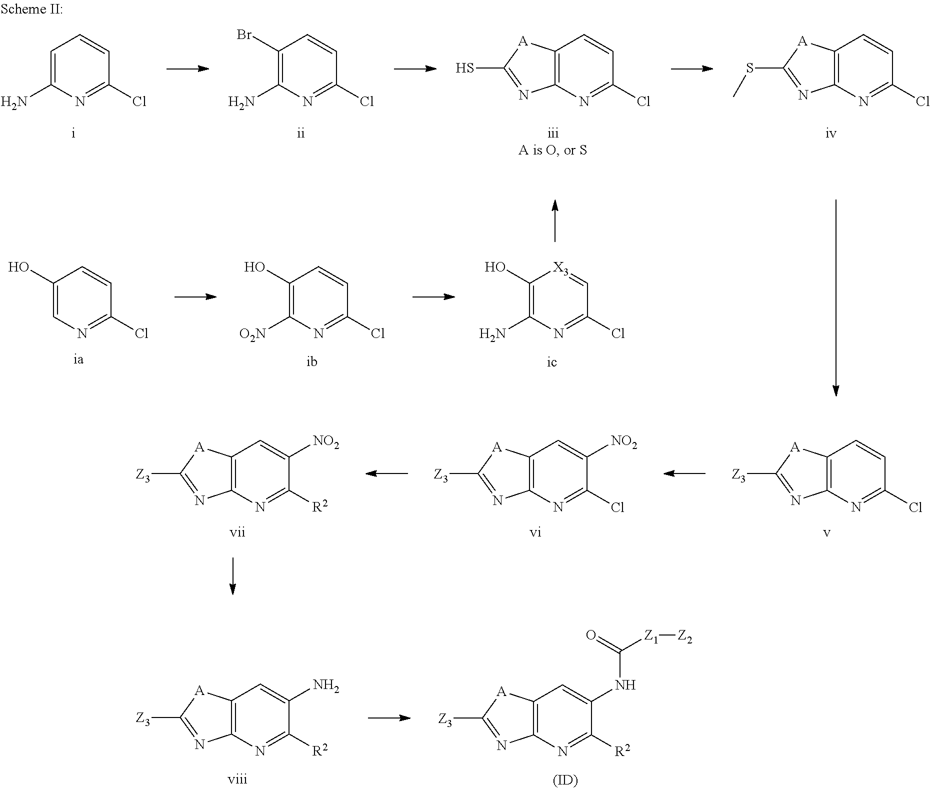

The first general approach for the synthesis of compound of formula (ix) is depicted in general scheme II. Compound of formula ii was obtained from compound of formula i by reacting with bromine at certain temperature. Compound of formula ii which underwent cyclization with potassium ethyl xanthate gave compound of formula iii.

Compound of formula ib was obtained from compound of formula ia by nitrating with potassium nitrate at certain temperature. Compound ib was reduced with zinc and ammonium chloride gave compound of formula ic. Compound of formula ic which underwent cyclization with potassium ethyl xanthate gave compound of formula iii.

Compound of formula iii on alkylation with alkyl halides using base like potassium carbonate gave compound of formula iv, which on further displacement with amines gave compound of formula v. Compound of formula vi was obtained from compound of formula v by nitrating with potassium nitrate at certain temperature. Compound vi was treated with amines at certain temperature gave compound of formula vii. Reduction of compound of formula vii with suitable reducing reagents like Zn and ammonium chloride gave compound of formula viii. Compound of formula viii was treated with Amide coupling with a suitable acid of compound of formula vi by using standard amide coupling reagent known in the literature to give compound of formula (ID).

The below compounds were prepared by procedure similar to the one described in WO2011/043371, WO2013/59587, WO2013/106535 and WO2012097013 with appropriate variations in reactants, quantities of reagents at suitable reaction conditions. The characteristics of the compounds are summarized herein below table.

| Intermediate | Structure | Analytical data |
|---|---|---|
| 1 | 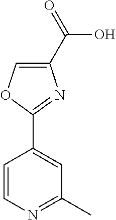 | ¹HNMR (300 MHz, DMSO-d₆): δ 13.2 (bs, 1H), 8.97 (s, 1H), 8.66-8.64 (d, 1H), 7.82 (s, 1H), 7.73- 7.72 (d, 1H), 2.57 (s, 3H). LCMS: m/z = 205.2 (M + 1)⁺; HPLC: 98.93%. |
| 2 | 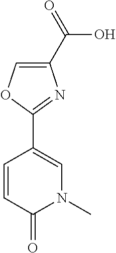 [SMILES: Cn1cc(-c2nc(C(=O)O)co2)ccc1=O] | ¹HNMR (300 MHz, DMSO-d₆): δ 8.77 (s, 1H), 8.54 (s, 1H), 7.91 (dd, 1H), 6.50 (d, 1H), 3.58 (s, 3H). |
| 3 | 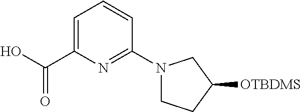 [SMILES: CC(C)(C)[Si](C)(C)O[C@H]1CCN(c2cccc(C(=O)O)n2)C1] | ¹HNMR (300 MHz, DMSO-d₆): δ 7.63-7.58 (t, 1H), 7.23-7.20 (d, 1H), 6.66-6.63 (d, 1H), 4.56 (bs, 1H), 3.6-3.2 (m, 2H), 2.10-2.05 (m, 1H), 1.88 (bs, 1H), 1.38 (s, 2H), 0.85 (s, 9H), 0.08 (s, 6H). LCMS: m/z = 323.3 (M + 1)⁺. |
| 4 | 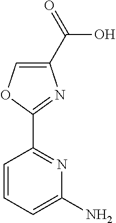 [SMILES: Nc1cccc(-c2nc(C(=O)O)co2)n1] | ¹HNMR (300 MHz, DMSO-d₆): δ 13.2 (bs, 1H), 8.81 (s, 1H), 7.54 (t, 1H), 7.26 (d, 1H), 6.57 (d, 1H), 6.34 (bs, 2H), LCMS: m/z = 205.6 (M + 1)⁺. |
| 5 | 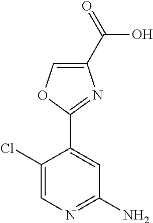 [SMILES: Nc1cc(-c2nc(C(=O)O)co2)c(Cl)cn1] | ¹HNMR (300 MHz, DMSO-d₆): δ 8.18 (s, 1H), 8.03 (s, 1H), 7.05 (s, 1H), 6.4 (bs, 2H), LCMS: m/z = 239.7 (M + 1)⁺. |
| 6 | 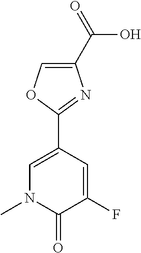 [SMILES: Cn1cc(-c2nc(C(=O)O)co2)cc(F)c1=O] | ¹HNMR (300 MHz, DMSO-d₆): δ 13.2 (bs, 1H), 8.81 (s, 1H), 8.46-8.45 (t, 1H), 7.85-7.81 (dd, 1H), 3.59 (s, 3H). LCMS: m/z = 239.1 (M + 1)⁺; HPLC: 97.25%. |
| 7 | 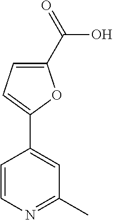 | ¹HNMR (300 MHz, DMSO-d₆): δ 8.54-8.52 (d, 1H) 7.64 (s, 1H), 7.57-7.56 (d, 1H), 7.42-7.41 (d, 1H), 7.38-7.37 (d, 1H), 2.53 (s, 3H). LCMS: m/z = 203.9 (M + 1)⁺. |
| 8 | 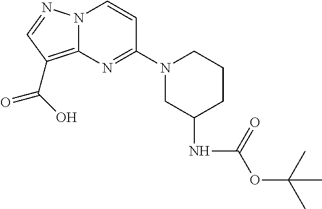 [SMILES: CC(C)(C)OC(=O)NC1CCCN(c2ccn3ncc(C(=O)O)c3n2)C1] | ¹HNMR (300 MHz, DMSO-d₆): δ 11.6 (bs, 1H), 8.6 (s, 1H), 8.2 (s, 1H), 6.99 (d, 1H), 6.70 (d, 1H), 4.22-4.1 (m, 2H), 3.22-3.0 (m, 2H), 1.82-1.6 (m, 2H), 1.50-1.30 (m, 11H). LCMS: m/z = 362.0 (M + 1)⁺. |
| 9 | 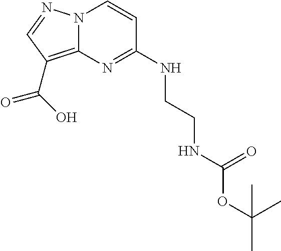 [SMILES: CC(C)(C)OC(=O)NCCNc1ccn2ncc(C(=O)O)c2n1] | LCMS: m/z = 322.1 (M + 1)⁺. |
| 10 | 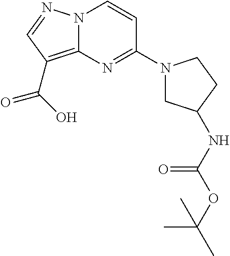 [SMILES: CC(C)(C)OC(=O)NC1CCN(c2ccn3ncc(C(=O)O)c3n2)C1] | ¹HNMR (300 MHz, DMSO-d₆): δ 11.7 (bs, 1H), 8.64 (s, 1H), 8.10 (s, 1H), 7.30-7.25 (m, 1H), 6.46 (d, 1H), 4.22-4.0 (m, 1H), 3.8-3.6 (m, 2H), 3.5-3.4 (m, 2H), 2.22-1.8 (m, 2H), 1.35 (s, 9H). LCMS: m/z = 348.0 (M + 1)⁺. |
| 11 | 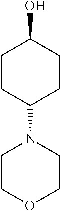 | LCMS: m/z = 186.2 (M + 1)⁺. |

### EXAMPLE 1

### N-(2-(4-methylpiperazin-1-yl)-5-(piperidin-1-yl)thiazolo[4,5-b]pyridin-6-yl)-2-(2-methylpyridin-4-yl)oxazole-4-carboxamide hydrochloride

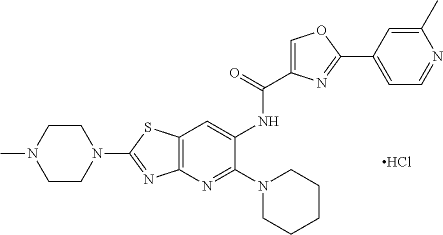

### Step 1: Preparation of 3-bromo-6-chloropyridin-2-amine

To a solution of 2-amino-6-chloropyridine (15 g, 116 mmol) in chloroform (600 ml) was added a solution of bromine (4.2 g, 965 mmol) in chloroform (50 ml) at 0° C. and the reaction mixture was stirred at room temperature for 16 h. After completion of reaction, the reaction was quenched over ice cold water; extracted to DCM and concentrated to obtain the crude compound. The crude compound was purified by silica gel column chromatography using 10% ethyl acetate in hexane as eluent to afford the title compound (6.2 g, 25.5%). LCMS: m/z=209.0 (M+1)⁺.

### Step 2: Preparation of 5-chlorothiazolo[4,5-b]pyridine-2-thiol

A solution of 3-bromo-6-chloropyridin-2-amine (42 g, 202 mmol) and potassium ethyl xanthate (58.15 g, 363 mmol) in DMF (200 mL) was heated at 150° C. for 4 h. The reaction mixture was cooled to 0° C., diluted with ice water, acidified with conc. HCl. The solid obtained was filtered and dried under vacuum to afford the title compound (40 gm, 69%). LCMS: m/z=203.0 (M+1)⁺

### Step 3: Preparation of 5-chloro-2-(methylthio)thiazolo[4,5-b]pyridine

To a stirred solution of 5-chlorothiazolo[4,5-b]pyridine-2-thiol (37 g, 181.3 mmol) in ethyl acetate (200 mL) was added potassium carbonate (50 g, 362 mmol) and methyl iodide (38.9 g, 272 mmol). Then the reaction mixture was stirred at RT for 2 h. After completion of the reaction, the reaction mixture was diluted with water; extracted with ethyl acetate, dried over sodium sulphate and concentrated to afford the title compound (27 g, 70%). LCMS: m/z=217.6 (M+1)⁺.

### Step 4: Preparation of 5-chloro-2-(4-methylpiperazin-1-yl)thiazolo[4,5-b]pyridine

To a solution of 5-chloro-2-(methylthio)thiazolo[4,5-b]pyridine (750 mg, 3.47 mmol) in THF (5 mL) was added N-methyl piperazine (3 mL) and the reaction mixture was heated at 75° C. overnight. After completion of reaction, the mixture was evaporated under reduced pressure. The residue was diluted with water and filtered and the solid was suction dried to afford the title compound (735 mg, 79%). LCMS: m/z=271.1 (M+2)⁺.

### Step 5: Preparation of 5-chloro-2-(4-methylpiperazin-1-yl)-6-nitrothiazolo[4,5-b]pyridine

Potassium nitrate (447 mg) was added portion wise to a mixture of 5-chloro-2-(4-methylpiperazin-1-yl)thiazolo[4,5-b]pyridine (600 mg, 2.23 mmol) in concentrated sulphuric acid (6 ml) at 0° C. The reaction mixture was stirred at room temperature for 16 h. After the completion of the reaction, which was poured in crushed ice and the solid formed was filtered and dried to get the title compound (505 mg, 72.4%). LCMS: m/z=314.10 (M+1)⁺.

### Step 6: Preparation of 2-(4-methylpiperazin-1-yl)-6-nitro-5-(piperidin-1-yl)thiazolo[4,5-b]pyridine

A solution of piperidine (2 mL) and 5-chloro-2-(4-methylpiperazin-1-yl)-6-nitrothiazolo[4,5-b]pyridine (200 mg, 0.678 mmol) was stirred at 70° C. for 2 h. The reaction mixture was concentrated and diluted with water. The solid formed was filtered and suction dried to get crude product which was then purified by silica gel column chromatography using DCM as eluent to get the title compound (175 mg, 71%). LCMS: m/z=363.0 (M+1)⁺.

### Step 7: Preparation of 2-(4-methylpiperazin-1-yl)-5-(piperidin-1-yl)thiazolo[4,5-b]pyridin-6-amine

To a solution of 2-(4-methylpiperazin-1-yl)-6-nitro-5-(piperidin-1-yl)thiazolo[4,5-b]pyridine (174 Mg, 0.479 mmol) in THF (20 mL) was added ammonium chloride (207 mg, 3.83 mmol) in water (4 mL) and zinc dust (249 mg, 3.83 mmol). Then the reaction mixture was stirred at room temperature for 1 h. The catalyst was filtered through Celite® and the filtrate was extracted with ethyl acetate. The organic solvent was distilled out to get the title compound (151 mg, 94.9%). LCMS: m/z=333.1 (M+1)⁺.

### Step 8: Preparation of N-(2-(4-methylpiperazin-1-yl)-5-(piperidin-1-yl)thiazolo[4,5-b]pyridin-6-yl)-2-(2-methylpyridin-4-yl)oxazole-4-carboxamide hydrochloride

A solution of 2-(4-methylpiperazin-1-yl)-5-(piperidin-1-yl)thiazolo[4,5-b]pyridin-6-amine (150 mg, 0.45 mmol), intermediate 1 (92 mg, 0.45 mmol), HATU (256 mg, 0.675 mmol) and DIPEA (232 mg, 1.801 mmol) in DMF (10 mL) was stirred at RT overnight. Then the reaction mixture was quenched with ice water; extracted with ethyl acetate; dried over sodium sulphate and concentrated to obtain the crude compound. The residual solid was triturated with diethyl ether, filtered and dried over vacuum to afford the title compound. This was treated with methanol/methanolic HCL (5/5 mL) to afford the title compound as hydrochloride salt (111 mg).

¹HNMR (400 MHz, CDCl₃): δ 9.89 (s, 1H), 9.03 (s, 1H), 8.70-8.69 (d, 1H), 8.39 (s, 1H), 7.83 (s, 1H), 7.74-7.72 (d, 1H), 3.75-3.70 (t, 4H), 3.13-3.11 (t, 4H), 2.67 (s, 3H), 2.54-2.52 (t, 4H), 2.35 (s, 3H), 1.93-1.88 (m, 4H), 1.75-1.65 (m, 2H). HPLC: 94.50%; LCMS: m/z=519.1 (M+1)⁺.

The below compounds were prepared by procedure similar to the one described in example 1 with appropriate variations in reactants, quantities of reagents at suitable reaction conditions. The physicochemical characteristics of the compounds are summarized herein below table. Example 6 was prepared by procedure similar to the one described in WO2013/106535.

| Example | Structure | Analytical data |
|---|---|---|
| 2 | 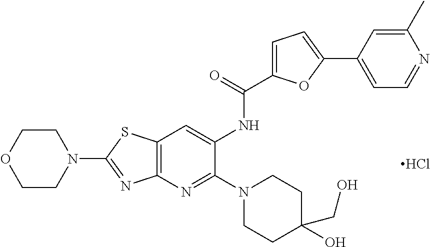 [SMILES: Cc1cc(-c2ccc(C(=O)Nc3cc4sc(N5CCOCC5)nc4nc3N3CCC(O)(CO)CC3)o2)ccn1 ; Cl] | ¹HNMR (400 MHz, CD₃OD): δ 8.71 (d, 1H), 8.47 (d, 2H), 8.35 (d, 1H), 7.78 (d, 1H), 7.57 (d, 1H), 3.90-3.70 (m, 10H), 3.70-3.50 (m, 4H), 3.39 (s, 2H), 2.83 (s, 3H), 2.10-1.90 (m, 2H), 1.80-1.60 (m, 2H); LCMS: m/z = 551.30 (M + 1)⁺; HPLC: 95.78% |
| 3 | 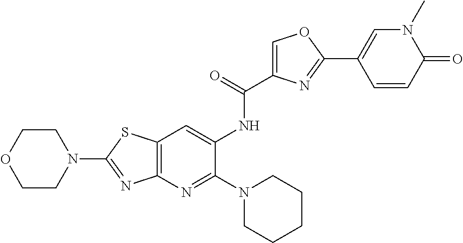 [SMILES: Cn1cc(-c2nc(C(=O)Nc3cc4sc(N5CCOCC5)nc4nc3N3CCCCC3)co2)ccc1=O] | ¹HNMR (300 MHz, CDCl₃): δ 9.75 (s, 1H), 9.04 (s, 1H), 8.26 (s, 1H), 8.16 (d, 1H), 7.97-7.93 (m, 1H), 6.71 (d, 1H), 3.84- 3.81 (m, 4H), 3.70-3.66 (m, 7H), 3.13-3.09 (m, 4H), 1.90-1.75 (m, 4H), 1.80-1.60 (m, 2H). LCMS: m/z = 522.30 (M + 1)⁺; HPLC: 96.27% |
| 4 | 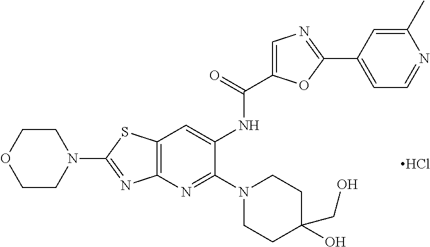 [SMILES: Cc1cc(-c2ncc(C(=O)Nc3cc4sc(N5CCOCC5)nc4nc3N3CCC(O)(CO)CC3)o2)ccn1 ; Cl] | ¹HNMR (400 MHz, DMSO-d₆): δ 9.72 (s, 1H), 9.18 (s, 1H), 9.00 (s, 1H), 8.78 (d, 1H), 8.10 (bs, 2H), 3.74-3.58 (m, 5H), 3.33 (s, 3H), 3.20-3.14 (m, 3H), 3.03-3.00 (m, 3H), 2.71-2.66 (m, 3H), 2.13-2.10 (m, 2H), 1.56-1.53 (m, 4H). LCMS: m/z = 552.30 (M + 1)⁺; HPLC: 97.92% |
| 5 | 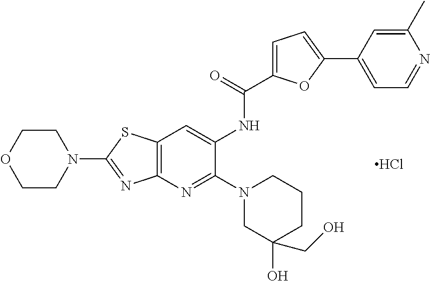 [SMILES: Cc1cc(-c2ccc(C(=O)Nc3cc4sc(N5CCOCC5)nc4nc3N3CCCC(O)(CO)C3)o2)ccn1 ; Cl] | ¹HNMR (400 MHz, DMSO-d₆): δ 10.0 (bs, 1H), 8.85 (d, 1H), 8.63 (s, 1H), 8.47 (s, 1H), 8.36 (d, 1H), 7.92 (d, 1H), 7.60 (d, 1H), 3.90-3.50 (m, 8H), 3.45-2.90 (m, 7H), 2.85-2.75 (m, 4H), 2.05-1.85 (m, 1H), 1.65-1.40 (m, 3H); LCMS: m/z = 551.30 (M + 1)⁺; HPLC: 98.38% |
| 6 | 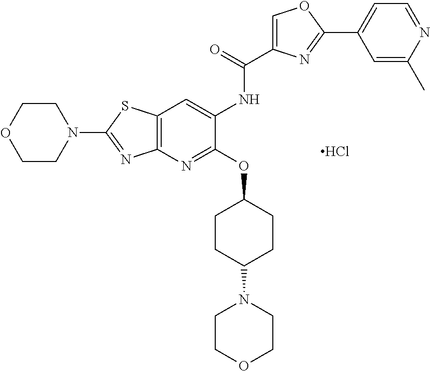 | ¹HNMR (400 MHz, DMSO-d₆): δ 9.40 (s, 1H), 9.06 (s, 1H), 8.85 (s, 1H), 8.71 (d, 1H), 7.81 (s, 1H), 7.74 (d, 1H), 5.10-5.06 (m, 2H), 3.74-3.72 (m, 4H), 3.38-3.28 (m, 4H), 3.59-3.57 (m, 8H), 2.66-2.50 (m, 3H), 2.32-2.18 (m, 2H), 1.94-1.90 (m, 2H), 1.56-1.50 (m, 4H), LCMS: m/z = 606.30 (M + 1)⁺; HPLC: 98.78% |
| 7 | 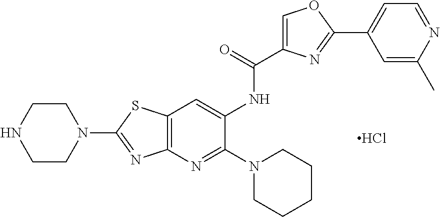 [SMILES: Cc1cc(-c2nc(C(=O)Nc3cc4sc(N5CCNCC5)nc4nc3N3CCCCC3)co2)ccn1 ; Cl] | ¹HNMR (400 MHz, DMSO-d₆): δ 9.73 (s, 1H), 9.39-9.32 (m, 2H), 9.20 (s,1H), 8.96 (s, 1H), 8.86 (d, 1H), 8.12 (s, 1H), 8.05 (d, 1H), 3.90-3.83 (m, 4H), 3.35-3.26 (m, 4H), 3.19-3.01 (m, 4H), 2.71 (s, 3H), 1.90-1.82 (m, 4H), 1.78-1.64 (m, 2H). LCMS: m/z = 505.25 (M + 1)⁺; HPLC: 96.92% |
| 8 | 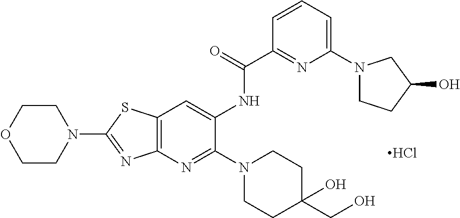 [SMILES: O=C(Nc1cc2sc(N3CCOCC3)nc2nc1N1CCC(O)(CO)CC1)c1cccc(N2CC[C@H](O)C2)n1 ; Cl] | ¹HNMR (400 MHz, DMSO-d₆): δ 10.38 (s, 1H), 9.04 (s, 1H), 7.71 (t, 1H), 7.36 (d, 1H), 6.73 (d, 1H), 4.43 (bs, 1H), 3.74-3.73 (m, 4H), 3.73-3.59 (m, 6H), 3.49-3.39 (m, 1H), 3.26-3.13 (m, 4H), 2.99-2.96 (m, 2H), 2.60-2.40 (m, 4H), 2.12-2.05 (m, 1H), 1.99-1.90 (m, 1H), 1.80-1.78 (m, 2H), 1.56-1.51 (m, 2H), LCMS: m/z = 556.00 (M + 1)⁺; HPLC: 97.46% |
| 9 | 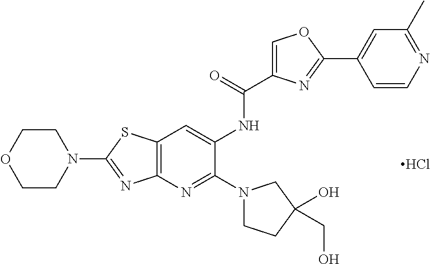 [SMILES: Cc1cc(-c2nc(C(=O)Nc3cc4sc(N5CCOCC5)nc4nc3N3CCC(O)(CO)C3)co2)ccn1 ; Cl] | ¹HNMR (300 MHz, CD₃OD): δ 8.94 (s, 1H), 8.91-8.89 (d, 1H), 8.59 (bs, 1H), 8.51-8.49 (d, 1H), 8.81 (s, 1H), 5.05 (m, 1H), 4.95 (m, 1H), 4.05 (m, 2H), 3.85-3.78 (m, 8H), 3.57 (m, 4H), 2.90 (s, 3H), 2.20 (m, 1H), 1.95 (m, 1H). LCMS: m/z = 538.3 (M + 1)⁺; HPLC: 96.13% |
| 10 | 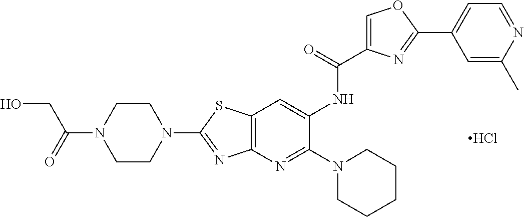 [SMILES: Cc1cc(-c2nc(C(=O)Nc3cc4sc(N5CCN(C(=O)CO)CC5)nc4nc3N3CCCCC3)co2)ccn1 ; Cl] | ¹HNMR (400 MHz, DMSO-d₆): δ 9.80 (s, 1H), 9.20 (s, 1H), 8.95 (s, 1H), 8.80-8.78 (d, 1H), 7.97 (s, 1H), 7.87-7.86 (d, 1H), 4.16 (s, 2H), 3.02 (t, 6H), 2.67 (s, 3H), 2.60-2.50 (m, 7H), 1.84-1.65 (m, 6H). LCMS: m/z = 563.4 (M + 1)⁺; HPLC: 95.15 % |
| 11 | 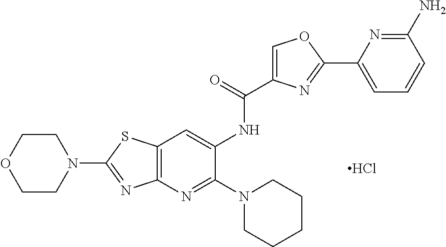 [SMILES: Nc1cccc(-c2nc(C(=O)Nc3cc4sc(N5CCOCC5)nc4nc3N3CCCCC3)co2)n1 ; Cl] | ¹HNMR (400 MHz, CD₃OD): δ 8.90 (s, 1H), 8.58 (bs, 1H), 8.09-8.05 (m, 1H), 7.63 (d, 1H), 7.21 (d, 1H), 3.86-3.76 (m, 8H), 3.55-3.40 (m, 4H), 1.84-1.71 (m, 6H). LCMS: m/z = 507.2 (M + 1)⁺; HPLC: 97.91% |
| 12 | 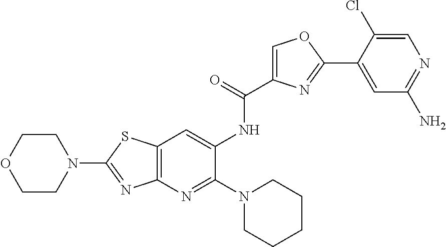 [SMILES: Nc1cc(-c2nc(C(=O)Nc3cc4sc(N5CCOCC5)nc4nc3N3CCCCC3)co2)c(Cl)cn1] | ¹HNMR (300 MHz, CDCl₃): δ 9.84 (s, 1H), 9.06 (s, 1H), 8.42 (s, 1H), 8.26 (s, 1H), 7.16 (s, 1H), 4.63 (s, 2H), 3.85-3.68 (m, 8H), 3.09 (t, 4H), 1.88-1.63 (m, 6H). LCMS: m/z = 541.0 (M + 1)⁺; HPLC: 97.38% |
| 13 | 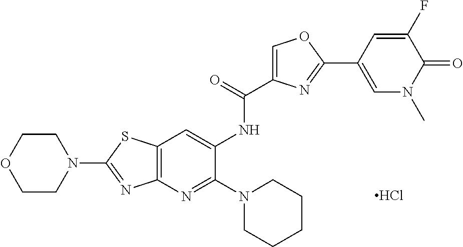 [SMILES: Cn1cc(-c2nc(C(=O)Nc3cc4sc(N5CCOCC5)nc4nc3N3CCCCC3)co2)cc(F)c1=O ; Cl] | ¹HNMR (400 MHz, DMSO-d₆): δ 9.60 (s, 1H), 8.89 (d, 1H), 8.42 (s, 1H), 7.76 (d, 1H), 3.70-3.57 (m, 8H), 3.10-2.95 (m, 4H), 2.46 (s, 3H), 1.77-1.58 (m, 6H). LCMS: m/z = 540.2 (M + 1)⁺; HPLC: 97.92%. |

### EXAMPLE 14

### N-(5-(5-methylpyridin-2-yl)-2-morpholinooxazolo[4,5-b]pyridin-6-yl)-2-(2-methylpyridin-4-yl)oxazole-4-carboxamide

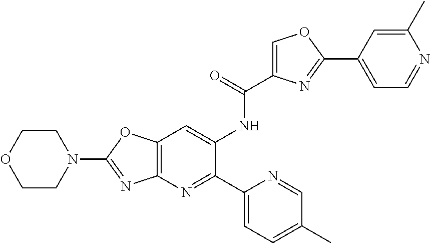 [SMILES: Cc1ccc(-c2nc3nc(N4CCOCC4)oc3cc2NC(=O)c2coc(-c3ccnc(C)c3)n2)nc1]

### Step 1: Preparation of 5-(5-methylpyridin-2-yl)-2-morpholino-6-nitrooxazolo[4,5-b]pyridine

In a sealed tube, 5-chloro-2-morpholino-6-nitrooxazolo[4,5-b]pyridine (1 g, 3.496 mmol), 5-methyl pyridine-2-boronic acid (718 mg, 5.244 mmol) and sodium carbonate (741 mg, 6.992 mmol) in 1,2-dimethoxyethane (15 mL) and water (3 mL) were taken and purged with argon for 10 min. To this reaction mixture Pd(dppf)Cl₂ (127 mg, 0.174 mmol) was added and heated at 95° C. overnight. The solvent was distilled out and compound was purified by 60-120 silica gel column chromatography using 5% methanol in DCM as eluent to obtain the title compound (200 mg

### Step 2: Preparation of5-(5-methylpyridin-2-yl)-2-morpholinooxazolo[4,5-b]pyridin-6-amine

Using the same reaction conditions as described in step 7 of Example 1, 5-(5-methylpyridin-2-yl)-2-morpholino-6-nitrooxazolo[4,5-b]pyridine (300 mg, 0.879 mmol) was reduced to afford the title compound (225 mg). LCMS: m/z=312.2 (M+1)(M+1)⁺.

### Step 3: Preparation of N-(5-(5-methylpyridin-2-yl)-2-morpholinooxazolo[4,5-b]pyridin-6-yl)-2-(2-methylpyridin-4-yl)oxazole-4-carboxamide

Using the same reaction conditions as described in step 8 of Example 1, 5-(5-methylpyridin-2-yl)-2-morpholinooxazolo[4,5-b]pyridin-6-amine (183 mg, 0.588 mmol) was coupled with intermediate 1 (100 mg, 0.490 mmol) to afford the title compound (7 mg).

¹HNMR (400 MHz, CDCl₃): δ 15.01 (s, 1H), 9.20 (s, 1H), 8.71-8.62 (m, 3H), 8.38 (s, 1H), 7.92 (s, 1H), 7.80-7.70 (dd, 2H), 3.83-3.78 (m, 8H), 2.71 (s, 3H), 2.44 (s, 3H). LCMS: m/z=498.40 (M+1)⁺; HPLC: 97.19%

### EXAMPLE 15

### N-(5-(3-hydroxy-3-(hydroxymethyl)piperidin-1-yl)-2-morpholinooxazolo[4,5-b]pyridin-6-yl)-2-(2-methylpyridin-4-yl)oxazole-4-carboxamide hydrochloride

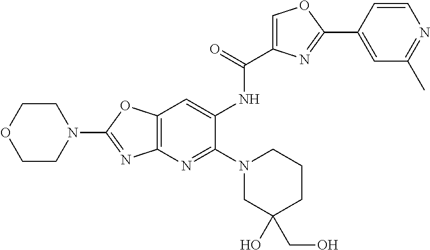 [SMILES: Cc1cc(-c2nc(C(=O)Nc3cc4oc(N5CCOCC5)nc4nc3N3CCCC(O)(CO)C3)co2)ccn1]

### Step 1: Preparation of 6-chloro-2-nitropyridin-3-ol

Potassium nitrate (14 g, 138.4 mmol) was added in several portions to a mixture of 2-chloropyridin-5-ol (10 g, 77.2 mmol) in concentrated sulphuric acid (50 ml) at 0° C. and further stirred at room temperature for 16 h. After the completion of reaction, reaction mixture was poured over crushed ice and the solid was filtered and dried to get the tittle compound (10.5 g, 78%). LCMS: m/z=173.3 (M+1)⁺.

### Step 2: Preparation of 2-amino-6-chloropyridin-3-ol

To a solution of 6-chloro-2-nitropyridin-3-ol (21 g, 126 mmol) in THF (250 ml) was added ammonium chloride (51.1 g, 965 mmol) in water (250 mL) and zinc dust (62.7 g, 965 mmol) and stirred at room temperature for 1 hr. The catalyst was filtered through Celite®, the filtrate was extracted with ethyl acetate and the organic layer was distilled out to obtain the title compound (13.3 g, 74.8%). LCMS: m/z=145.2 (M+1)⁺.

### Step 3: Preparation of 5-chlorooxazolo[4,5-b]pyridine-2-thiol

A solution of 2-amino-6-chloropyridin-3-ol (19.5 g, 135.4 mmol) and potassium ethyl xanthate (29.3 g, 182.8 mmol) in pyridine (150 mL) was heated at 110° C. overnight. The reaction mixture was cooled to 0° C. and diluted with ice water, acidified with conc. HCl, the solid was filtered and dried under vacuum to afford the title compound (35 gm, 69%). LCMS: m/z=184.8 (M+1)⁺

### Step 4: Preparation of 5-chloro-2-(methylthio)oxazolo[4,5-b]pyridine

To a stirred solution of 5-chlorooxazolo[4,5-b]pyridine-2-thiol (36 g, 193 mmol) in ethyl acetate (360 mL) was added potassium carbonate (53.42 g, 387 mmol) and methyl iodide (23.9 g, 387 mmol) and the reaction mixture was stirred at RT for 2 h. After completion of the reaction, the mixture was diluted with water and extracted with ethyl acetate; dried over sodium sulphate and concentrated to afford the title compound (32.5 g, 84.6%). LCMS: m/z=200.9 (M+1)⁺.

### Step 5: Preparation of 5-chloro-2-morpholinooxazolo[4,5-b]pyridine

To a solution of 5-chloro-2-(methylthio)oxazolo[4,5-b]pyridine (32 g, 160 mmol) in THF (320 mL) was added morpholine (65 mL) and heated at 75° C. overnight. The reaction mixture was concentrated and residue was diluted with water. The solid formed was filtered and dried to afford the title compound (32 g, 83.3%).

¹HNMR (400 MHz, DMSO-d₆): δ 7.81 (d, 1H), 7.07 (d, 1H), 3.74-3.64 (m, 8H). LCMS: m/z=240.0 (M+1)⁺.

### Step 6: Preparation of 5-chloro-2-morpholino-6-nitrooxazolo[4,5-b]pyridine

Using the same reaction conditions as described in step 1 of Example 1, 5-chloro-2-morpholinooxazolo[4,5-b]pyridine (23 g, 95 mmol) was nitrated to afford the title compound (20 mg, 73.2%). LCMS: m/z=284.9 (M+1)⁺.

### Step 7: Preparation of 3-(hydroxymethyl)-1-(2-morpholino-6-nitrooxazolo[4,5-b]pyridin-5-yl)piperidin-3-ol

To a solution of 5-chloro-2-morpholino-6-nitrooxazolo[4,5-b]pyridine (product of step-5 of example 15) (200 mg, 0.704 mmol) in DMF (2 mL) were added 3-(hydroxymethyl)piperidin-3-ol (110 mg, 0.845 mmol), potassium carbonate (145 mg, 1.056 mmol) and the reaction mixture was stirred at 80° C. for 3 h. Then the reaction mixture was quenched with ice water and extracted with ethyl acetate (2×10 mL), dried over sodium sulphate and distilled out the solvent to obtain the title compound (95 mg, 36%). LCMS: m/z=380.15 (M+1)⁺

### Step 8: Preparation of 1-(6-amino-2-morpholinooxazolo[4,5-b]pyridin-5-yl)-3-(hydroxymethyl)piperidin-3-ol

Using the same reaction conditions as described in step 7 of Example 1, 3-(hydroxymethyl)-1-(2-morpholino-6-nitrooxazolo[4,5-b]pyridin-5-yl)piperidin-3-ol (100 mg, 0.253 mmol) was reduced with zinc dust (130 mg, 2.03 mmol) and ammonium chloride (210 mg, 4.06 mmol) in THF/H₂O (5/5 mL) to get the title compound (80 mg, 87%). LCMS: m/z=350.20 (M+1)⁺

### Step 9: Preparation of N-(5-(3-hydroxy-3-(hydroxymethyl)piperidin-1-yl)-2-morpholinooxazolo[4,5-b]pyridin-6-yl)-2-(2-methylpyridin-4-yl)oxazole-4-carboxamide hydrochloride

Using the same reaction conditions as described in step 8 of Example 1, 1-(6-amino-2-morpholinooxazolo[4,5-b]pyridin-5-yl)-3-(hydroxymethyl)piperidin-3-ol (90 mg, 0.257 mmol) was reacted with intermediate 1 (52 mg, 0.257 mmol) to afford the title compound as free base. This was treated with methanol/methanolic HCL to afford title compound as hydrochloride salt (20 mg).

¹HNMR (400 MHz, DMSO-d₆): δ 9.93 (bs, 1H), 9.18 (s, 1H), 8.83(d, 1H), 8.60 (s, 1H), 8.22 (d, 1H), 8.06 (d, 1H), 3.80-3.50 (m, 11H),3.0-2.80(m, 4H), 2.71 (s, 3H), 2.0 (bs, 1H), 1.90-1.65 (m, 2H), 1.55-1.40 (m, 2H). LCMS: m/z=536.30 (M+1)⁺: HPLC: 97.87%

### EXAMPLE 16

### 2-(2-methylpyridin-4-yl)-N-(2-morpholino-5-(((1r,4r)-4-morpholinocyclohexyl)oxy)oxazolo[4,5-b]pyridin-6-yl)oxazole-4-carboxamide hydrochloride

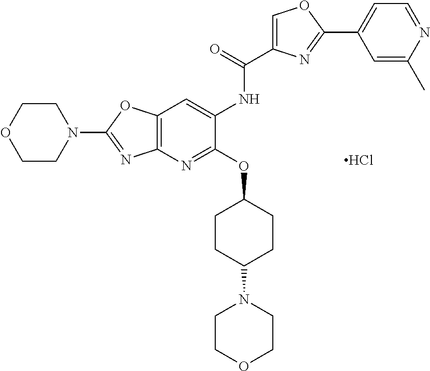

The title compound was prepared by procedure similar to the one described in example 15 with appropriate variations in reactants, quantities of reagents at suitable reaction conditions.

¹HNMR (400 MHz, DMSO-d₆): δ 8.93 (s, 1H), 8.71 (d, 1H), 8.31 (s, 1H), 8.11 (s, 1H), 8.02 (d, 1H), 7.64 (d, 1H), 4.90-4.80 (m, 1H), 4.09-4.00 (m, 4H), 3.69-3.58 (m, 8H), 3.41-3.38 (m, 2H), 3.13-3.06 (m, 2H), 2.67 (s, 3H), 2.32-2.16 (m, 4H), 1.70-1.52 (m, 4H). LCMS: m/z=590.1 (M+1)⁺: HPLC: 95.43%

### EXAMPLE 17

### (S)—N-(5-(3-fluoropyrrolidin-1-yl)-2-morpholinooxazolo[4,5-b]pyridin-6-yl)-2-(2-methylpyridin-4-yl)oxazole-4-carboxamide hydrochloride

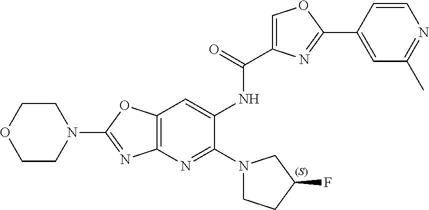

### Step-1: Preparation of (R)—N-(5-(3-hydroxypyrrolidin-1-yl)-2-morpholinooxazolo[4,5-b]pyridin-6-yl)-2-(2-methylpyridin-4-yl)oxazole-4-carboxamide

(R)—N-(5-(3-hydroxypyrrolidin-1-yl)-2-morpholinooxazolo[4,5-b]pyridin-6-yl)-2-(2-methylpyridin-4-yl)oxazole-4-carboxamide was prepared starting from 5-chloro-2-morpholino-6-nitrooxazolo[4,5-b]pyridine by using the similar reaction conditions described in the steps 7-9 of example 15 with appropriate variations in reactants, reagents and reaction conditions.

### Step-1: Preparation of (S)—N-(5-(3-fluoropyrrolidin-1-yl)-2-morpholinooxazolo[4,5-b]pyridin-6-yl)-2-(2-methylpyridin-4-yl)oxazole-4-carboxamide hydrochloride

DAST (57 mg, 0.356 mmol) was added to a solution of (R)—N-(5-(3-hydroxypyrrolidin-1-yl)-2-morpholinooxazolo[4,5-b]pyridin-6-yl)-2-(2-methylpyridin-4-yl)oxazole-4-carboxamide (100 mg, 0.203 mmol) in DCM (5 mL) at −78° C. After which the reaction mixture was allowed to stir at −10° C. over a period of 1 h. Then the reaction mixture was quenched with ice water, extracted with DCM and concentrated to obtain the crude compound. The crude compound was purified by prep. HPLC and treated with methanol/methanolic. HCl (2/2 mL) to afford title compound (25 mg, 23.5%).

¹HNMR (300 MHz, CD₃OD): δ 8.84 (s, 1H),8.80 (d, 1H), 8.49 (s, 1H), 8.40 (d, 1H), 7.92 (s, 1H), 5.40-5.10 (m, 1H), 3.74-3.72 (m, 12H), 2.81 (s, 3H), 2.30-2.05 (m, 2H). LCMS: m/z=494.3 (M+1). HPLC: 95.81%

### EXAMPLE 18

### (R)—N-(5-(3-fluoropyrrolidin-1-yl)-2-morpholinooxazolo[4,5-b]pyridin-6-yl)-2-(2-methylpyridin-4-yl)oxazole-4-carboxamide hydrochloride

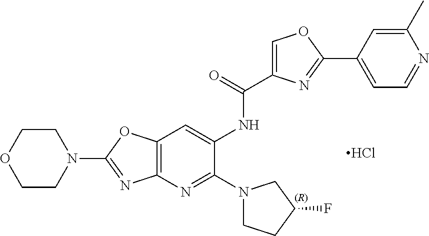

The title compound was prepared by procedure similar to the one described in Example 17 with appropriate variations in reactants, quantities of reagents at suitable reaction conditions.

1HNMR (400 MHz, CD₃OD): δ 8.92 (s, 1H),8.88 (d, 1H), 8.56 (s, 1H), 8.48 (d, 1H), 8.00 (s, 1H), 5.40-5.28 (m, 1H), 3.94-3.71 (m, 12H), 2.89 (s, 3H), 2.30-2.05 (m, 2H). LCMS: m/z=494.1 (M+1). HPLC: 95.72%.

### EXAMPLE 19

### N-(2-morpholino-5-(piperidin-1-yl)thiazolo[4,5-b]pyridin-6-yl)pyrazolo[1,5-a]pyrimidine-3-carboxamide

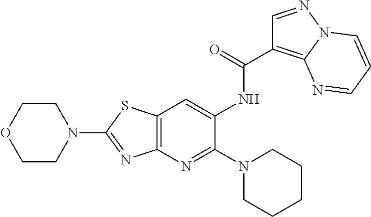 [SMILES: O=C(Nc1cc2sc(N3CCOCC3)nc2nc1N1CCCCC1)c1cnn2cccnc12]

### Step 1: Preparation of 3-bromo-6-chloropyridin-2-amine

To a solution of 2-amino-6-chloropyridine (15 g, 116 mmol) in chloroform (600 mL), was added a solution of bromine (4.2 g, 965 mmol) in chloroform (50 mL) at 0° C. and stirred at room temperature for 16 h. After the completion of reaction, the reaction mixture was quenched over ice cold water, extracted to DCM and concentrated. The crude was purified by silica gel column chromatography using 10% ethyl acetate in hexane as eluent to afford the title compound (6.2 g, 25.5%). LCMS: m/z=209.0 (M+1)⁺.

### Step 2: Preparation of 5-chlorothiazolo[4,5-b]pyridine-2-thiol

A solution of 3-bromo-6-chloropyridin-2-amine (42 g, 202 mmol) and potassium ethyl xanthate (58.15 g, 363 mmol) in DMF (200 mL) was heated at 150° C. for 4 h. The reaction mixture was cooled to 0° C., added into ice water and acidified with conc. HCl. The solid was filtered and dried under vacuum to afford the title compound (40 gm, 69%). LCMS: m/z=203.0 (M+1)⁺

### Step 3: Preparation of 5-chloro-2-(methylthio)thiazolo[4,5-b]pyridine

To a stirred solution of 5-chlorothiazolo[4,5-b]pyridine-2-thiol (37 g, 181.3 mmol) in ethyl acetate (200 mL), was added potassium carbonate (50 g, 362 mmol) and methyl iodide (38.9 g, 272 mmol) and stirred at RT for 2 h. After completion of reaction, the reaction mixture was diluted with water and extracted with ethyl acetate, dried over sodium sulphate and concentrated to afford the title compound (27 g, 70%). LCMS: m/z=217.6 (M+1)⁺

### Step 4: Preparation of 4-(5-chlorothiazolo[4,5-b]pyridin-2-yl)morpholine

To a solution of 5-chloro-2-(methylthio)thiazolo[4,5-b]pyridine (27 g, 125 mmol) in THF (100 mL), was added morpholine (50 mL) and heated at 75° C. overnight. After completion of reaction, reaction mixture was evaporated under reduced pressure. The residue was diluted with water and the solid was filtered and dried to afford the title compound (29 g, 91%).

¹HNMR (400 MHz, DMSO-d₆): δ 8.24 (d, 1H), 7.14 (d, 1H), 3.74-3.64 (m, 8H). LCMS: m/z=256.0 (M+1)⁺.

### Step 5: Preparation of 4-(5-chloro-6-nitrothiazolo[4,5-b]pyridin-2-yl)morpholine

Potassium nitrate (19.5 g, 192.8 mmol) was added in several portions to a mixture of 4-(5-chlorothiazolo[4,5-b]pyridin-2-yl)morpholine (29 g, 113.4 mmol) in concentrated sulphuric acid (100 ml) at 0° C. and stirred at room temperature for 16 h. After the completion of reaction, the reaction mixture was poured over crushed ice and the solid was filtered and dried to get the title compound (23 g, 68%).

¹HNMR (300 MHz, DMSO-d₆): δ 9.07 (s, 1H), 3.80-3.70 (m, 8H). LCMS: m/z=301.08 (M+1)⁺.

### Step 6: Preparation of 4-(6-nitro-5-(piperidin-1-yl)thiazolo[4,5-b]pyridin-2-yl)morpholine

Piperidine (30 ml) was added to a solution of 4-(5-chloro-6-nitrothiazolo[4,5-b]pyridin-2-yl)morpholine (14.0 g, 46.6 mmol) and stirred at 70° C. for 2 hours. The reaction mixture was concentrated and diluted with water. The solid was filtered and suction dried to get crude product which was then purified by silica gel column chromatography using DCM as eluent to get the title compound (14.4 g, 88%).

¹HNMR (300 MHz, CDCl₃): δ 8.44 (s, 1H), 3.90-3.70 (m, 8H), 3.50-3.40 (m, 4H), 1.75-1.65 (m, 6H). LCMS: m/z=350.20 (M+1)⁺.

### Step 7: Preparation of 2-morpholino-5-(piperidin-1-yl)thiazolo[4,5-b]pyridin-6-amine

To a solution of 4-(6-nitro-5-(piperidin-1-yl)thiazolo[4,5-b]pyridin-2-yl)morpholine (14.4 g, 41.14 mmol) in THF (220 ml) was added ammonium chloride (17.7 g, 329.14 mmol) in water (50 mL) and zinc dust (21.3 g, 329.14 mmol) and stirred at room temperature for 1 hr. The catalyst was filtered through Celite®; extracted with ethyl acetate and the solvent was distilled out to get the title compound (12.0 g, 91.6%).

¹HNMR (300 MHz, CDCl₃): 7.25 (d, 1H), 3.90-3.80 (m, 8H), 3.10-3.00 (m, 4H), 1.80-1.60 (m, 6H). LCMS: m/z=320.15 (M+1)⁺.

### Step 8: Preparation of N-(2-morpholino-5-(piperidin-1-yl)oxazolo[4,5-b]pyridin-6-yl)pyrazolo[1,5-a]pyrimidine-3-carboxamide

The solution of 2-morpholino-5-(piperidin-1-yl)thiazolo[4,5-b]pyridin-6-amine (70 mg, 0.218 mmol), pyrazolo[1,5-a]pyrimidine-3-carboxylic acid (39 mg, 0.240 mmol), HATU (124 mg, 0.327 mmol), DIPEA (113 mg, 0.872 mmol) in DMF(2 mL) was stirred at RT overnight. The reaction mixture was quenched with ice water and extracted the compound in ethyl acetate, dried over sodium sulphate and concentrated. The residual solid was triturated with diethyl ether, filtered and dried to afford the title compound (50 mg, 49.5%).

¹HNMR (300 MHz, CDCl₃): δ 10.49 (bs, 1H), 9.18 (s, 1H), 8.85-8.83 (dd, 1H), 8.78 (s, 1H), 8.76-8.74 (dd, 1H), 7.10-7.07 (m, 1H), 3.84-3.80 (m, 4H), 3.70-3.66 (m, 4H), 3.13-3.09 (m, 4H), 1.85-1.77 (m 4H), 1.64-1.62 (m, 2H). LCMS: m/z=465.25 (M+1)⁺; HPLC: 95.08%.

The below compounds were prepared by procedure similar to the one described in Example 19 with appropriate variations in reactants, quantities of reagents at suitable reaction conditions. The physicochemical characteristics of the compounds are summarized herein below table.

| Example | Structure | Analytical data |
|---|---|---|
| 20 | 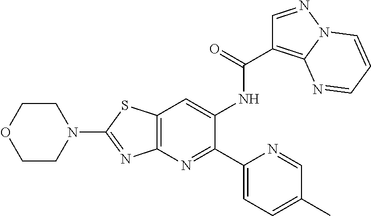 [SMILES: Cc1ccc(-c2nc3nc(N4CCOCC4)sc3cc2NC(=O)c2cnn3cccnc23)nc1] | ¹HNMR (400 MHz, CDCl₃): δ 13.80 (s, 1H), 9.46 (s, 1H), 8.84 (d, 2H), 8.79 (s, 1H), 8.61 (d, 1H), 8.45 (s, 1H), 7.69 (d, 1H), 7.08 (t, 1H), 3.88-3.75 (m, 8H), 2.42 (s, 3H). LCMS: m/z = 473.1 (M + 1)⁺; HPLC: 98.95%. |
| 21 | 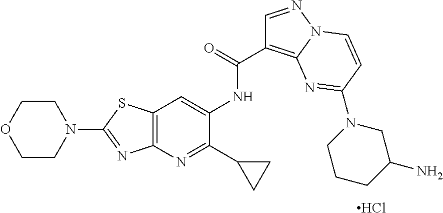 [SMILES: NC1CCCN(c2ccn3ncc(C(=O)Nc4cc5sc(N6CCOCC6)nc5nc4C4CC4)c3n2)C1 ; Cl] | ¹HNMR (300 MHz, CD₃OD): δ 8.90 (s, 1H), 8.65 (d, 1H), 8.38 (s, 1H), 6.91 (d, 1H), 4.50- 4.40 (m, 1H), 4.20-4.10 (m, 1H), 3.90-3.80 (m, 8H), 3.71-3.53 (m, 2H), 1.80-1.77 (m, 4H), 1.28-1.07 (m, 6H). LCMS: m/z = 520.1 (M + 1)⁺; HPLC: 97.57% |
| 22 | 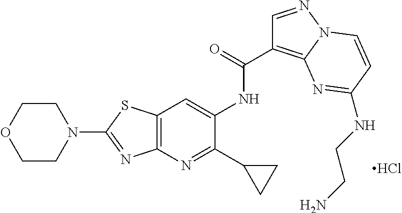 [SMILES: NCCNc1ccn2ncc(C(=O)Nc3cc4sc(N5CCOCC5)nc4nc3C3CC3)c2n1 ; Cl] | ¹HNMR (400 MHz, DMSO-d₆): δ 9.61 (s, 1H), 8.64 (d, 1H), 8.43 (bs, 1H), 8.33 (s, 1H), 8.30 (s, 1H), 8.03 (bs, 3H), 6.44 (d, 1H), 3.72- 3.58 (m, 8H), 3.55 (t, 2H), 3.03-3.02 (m, 2H), 2.28-2.23 (m, 1H), 0.97-0.92 (m, 4H). LCMS: m/z = 480.1 (M + 1)⁺; HPLC: 95.62% |
| 23 | 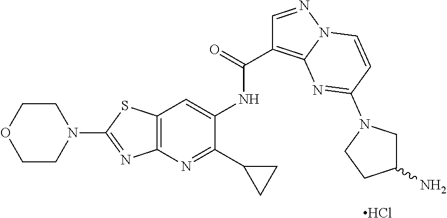 [SMILES: NC1CCN(c2ccn3ncc(C(=O)Nc4cc5sc(N6CCOCC6)nc5nc4C4CC4)c3n2)C1 ; Cl] | ¹HNMR (400 MHz, CD₃OD): δ 8.80 (s, 1H), 8.64 (d, 1H), 8.35 (s, 1H), 6.61 (d, 1H), 4.20- 4.10 (m, 1H), 3.90-3.75 (m, 8H), 2.60-2.40 (m, 4H), 1.40-1.06 (m, 7H). LCMS: m/z = 506.2 (M + 1)⁺; HPLC: 96.08% |

### EXAMPLE 24

### N-(2-morpholino-5-(piperidin-1-yl)oxazolo[4,5-b]pyridin-6-yl)pyrazolo[1,5-a]pyrimidine-3-carboxamide

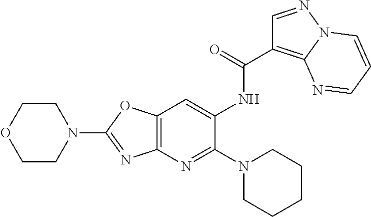 [SMILES: O=C(Nc1cc2oc(N3CCOCC3)nc2nc1N1CCCCC1)c1cnn2cccnc12]

### Step 1: Preparation of 6-chloro-2-nitropyridin-3-ol

Potassium nitrate (14 g, 138.4 mmol) was added in several portions to a mixture of 2-chloropyridin-5-ol (10 g, 77.2 mmol) in concentrated sulphuric acid (50 ml) at 0° C. and the reaction mixture was further stirred at room temperature for 16 h. After the completion of reaction, the reaction mixture was poured into the crushed ice; the solid was filtered and dried to obtain the title compound (10.5 g, 78%). LCMS: m/z=173.3 (M+1)⁺.

### Step 2: Preparation of 2-amino-6-chloropyridin-3-ol

To a solution of 6-chloro-2-nitropyridin-3-ol (21 g, 126 mmol) in THF (250 ml) was added ammonium chloride (51.1 g, 965 mmol) in water (250 mL) and zinc dust (62.7 g, 965 mmol) and the reaction mixture was stirred at room temperature for 1 h. The catalyst was filtered through Celite® and the filtrate was extracted with ethyl acetate and distilled out the solvent to get the title compound (13.3 g, 74.8%). LCMS: m/z=145.2 (M+1)⁺.

### Step 3: Preparation of 5-chlorooxazolo[4,5-b]pyridine-2-thiol

A solution of 2-amino-6-chloropyridin-3-ol (19.5 g, 135.4 mmol),potassium ethyl xanthate (29.3 g, 182.8 mmol) in pyridine (150 mL) was heated at 110° C. overnight. Then the reaction mixture was cooled to 0° C. and added ice water, acidified with Conc. HCl and the obtained was filtered, dried under vacuum to afford the title compound (35 gm, 69%). LCMS: m/z=184.8 (M+1)⁺

### Step 4: Preparation of 5-chloro-2-(methylthio)oxazolo[4,5-b]pyridine

To a stirred solution of 5-chlorooxazolo[4,5-b]pyridine-2-thiol (36 g, 193 mmol) in ethyl acetate (360 mL) was added potassium carbonate (53.42 g, 387 mmol) and methyl iodide (23.9 g, 387 mmol) and stirred at RT for 2 h. After completion of the reaction, the reaction mixture was diluted with water and extracted with ethyl acetate, dried over sodium sulphate and concentrated to afford the title compound (32.5 g, 84.6%). LCMS: m/z=200.9 (M+1)⁺

### Step 5: Preparation of 5-chloro-2-morpholinooxazolo[4,5-b]pyridine

To a solution of 5-chloro-2-(methylthio)oxazolo[4,5-b]pyridine (32 g, 160 mmol) in THF (320 mL) was added morpholine (65 mL) and heated at 75° C. overnight. Then the reaction mixture was concentrated to get the crude and the crude was diluted with water. The solid formed was filtered and dried to afford the title compound (32 g, 83.3%).

¹HNMR (400 MHz, DMSO-d₆): δ 7.81 (d, 1H), 7.07 (d, 1H), 3.74-3.64 (m, 8H). LCMS: m/z=240.0 (M+1)⁺.

### Step 6: Preparation of 5-chloro-2-morpholino-6-nitrooxazolo[4,5-b]pyridine

Using the same reaction conditions as described in step 1 of Example 1, 5-chloro-2-morpholinooxazolo[4,5-b]pyridine (23 g, 95 mmol) was nitrated to afford the title compound (20 mg, 73.2%). LCMS: m/z=284.9 (M+1)⁺.

### Step 7: Preparation of 2-morpholino-6-nitro-5-(piperidin-1-yl)oxazolo[4,5-b]pyridine

To a solution of 5-chloro-2-morpholino-6-nitrooxazolo[4,5-b]pyridine (30 mg, 0.1056 mmol) in THF (2 mL) was added piperidine (11 mg, 0.126 mmol) and the reaction mixture was stirred at RT overnight, which was quenched with ice water; extracted with ethyl acetate (2×10 mL); dried over sodium sulphate and distilled out the solvent to obtain the title compound (30 mg, 89%). LCMS: m/z=334.5 (M+1)⁺.

### Step 8: Preparation of 2-morpholino-5-(piperidin-1-yl)oxazolo[4,5-b]pyridin-6-amine

Using the same reaction conditions as described in step 7 of Example 1, 2-morpholino-6-nitro-5-(piperidin-1-yl)oxazolo[4,5-b]pyridine (300 mg, 0.900 mmol) was reduced with zinc dust (468 mg, 7.207 mmol) and ammonium chloride (389 mg, 7.207 mmol) in THF (5 mL) to get the title compound (260 mg, 96%). LCMS: m/z=304.1 (M+1)⁺.

### Step 9: Preparation of N-(2-morpholino-5-(piperidin-1-yl)oxazolo[4,5-b]pyridin-6-yl)pyrazolo[1,5-a]pyrimidine-3-carboxamide

Using the same reaction conditions as described in step 8 of Example 1, 2-morpholino-5-(piperidin-1-yl)oxazolo[4,5-b]pyridin-6-amine (93 mg, 0.306 mmol) was reacted with pyrazolo[1,5-a]pyrimidine-3-carboxylic acid (50 mg, 0.306 mmol) to afford the title compound (67 mg, 48.5%).

¹HNMR (300 MHz, CD₃OD): δ 9.13-9.10 (dd, J =1.5 Hz, 5.7 Hz, 1H), 8.88-8.86 (dd, J =1.5 Hz, 2.7 Hz, 1H), 8.80 (s, 1H), 8.68 (s, 1H), 7.30-7.26 (m, 1H), 3.83-3.80 (m, 4H), 3.72-3.69 (m, 4H), 3.03-2.99 (m, 4H), 1.85-1.84(m, 4H), 1.65 (m, 2H). LCMS: m/z=449.2 (M+1). HPLC: 99.77%

### EXAMPLE 25

### (R)—N-(5-(3-hydroxypyrrolidin-1-yl)-2-morpholinooxazolo[4,5-b]pyridin-6-yl)pyrazolo[1,5-a]pyrimidine-3-carboxamide

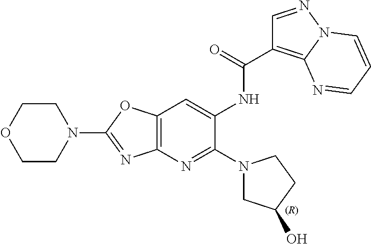

The title compound was prepared by procedure similar to the one described in Example 24 with appropriate variations in reactants, quantities of reagents at suitable reaction conditions

¹HNMR (CD₃OD, 300 MHz) δ: 9.16-9.13 (dd, J =1.8 Hz, 5.4 Hz, 1H), 8.87-8.85 (dd, J =1.5 Hz, 2.7 Hz, 1H), 8.67 (s, 1H), 8.21 (s, 1H), 7.28-7.25 (m, 1H), 4.47-4.45 (m, 1H), 3.82-3.79 (m, 4H), 3.76-3.67 (m, 6H), 3.40-3.35 (m, 2H), 2.18-2.11 (m, 2H), 1.93-1.91 (m, 2H). LCMS: m/z=451.2 (M+1)⁺; HPLC: 99.49%

The below compounds were prepared by procedure similar to the one described in Example 1 with appropriate variations in reactants, quantities of reagents at suitable reaction conditions. The physicochemical characteristics of the compounds are summarized herein below table.

| Example | Structure | Analytical data |
|---|---|---|
| 26 | 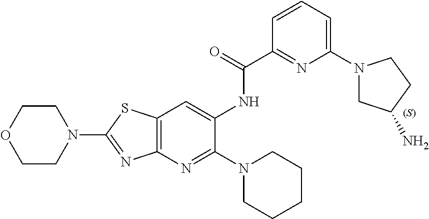 | ¹HNMR (300 MHz, CDCl₃): δ 10.60 (s, 1H), 9.11 (s, 1H), 7.63-7.54 (m, 2H), 6.57- 6.54 (d, 1H), 3.83-3.66 (m, 10H). 3.09-3.06 (m, 4H), 2.40-2.30 (m, 1H), 1.90-1.10 (m, 12H). LCMS: m/z = 509.20 (M + 1); HPLC: 95.96% |
| 27 | 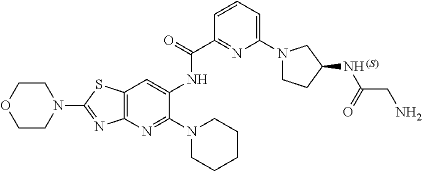 | ¹HNMR (300 MHz, CDCl₃): δ 10.60 (s, 1H), 9.11 (s, 1H), 7.68-7.58 (m, 3H), 6.59- 6.56 (d, 1H), 4.74-4.68 (m, 1H), 3.99-3.97 (m, 1H), 3.83-3.66 (m, 12H), 3.54-3.50 (m, 1H), 3.37 (s, 2H), 3.06 (m, 4H), 2.40-2.30 (m, 1H), 2.10-1.92 (m, 1H), 1.80-1.10 (m, 6H). LCMS: m/z = 566.4 (M + 1)⁺; HPLC: 97.51% |
| 28 | 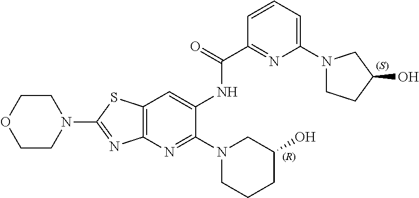 | ¹HNMR (400 MHz, CDCl₃): δ 10.40 (s, 1H), 9.04 (s, 1H), 7.66-7.53 (m, 2H), 6.60- 6.58 (d, 1H), 4.65 (s, 1H), 4.08 (s, 1H), 3.50-3.10 (m, 7H), 2.20-1.90 (m, 6H), 1.77 (s, 3H), 1.60-1.10 (m, 8H). LCMS: m/z = 526.0 (M + 1)⁺; HPLC: 97.13% |
| 29 | 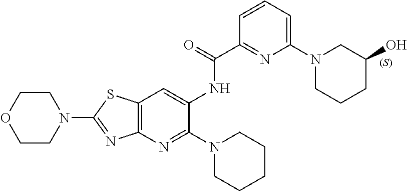 | ¹HNMR (400 MHz, CDCl₃): δ 10.45 (s, 1H), 9.04 (s, 1H), 7.69-7.62 (m, 2H), 6.90- 6.88 (d, 1H), 3.96-3.86 (m, 3H), 3.82-3.54 (m, 8H), 3.53-3.43 (m, 3H), 3.09-3.07 (m, 4H), 2.00-1.90 (m, 3H), 1.83-1.57 (m, 7H). LCMS: m/z = 524.3 (M + 1)⁺; HPLC: 97.57%. |
| 30 | 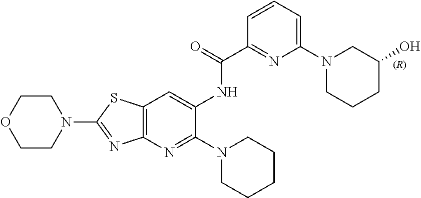 | 1HNMR (400 MHz, CDCl₃): δ 10.45 (s, 1H), 9.12 (s, 1H), 7.69-7.61 (m, 2H), 6.90- 6.88 (d, 1H), 3.96-3.86 (m, 3H), 3.82-3.67 (m, 8H), 3.55-3.42 (m, 2H), 3.09-3.06 (m, 4H), 2.00-1.58 (m, 11H). LCMS: m/z = 524.3 (M +1)⁺; HPLC: 99.33%. |
| 31 | 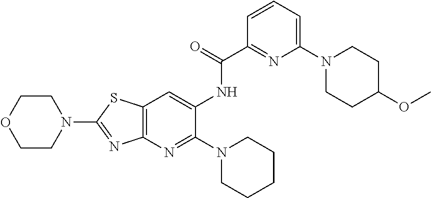 [SMILES: COC1CCN(c2cccc(C(=O)Nc3cc4sc(N5CCOCC5)nc4nc3N3CCCCC3)n2)CC1] | ¹HNMR (400 MHz, CDCl₃): δ 10.50 (s, 1H), 9.12 (s, 1H), 7.69-7.61 (m, 2H), 6.90- 6.88 (d, 1H), 4.20-4.10 (m, 2H), 3.84-3.80 (m, 4H), 3.70-3.67 (m, 4H), 3.55-3.45 (m, 1H), 3.42 (s, 3H), 3.39-3.38 (m, 2H), 3.09- 3.06 (m, 4H), 2.20-1.95 (m, 2H) 1.75-1.63 (m, 8H). LCMS: m/z = 538.3 (M + 1)⁺; HPLC: 95.49%. |
| 32 | 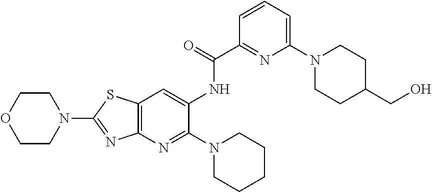 [SMILES: O=C(Nc1cc2sc(N3CCOCC3)nc2nc1N1CCCCC1)c1cccc(N2CCC(CO)CC2)n1] | 1HNMR (300 MHz, CDCl₃): δ 10.50 (s, 1H), 9.12 (s, 1H), 7.69-7.61 (m, 2H), 6.88- 6.85 (d, 1H), 4.59-4.54 (m, 2H), 3.84-3.80 (m, 4H), 3.70-3.67 (m, 4H), 3.60-3.54 (t, 2H), 3.09-3.06 (m, 4H), 2.97-2.90 (m, 2H), 1.93-1.76 (m, 8H), 1.40-1.25 (m, 6H). LCMS: m/z = 538.3 (M + 1)⁺; HPLC: 95.52%. |
| 33 | 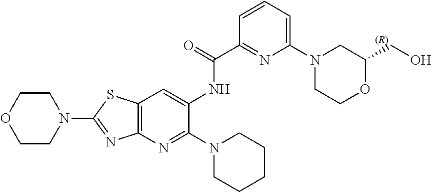 | ¹HNMR (400 MHz, CDCl₃): δ 10.50 (s, 1H), 9.12 (s, 1H), 7.69-7.61 (m, 2H), 6.88- 6.85 (d, 1H), 4.49-4.39 (m, 1H), 4.20-4.00 (m, 3), 3.84-3.67 (m, 14H), 3.20-3.00 (m, 4H), 1.73-1.71 (m, 4H), 1.56 (s, 2H). LCMS: m/z = 540.2 (M + 1)⁺; HPLC: 97.37%. |
| 34 | 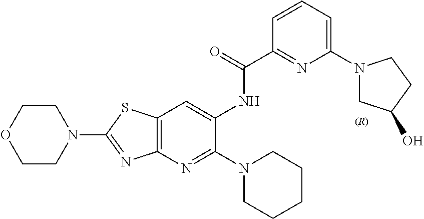 | ¹HNMR (400 MHz, CDCl₃): δ 10.50 (s, 1H), 9.12 (s, 1H), 7.67-7.56 (m, 2H), 6.59- 6.56 (d, 1H), 4.68 (s, 1H), 3.83-3.66 (m, 14H), 3.08 (s, 4H), 2.20-2.00 (m, 2H), 1.80- 1.62 (m, 5H). LCMS: m/z = 510.3 (M + 1); HPLC: 97.19%. |
| 35 | 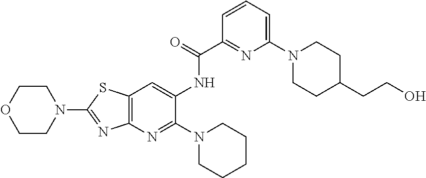 [SMILES: O=C(Nc1cc2sc(N3CCOCC3)nc2nc1N1CCCCC1)c1cccc(N2CCC(CCO)CC2)n1] | ¹HNMR (300 MHz, CDCl₃): δ 10.45 (s, 1H), 9.13 (s, 1H), 7.67-7.57 (m, 2H), 6.86- 6.83 (d, 1H), 4.54-4.49 (d, 2H), 3.83-3.66 (m, 11H), 3.09-3.05 (m, 4H), 2.96-2.87 (t, 2H), 1.89-1.72 (m, 7H), 1.55 (s, 2H), 1.34- 1.24 (m, 4H). LCMS: m/z = 552.3 (M + 1)⁺; HPLC: 95.95%. |
| 36 | 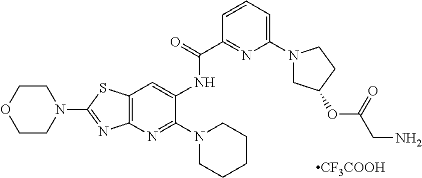 [SMILES: NCC(=O)O[C@H]1CCN(c2cccc(C(=O)Nc3cc4sc(N5CCOCC5)nc4nc3N3CCCCC3)n2)C1 ; "·CF3COOH"] | ¹HNMR (400 MHz, DMSO-d₆): δ 10.40 (s, 1H), 9.06 (s, 1H), 8.19 (bs, 2H), 7.78 (t, 1H), 7.45-7.43 (d, 1H), 6.84-6.81 (d, 1H), 5.58 (bs, 1H), 3.84-3.59 (m, 12H), 2.94 (bs, 4H), 2.38-2.36 (m, 4H), 1.72-1.58 (m, 6H). LCMS: m/z = 567.2 (M + 1)⁺; HPLC: 96.67% |
| 37 | 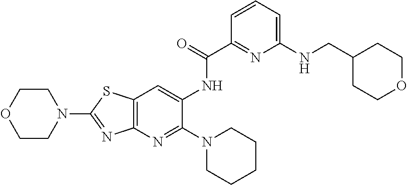 [SMILES: O=C(Nc1cc2sc(N3CCOCC3)nc2nc1N1CCCCC1)c1cccc(NCC2CCOCC2)n1] | ¹HNMR (400 MHz, DMSO-d₆): δ 10.2 (s, 1H), 9.00 (s, 1H), 7.57 (t, 1H), 7.31-7.30 (d, 1H), 6.90-6.78 (m, 2H), 3.8 (bs, 1H), 3.73 (t, 4H), 3.57 (t, 4H), 3.39-3.28 (m, 4H), 2.95 (t, 4H), 1.73-1.22 (m, 12H). LCMS: m/z = 538.1 (M + 1)⁺; HPLC: 95.49%. |
| 38 | 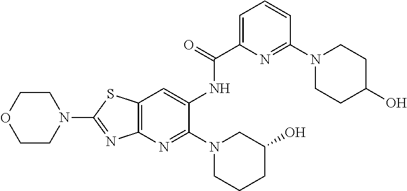 [SMILES: O=C(Nc1cc2sc(N3CCOCC3)nc2nc1N1CCC[C@@H](O)C1)c1cccc(N2CCC(O)CC2)n1] | ¹HNMR (400 MHz, DMSO-d₆): δ 10.40 (s, 1H) , 9.06 (s, 1H), 7.77-7.73 (m, 1H), 7.42-7.40 (d, 1H), 7.14-7.12 (d, 1H), 4.89- 4.87 (d, 1H), 4.82-4.81 (d, 1H), 4.16-4.11 (m, 2H), 3.76-3.56 (m, 8H), 3.27-2.66 (m, 6H), 2.00-1.22 (m, 10H). LCMS: m/z = 540.4 (M + 1)⁺; HPLC: 99.45%. |
| 39 | 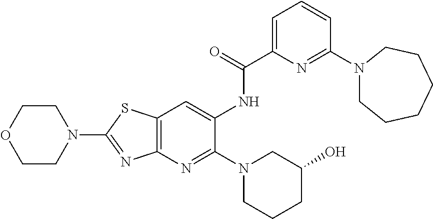 [SMILES: O=C(Nc1cc2sc(N3CCOCC3)nc2nc1N1CCC[C@@H](O)C1)c1cccc(N2CCCCCC2)n1] | ¹HNMR (400 MHz, DMSO-d₆): δ 10.20 (s, 1H), 9.00 (s, 1H), 7.72-7.68 (m, 1H), 7.35-7.34 (d, 1H), 6.93-6.91 (d, 1H), 4.88- 4.87 (d, 1H), 3.74-3.40 (m, 12H), 3.27-2.67 (m, 5H), 2.00-1.50 (m, 12H). LCMS: m/z = 538.4 (M + 1)⁺; HPLC: 98.58%. |
| 40 | 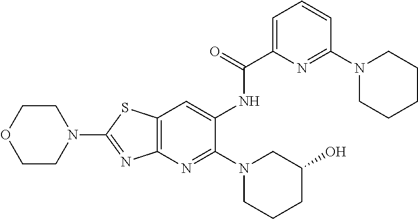 [SMILES: O=C(Nc1cc2sc(N3CCOCC3)nc2nc1N1CCC[C@@H](O)C1)c1cccc(N2CCCCC2)n1] | ¹HNMR (400 MHz, DMSO-d₆): δ 10.30 (s, 1H), 9.06 (s, 1H), 7.76-7.72 (m, 1H), 7.41-7.39 (d, 1H), 7.12-7.10 (d, 1H), 4.88- 4.87 (d, 1H), 3.75-3.56 (m, 12H), 3.19-2.66 (m, 5H), 2.00-1.62 (m, 10H). LCMS: m/z = 524.4 (M + 1)⁺; HPLC: 98.69%. |
| 41 | 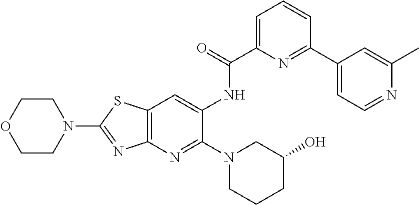 [SMILES: Cc1cc(-c2cccc(C(=O)Nc3cc4sc(N5CCOCC5)nc4nc3N3CCC[C@@H](O)C3)n2)ccn1] | ¹HNMR (400 MHz, DMSO-d₆): δ 10.60 (s, 1H), 9.08 (s, 1H), 8.65-8.64 (d, 1H), 8.39-8.37 (m, 1H), 8.27-8.25 (m, 2H), 8.06- 8.02 (m, 2H), 4.84-4.83 (d, 1H), 3.75-3.57 (m, 9H), 3.28-2.58 (m, 4H), 2.62 (s, 3H), 1.80-1.22 (m, 4H). LCMS: m/z = 532.3 (M + 1)⁺; HPLC: 98.54%. |
| 42 | 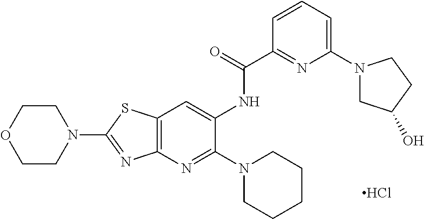 [SMILES: O=C(Nc1cc2sc(N3CCOCC3)nc2nc1N1CCCCC1)c1cccc(N2CC[C@H](O)C2)n1 ; Cl] | ¹HNMR (400 MHz, DMSO-d₆): δ 10.44 (s, 1H), 9.045 (s, 1H), 7.71 (t, 1H), 7.37-7.35 (d, 1H), 6.74-6.72 (d, 1H), 4.43 (bs, 1H), 3.74 (m, 4H), 3.70-3.65 (m, 7H), 2.95-2.92 (m, 4H), 2.08-2.05 (m, 2H), 1.95 (bs, 2H), 1.72 (bs, 4H), 1.56 (bs, 2H). LCMS: m/z = 510.3 (M + 1)⁺; HPLC: 97.38%. |
| 43 | 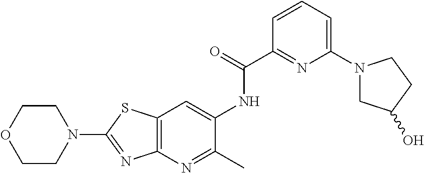 [SMILES: Cc1nc2nc(N3CCOCC3)sc2cc1NC(=O)c1cccc(N2CCC(O)C2)n1] | ¹HNMR (400 MHz, DMSO-d₆): δ 10.27 (s, 1H), 8.70 (s, 1H), 7.70 (t, 1H), 7.31-7.30 (d, 1H), 6.73-6.71 (d, 1H), 5.10-4.99 (m, 1H), 4.42 (bs, 1H), 3.75-3.72 (m, 4H), 3.59-3.56 (m, 6H), 3.49-3.40 (m, 2H), 2.52 (s, 3H), 2.06-2.03 (m, 1H), 1.94 (bs, 1H). LCMS: m/z = 441.3 (M + 1)⁺; HPLC: 96.86%. |
| 44 | 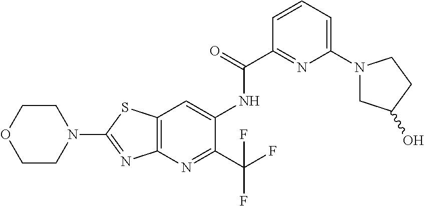 [SMILES: O=C(Nc1cc2sc(N3CCOCC3)nc2nc1C(F)(F)F)c1cccc(N2CCC(O)C2)n1] | ¹HNMR (400 MHz, DMSO-d₆): δ 10.58 (s, 1H), 9.13 (s, 1H), 7.73 (t, 1H), 7.34-732 (d, 1H), 6.77-6.75 (d, 1H), 5.05-5.04 (d, 1H), 4.43 (bs, 1H), 3.76-3.73 (m, 4H), 3.65-3.55 (m, 4H), 3.57-3.54 (m, 4H), 2.07-2.07 (m, 1H), 1.95 (bs, 1H). LCMS: m/z = 495.4 (M + 1)⁺; HPLC: 97.95%. |
| 45 | 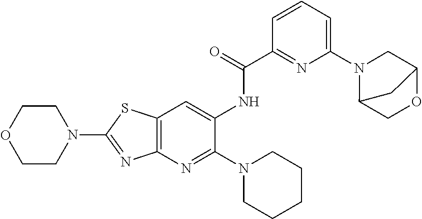 [SMILES: O=C(Nc1cc2sc(N3CCOCC3)nc2nc1N1CCCCC1)c1cccc(N2CC3CC2CO3)n1] | ¹HNMR (400 MHz, DMSO-d₆): δ 10.39 (s, 1H), 9.01 (s, 1H), 7.74 (t, 1H), 7.42-7.40 (d, 1H), 6.90-6.80 (d, 1H), 5.20 (bs, 1H), 4.75 (s, 1H), 3.82-3.81 (d, 1H), 3.75-3.70 (m, 4H), 3.58-3.56 (m, 4H), 3.48-3.41 (m, 1H), 2.94 (t, 4H), 1.94 (s, 2H), 1.70-1.51 (m, 5H), 1.22 (s, 3H). LCMS: m/z = 522.25 (M + 1)⁺; HPLC: 96.41% |
| 46 | 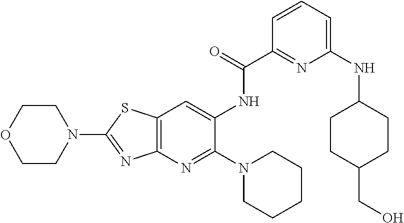 [SMILES: O=C(Nc1cc2sc(N3CCOCC3)nc2nc1N1CCCCC1)c1cccc(NC2CCC(CO)CC2)n1] | ¹HNMR (400 MHz, DMSO-d₆): δ 9.99 (s, 1H), 8.93 (s, 1H), 7.55 (t, 1H), 7.29-7.27 (d, 1H), 6.82-6.80 (d, 1H), 6.71-6.69 (d, 1H), 4.45 (t, 1H), 4.09-3.98 (bs, 1H), 3.75-3.72 (m, 4H), 3.58-3.56 (m, 4H), 2.97-2.67 (m, 3H), 2.02-1.99 (m, 3H), 1.83-1.74 (m, 5H), 1.57 (bs, 2H), 1.21-1.17 (m, 8H). LCMS: m/z = 552 (M + 1)⁺; HPLC: 95.36%. |
| 47 | 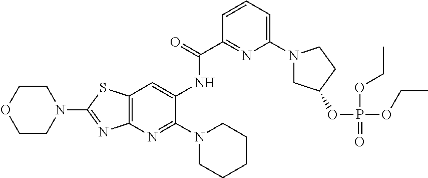 [SMILES: CCOP(=O)(OCC)O[C@H]1CCN(c2cccc(C(=O)Nc3cc4sc(N5CCOCC5)nc4nc3N3CCCCC3)n2)C1] | ¹HNMR (400 MHz, DMSO-d₆): δ 10.40 (s, 1H), 9.03 (s, 1H), 7.75 (t, 1H), 7.43-7.42 (d, 1H), 6.82-6.80 (d, 1H), 5.12 (bs, 1H), 4.14 (t, 1H), 4.04-3.98 (m, 4H), 3.90-3.82 (m, 1H), 3.73 (bs, 4H), 3.57 (bs, 4H), 2.94 (bs, 4H), 2.31-2.27 (m, 3H), 1.71 (bs, 4H), 1.57 (bs, 2H), 1.32-1.27 (m, 2H), 1.25-1.18 (m, 5H). LCMS: m/z = 646.4 (M + 1)⁺; HPLC: 95.41%. |
| 48 | 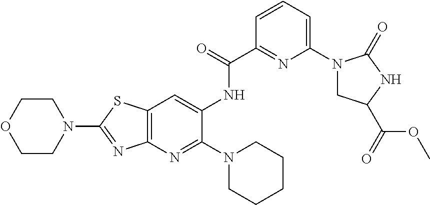 [SMILES: COC(=O)C1CN(c2cccc(C(=O)Nc3cc4sc(N5CCOCC5)nc4nc3N3CCCCC3)n2)C(=O)N1] | ¹HNMR (400 MHz, CDCl₃): δ 10.31 (s, 1H), 9.10 (s, 1H), 8.52-8.50 (d, 1H), 8.02- 7.99 (d, 1H), 7.88 (t, 1H), 5.30 (s, 1H), 4.67- 4.45 (m, 2H), 4.46-4.42 (m, 2H), 3.85-3.81 (m, 7H), 3.70-3.66 (m, 4H), 3.12-3.07 (m, 4H), 1.75 (bs, 4H). LCMS: m/z = 567.3 (M + 1)⁺; HPLC: 96.14%. |
| 49 | 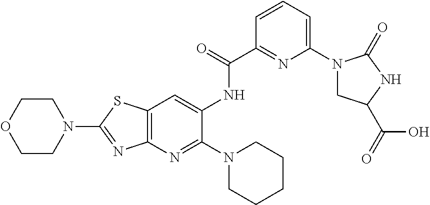 [SMILES: O=C(Nc1cc2sc(N3CCOCC3)nc2nc1N1CCCCC1)c1cccc(N2CC(C(=O)O)NC2=O)n1] | ¹HNMR (400 MHz, DMSO-d₆): δ 10.20 (s, 1H), 8.99 (s, 1H), 8.46-8.44 (d, 1H), 7.95 (t, 1H), 7.78-7.76 (d, 1H), 7.68-7.58 (m, 2H), 4.33 (bs, 2H), 4.41-4.40 (bs, 1H), 3.70-3.72 (m, 4H), 3.58-3.56 (m, 4H), 2.94 (bs, 4H), 1.70 (bs, 3H), 1.58 (bs, 3H). LCMS: m/z = 553.3 (M + 1)⁺; HPLC: 93.44%. |
| 50 | 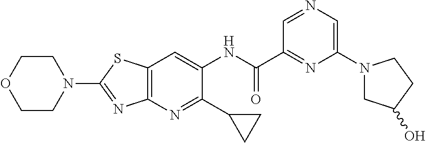 [SMILES: O=C(Nc1cc2sc(N3CCOCC3)nc2nc1C1CC1)c1cncc(N2CCC(O)C2)n1] | ¹HNMR (300 MHz, DMSO-d₆): δ 10.29 (s, 1H), 8.41 (s, 1H), 8.37 (s, 1H), 8.21 (s, 1H), 5.06-5.05 (d, 1H), 4.43 (m, 1H), 3.73- 3.70 (m, 4H), 3.70-3.40 (m, 6H), 2.17 (m, 1H), 2.10-1.90 (m, 4H), 0.96-0.92 (m, 4H). LCMS: m/z = 468.4 (M + 1)⁺; HPLC: 95.20% |
| 51 | 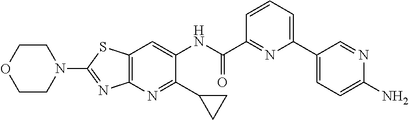 [SMILES: Nc1ccc(-c2cccc(C(=O)Nc3cc4sc(N5CCOCC5)nc4nc3C3CC3)n2)cn1] | ¹HNMR (400 MHz, DMSO-d₆): δ 10.69 (s, 1H), 8.95 (s, 1H), 8.75 (d, 1H), 8.28 (s, 1H), 8.17 (d, 1H), 8.11 (t, 1H), 8.05 (d, 1H), 7.5 (bs, 2H), 6.9 (d, 1H), 3.75-3.72 (m, 4H), 3.61-3.59 (m, 4H), 2.24 (m, 1H), 0.986-0.98 (m, 2H), 0.94-0.92 (m, 2H). LCMS: m/z = 474.3 (M + 1)⁺; HPLC: 99.08% |
| 52 | 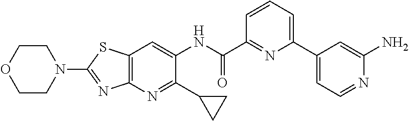 [SMILES: Nc1cc(-c2cccc(C(=O)Nc3cc4sc(N5CCOCC5)nc4nc3C3CC3)n2)ccn1] | ¹HNMR (400 MHz, DMSO-d₆): δ 10.60 (s, 1H), 8.38 (s, 1H), 8.17-8.13 (m, 3H), 8.07- 8.06 (d, 1H), 7.42-7.40 (dd, 1H), 7.26 (s, 1H), 6.02 (s, 2H), 3.74-3.72 (m, 4H); 3.60- 3.33 (m, 4H), 2.22 (m, 1H), 0.98-0.94 (m, 4H). LCMS: m/z = 474.0 (M + 1)⁺; HPLC: 98.70% |
| 53 | 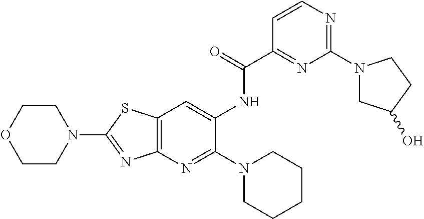 [SMILES: O=C(Nc1cc2sc(N3CCOCC3)nc2nc1N1CCCCC1)c1ccnc(N2CCC(O)C2)n1] | ¹HNMR (300 MHz, DMSO-d₆): δ 10.40 (s, 1H); 9.02 (s, 1H), 8.65-8.63 (d, 1H), 7.25- 7.23 (d, 1H), 5.05 (m, 1H), 4.42 (m, 1H), 3.73-3.70 (m, 4H), 3.70-3.56 (m, 6H), 2.92 (m, 4H), 2.07-1.94 (m, 4H), 1.72 (m, 4H), 1.56 (m, 2H). LCMS: m/z = 510.9 (M + 1)⁺; HPLC: 97.502% |
| 54 | 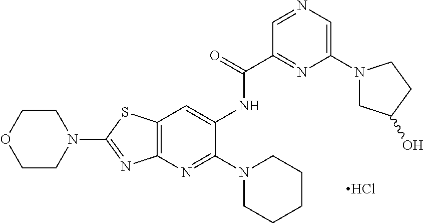 [SMILES: O=C(Nc1cc2sc(N3CCOCC3)nc2nc1N1CCCCC1)c1cncc(N2CCC(O)C2)n1 ; Cl] | ¹HNMR (400 MHz, CD₃OD): δ 8.92 (s, 1H), 8.52 (s, 1H), 8.29 (s, 1H), 4.65 (m, 1H), 3.90-3.88 (m, 4H), 3.82-3.78 (m, 6H), 3.70 (m, 2H), 3.35 (m, 2H), 2.30-2.10 (m, 4H), 1.86 (m, 4H), 1.74 (m, 2H). LCMS: m/z = 511.3 (M + 1)⁺; HPLC: 96.03% |
| 55 | 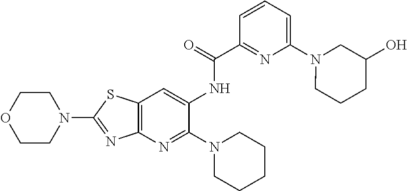 [SMILES: O=C(Nc1cc2sc(N3CCOCC3)nc2nc1N1CCCCC1)c1cccc(N2CCCC(O)C2)n1] | ¹HNMR (400 MHz, CDCl₃): δ 10.45 (s, 1H), 9.11 (s, 1H), 7.69-7.61 (m, 2H), 6.90- 6.88 (d, 1H), 3.96-3.81 (m, 6H), 3.68 (m, 4H), 3.53-3.41 (m, 2H), 3.07 (m, 4H), 2.00- 1.90 (m, 3H), 1.76 (m, 4H). LCMS: m/z = 524.1 (M + 1)⁺; HPLC: 95.00% |
| 56 | 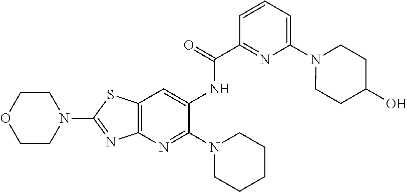 [SMILES: O=C(Nc1cc2sc(N3CCOCC3)nc2nc1N1CCCCC1)c1cccc(N2CCC(O)CC2)n1] | ¹HNMR (400 MHz, CDCl₃): δ 10.45 (s, 1H), 9.12 (s, 1H), 7.67-7.58 (m, 2H), 6.86- 6.84 (d, 1H), 4.24-4.23 (m, 2H), 3.98- 3.97 (m, 1H), 3.81-3.79 (m, 4H), 3.67- 3.65 (m, 4H), 3.33-3.26 (m, 2H), 3.07- 3.04 (m, 4H), 2.04-2.00 (m, 2H), 1.73- 1.72 (m, 4H). LCMS: m/z = 524.1 (M + 1)⁺; HPLC: 98.38% |
| 57 | 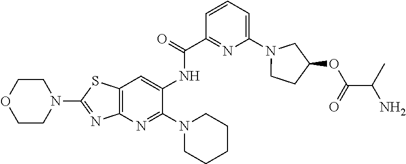 [SMILES: CC(N)C(=O)O[C@H]1CCN(c2cccc(C(=O)Nc3cc4sc(N5CCOCC5)nc4nc3N3CCCCC3)n2)C1] | ¹HNMR (400 MHz, CDCl₃): δ 10.45 (s, 1H), 9.11 (s, 1H), 7.69-7.61 (m, 2H), 6.59- 6.57 (d, 1H), 5.53 (m, 1H), 3.93-3.87 (m, 1H), 3.83-3.81 (m, 4H), 3.76-3.49 (m, 6H), 3.09-3.06 (m, 4H), 2.36-2.17 (m, 2H), 1.75-1.72 (m, 4H), 1.36-1.28 (m, 4H), 1.24 (s, 3H). LCMS: m/z = 581.3 (M + 1)⁺; HPLC: 95.39% |
| 58 | 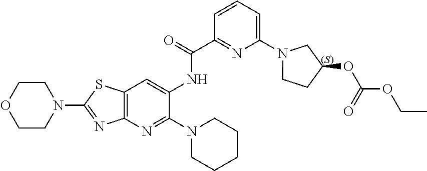 | ¹HNMR (400 MHz, CDCl₃): δ 10.45 (s, 1H), 9.10 (s, 1H), 7.65-7.59 (m, 2H), 6.58- 6.56 (d, 1H); 5.45 (m, 1H), 4.22-4.20 (q, 2H), 3.89-3.88 (m, 2H), 3.83-3.81 (m, 4H), 3.69-3.67 (m, 6H), 3.09-3.06 (m, 4H), 2.33 (m, 2H), 1.75-1.74 (m, 4H), 1.60 (m, 2H), 1.32-1.29 (t, 3H). LCMS: m/z = 582.4 (M + 1)⁺; HPLC: 95.07% |
| 59 | 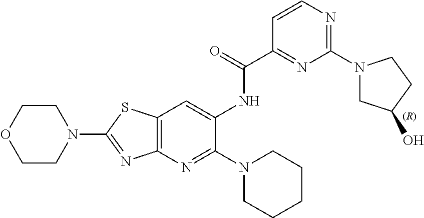 | ¹HNMR (300 MHz, DMSO-d₆): δ 10.45 (s, 1H), 9.04 (s, 1H), 8.67-8.65 (d, 1H), 7.27- 7.25 (d, 1H), 5.06 (m, 1H), 4.43 (m, 1H), 3.73-3.72 (m, 4H), 3.59-3.57 (m, 6H), 2.94 (m, 4H), 2.06-1.96 (m, 4H), 1.74 (m, 4H), 1.57 (m, 2H). LCMS: m/z = 511.4 (M + 1)⁺; HPLC: 96.00% |
| 60 | 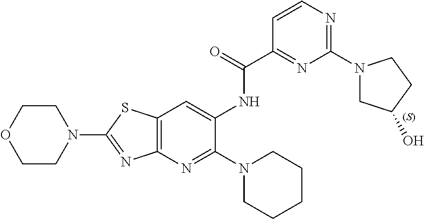 | ¹HNMR (400 MHz, DMSO-d₆): δ 10.45 (s, 1H), 9.04 (s, 1H), 8.66-8.65 (d, 1H), 7.27- 7.25 (d, 1H), 5.06 (m, 1H), 4.43 (m, 1H), 3.75-3.72 (m, 4H), 3.59-3.53 (m, 6H), 2.96-2.92 (m, 4H), 2.09-1.95 (m, 4H), 1.74 (m, 4H), 1.57 (m, 2H). LCMS: m/z = 511.4 (M + 1)⁺; HPLC: 98.67% |
| 61 | 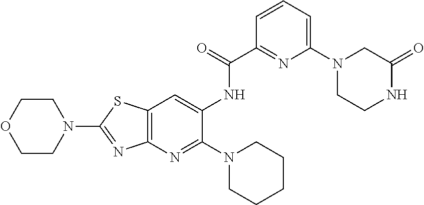 [SMILES: O=C1CN(c2cccc(C(=O)Nc3cc4sc(N5CCOCC5)nc4nc3N3CCCCC3)n2)CCN1] | ¹HNMR (300 MHz, CDCl₃): δ 10.45 (s, 1H), 9.12 (s, 1H), 7.80-7.72 (m, 2H), 6.81- 6.78 (dd, 1H), 6.20 (s, 1H), 4.22 (s, 2H), 4.15-4.12 (t, 2H), 3.84-3.81 (m, 4H), 3.70- 3.61 (m, 4H), 3.61-3.59 (m, 2H), 3.10- 3.06 (m, 4H), 1.74 (m, 6H). LCMS: m/z = 523.4 (M + 1)⁺; HPLC: 95.98% |
| 62 | 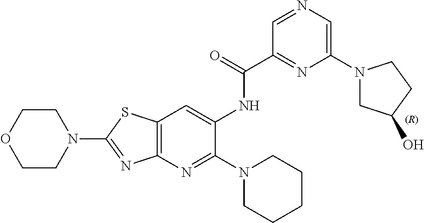 | ¹HNMR (400 MHz, DMSO-d₆): δ 10.30 (s, 1H), 9.04 (s, 1H), 8.46 (s, 1H), 8.27 (s, 1H), 5.13 (d, 1H), 4.47 (m, 1H), 3.74-3.56 (m, 10H), 2.95-2.92 (m, 4H), 2.14-1.94 (m, 4H), 1.72 (m, 4H), 1.58 (m, 2H). LCMS: m/z = 511.4 (M + 1)⁺; HPLC: 95.91% |
| 63 | 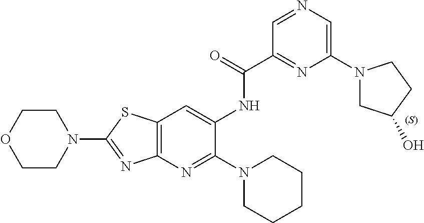 | ¹HNMR (400 MHz, DMSO-d₆): δ 10.30 (s, 1H), 9.04 (s, 1H), 8.46 (s, 1H), 8.26 (s, 1H), 5.12-5.11 (d, 1H), 4.47 (m, 1H), 3.73-3.57 (m, 10H), 2.94-2.93 (m, 4H), 2.14-1.94 (m, 4H), 1.72 (m, 4H), 1.57 (m, 2H). LCMS: m/z = 511.4 (M + 1)⁺; HPLC: 95.43% |
| 64 | 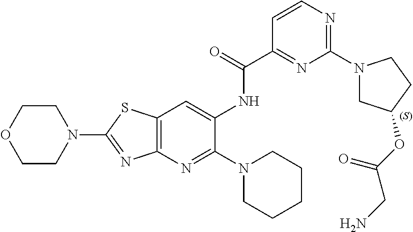 | ¹HNMR (400 MHz, CDCl₃): δ 10.45 (s, 1H), 9.07 (s, 1H), 8.58 (d, 1H), 7.42-7.40 (d, 1H), 5.55 (m, 1H), 3.90-3.62 (m, 10H), 3.05 (m, 4H), 2.40-2.20 (m, 4H), 1.74 (m, 4H), 1.59 (m, 2H), 1.14-1.13 (d, 2H). LCMS: m/z = 567.9 (M + 1)⁺; HPLC: 95.25% |
| 65 | 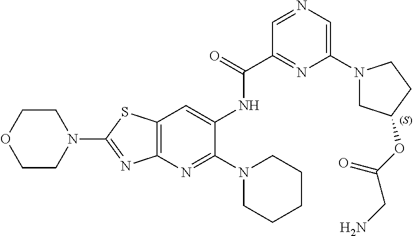 | ¹HNMR (400 MHz, CDCl₃): δ 10.38 (s, 1H), 9.12 (s, 1H), 8.77 (s, 1H), 8.11 (s, 1H), 5.58 (m, 1H), 3.90-3.62 (m, 10H), 3.08- 3.05 (m, 4H), 2.34-2.14 (m, 4H); 1.74 (m, 4H), 1.14-1.13 (d, 2H). LCMS: m/z = 568.3 (M + 1)⁺; HPLC: 96.42% |
| 66 | 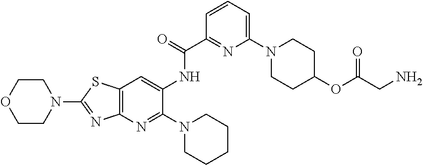 [SMILES: NCC(=O)OC1CCN(c2cccc(C(=O)Nc3cc4sc(N5CCOCC5)nc4nc3N3CCCCC3)n2)CC1] | ¹HNMR (400 MHz, CDCl₃): δ 10.50 (s, 1H), 9.13 (s, 1H), 7.71-7.63 (m, 2H), 6.89- 6.87 (d, 1H), 5.12 (m, 1H), 4.12-4.09 (m, 2H), 3.83-3.81 (m, 4H), 3.69-3.67 (m, 4H), 3.50-3.46 (m, 4H), 3.08-3.06 (m, 4H), 2.20-2.04 (m, 4H), 1.81-1.71 (m, 6H), 1.14-1.13 (d, 2H). LCMS: m/z = 581.5 (M + 1)⁺; HPLC: 96.74% |
| 67 | 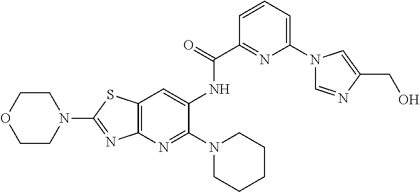 [SMILES: O=C(Nc1cc2sc(N3CCOCC3)nc2nc1N1CCCCC1)c1cccc(-n2cnc(CO)c2)n1] | ¹HNMR (400 MHz, CDCl₃): δ 10.62 (s, 1H), 9.13 (s, 1H), 8.60 (s, 1H), 8.29-8.27 (d, 1H), 8.13-8.09 (t, 1H), 7.84 (s, 1H), 7.63-7.62 (d, 1H), 4.74 (s, 2H), 3.84-3.82 (m, 4H), 3.71-3.69 (m, 4H), 3.12-3.09 (m, 4H), 1.79-1.77 (m, 4H), 1.63-1.62 (m, 2H). LCMS: m/z = 521.1 (M + 1)⁺; HPLC: 96.06% |
| 68 | 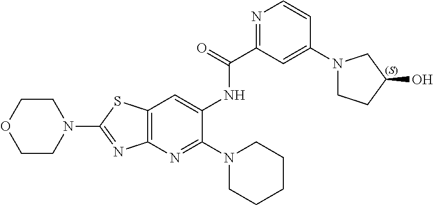 | ¹HNMR (400 MHz, DMSO-d₆): δ 10.65 (s, 1H), 9.03 (s, 1H), 8.23-8.21 (d, 1H), 7.20 (s, 1H), 6.65-6.64 (d, 1H), 5.08-5.07 (d, 1H), 4.43 (m, 1H), 3.73 (m, 4H), 3.56 (m, 4H), 3.51-3.42 (m, 1H), 3.22-3.20 (m, 1H); 2.97 (m, 4H), 2.10-1.90 (m, 4H), 1.77 (m, 4H), 1.58 (m, 2H). LCMS: m/z = 510.0 (M + 1)⁺; HPLC: 97.73% |
| 69 | 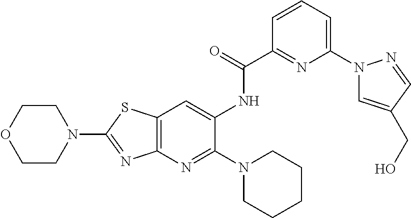 [SMILES: O=C(Nc1cc2sc(N3CCOCC3)nc2nc1N1CCCCC1)c1cccc(-n2cc(CO)cn2)n1] | ¹HNMR (300 MHz, CDCl₃): δ 10.65 (s, 1H), 9.15 (s, 1H), 8.83 (s, 1H), 8.21-8.03 (m, 2H), 7.82 (s, 1H), 7.22 (m, 1H), 4.74 (s, 2H), 3.83-3.81 (m, 4H), 3.75-3.68 (m, 4H), 3.11-3.09 (m, 4H), 1.80 (m, 4H), 1.63- 1.62 (m, 2H). LCMS: m/z = 521.15 (M + 1)⁺; HPLC: 95.21% |
| 70 | 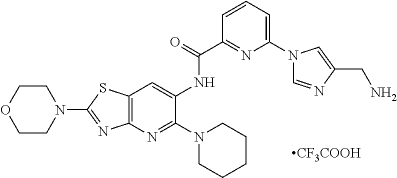 [SMILES: NCc1cn(-c2cccc(C(=O)Nc3cc4sc(N5CCOCC5)nc4nc3N3CCCCC3)n2)cn1 ; "·CF3COOH"] | ¹HNMR (300 MHz, CD₃OD): δ 8.938 (s, 1H), 8.87 (s, 1H), 8.27-8.25 (m, 2H), 8.16 (s, 1H), 7.97-7.95 (m, 1H), 4.17 (s, 2H), 3.82-3.81 (m, 4H), 3.65 (m, 4H), 3.13- 3.11 (m, 4H); 1.77 (m, 4H), 1.61 (m, 2H). LCMS: m/z = 520.3 (M + 1)⁺; HPLC: 93.16% |
| 71 | 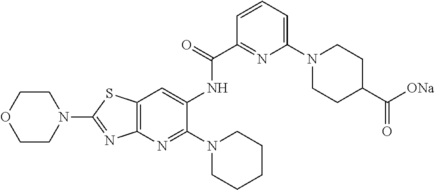 | ¹HNMR (400 MHz, DMSO-d₆): δ 8.90 (s, 1H), 7.72-7.68 (m, 1H), 7.37-7.35 (d, 1H), 7.05-7.03 (d, 1H), 4.36-4.33 (m, 2H), 3.70-3.69 (m, 4H), 3.54-3.53 (m, 4H), 2.89-2.86 (m, 6H), 2.15-2.12 (m, 1H), 1.85-1.82 (m, 2H), 1.65 (m, 4H), 1.52- 1.46 (m, 4H). LCMS: m/z = 552.25 (M − 23); HPLC: 97.32% |
| 72 | 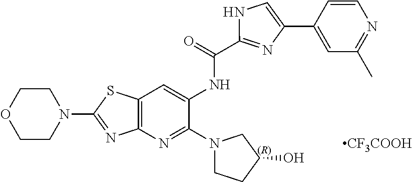 | ¹HNMR (300 MHz, CDCl₃): δ 8.51-8.48 (d, 1H); 8.20 (s, 1H), 8.02-8.00 (m, 2H), 7.96 (s, 1H), 4.42 (m, 1H), 3.80-3.55 (m, 10H), 3.47-3.41 (m, 2H), 2.71 (s, 3H), 2.02-1.91 (m, 2H). LCMS: m/z = 507.35 (M + 1)⁺; HPLC: 95.61% |
| 73 | 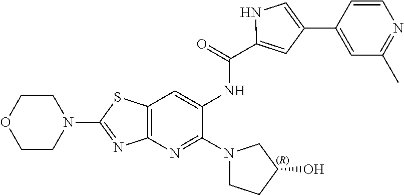 | ¹HNMR (400 MHz, CDCl₃): δ 9.72 (s, 1H), 8.55 (s, 1H), 8.44-8.43 (d, 1H), 8.22 (s, 1H), 7.04 (s, 1H), 7.28 (s, 1H), 7.24-7.22 (d, 1H), 7.17 (s, 1H), 4.60 (m, 1H), 3.83- 3.81 (m, 4H), 3.68-3.51 (m, 8H), 2.57 (s, 3H), 2.30-2.20 (m, 1H), 2.05-1.95 (m, 1H). LCMS: m/z = 506.1 (M + 1)⁺; HPLC: 88.60% |
| 74 | 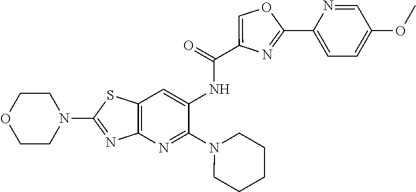 [SMILES: COc1ccc(-c2nc(C(=O)Nc3cc4sc(N5CCOCC5)nc4nc3N3CCCCC3)co2)nc1] | ¹HNMR (400 MHz, DMSO-d₆): δ 9.66 (s, 1H), 8.95 (s, 2H), 8.46 (s, 1H), 8.14-8.11 (d, 1H), 7.72-7.70 (d, 1H), 3.93 (s, 3H), 3.73 (m, 4H), 3.58 (m, 4H), 3.00 (m, 4H), 1.82 (m, 4H), 1.64 (m, 2H). LCMS: m/z = 522.3 (M + 1)⁺; HPLC: 96.92% |
| 75 | 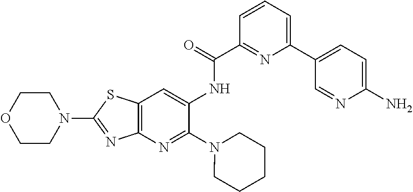 [SMILES: Nc1ccc(-c2cccc(C(=O)Nc3cc4sc(N5CCOCC5)nc4nc3N3CCCCC3)n2)cn1] | ¹HNMR (400 MHz, CDCl₃): δ 10.94 (s, 1H), 9.19 (s, 1H), 8.77 (d, 1H), 8.49 (dd, 1H), 8.20 (d, 1H), 7.90 (t, 1H), 7.86 (d, 1H), 6.66 (d, 1H), 4.92 (s, 2H), 3.84-3.82 (m, 4H), 3.71-3.68 (m, 4H), 3.12-3.09 (m, 4H), 1.76-1.73 (m, 4H), 1.70-1.65 (m, 2H). LCMS: 517.1 (M + 1)⁺; HPLC: 97.50% |
| 76 | 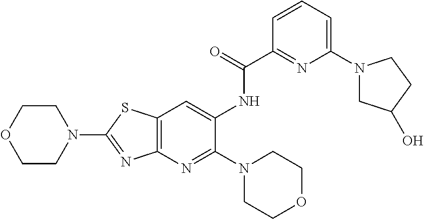 [SMILES: O=C(Nc1cc2sc(N3CCOCC3)nc2nc1N1CCOCC1)c1cccc(N2CCC(O)C2)n1] | ¹HNMR (400 MHz, DMSO-d₆): δ 10.37 (s, 1H), 9.06 (s, 1H), 7.72-7.70 (m, 1H), 7.37 (d, 1H), 6.74 (d, 1H), 5.07-5.06 (m, 1H), 4.49 (s, 1H), 3.80-3.58 (m, 16H), 3.03- 2.98 (m, 4H), 2.12-2.08 (m, 2H), LCMS: m/z = 512.5 (M + 1)⁺; HPLC: 95.22% |
| 77 | 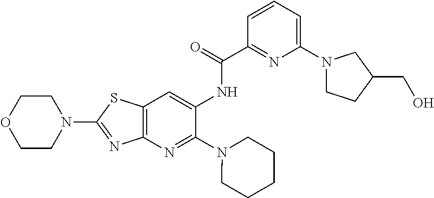 [SMILES: O=C(Nc1cc2sc(N3CCOCC3)nc2nc1N1CCCCC1)c1cccc(N2CCC(CO)C2)n1] | ¹HNMR (400 MHZ, DMSO-d₆): δ 10.45 (s, 1H), 9.05 (s, 1H), 7.73-7.69 (t, 1H), 7.38- 7.36 (d, 1H), 6.74-6.72 (d, 1H), 4.76-4.74 (m, 1H), 3.74-3.72 (m, 5H), 3.58-3.56 (m, 5H), 3.48-3.40 (m, 3H), 2.94-2.91 (m, 4H), 2.10-2.00 (m, 2H), 1.80-1.74 (m, 6H), 1.57- 1.56 (m, 2H). LCMS: 524.2 (M + 1)⁺; HPLC: 95.15%. |
| 78 | 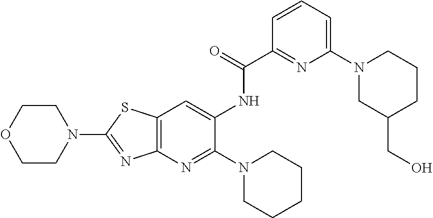 [SMILES: O=C(Nc1cc2sc(N3CCOCC3)nc2nc1N1CCCCC1)c1cccc(N2CCCC(CO)C2)n1] | ¹HNMR (300 MHz, CDCl₃): δ 10.45 (s, 1H), 9.10 (s, 1H), 7.70-7.50 (m, 2H), 6.92- 6.80 (m, 1H), 4.55-4.51 (m, 1H), 4.14-4.09 (m, 1H), 3.84-3.80 (m, 5H), 3.70-3.60 (m, 8H), 3.17-2.95 (m, 7H), 1.90-1.62 (m, 8H). LCMS: m/z = 538.2 (M + 1)⁺; HPLC: 96.21% |
| 79 | 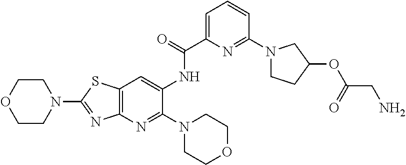 [SMILES: NCC(=O)OC1CCN(c2cccc(C(=O)Nc3cc4sc(N5CCOCC5)nc4nc3N3CCOCC3)n2)C1] | ¹HNMR (400 MHz, CDCl₃): δ 10.40 (s, 1H), 9.08 (s, 1H), 7.67-7.60 (m, 2H), 6.61- 6.59 (d, 1H), 5.58 (s, 2H), 3.89-3.81 (m, 11H), 3.70-3.68 (m, 5H), 3.45 (s, 2H), 3.19- 3.16 (m, 4H), 2.40-2.20 (m, 3H). LCMS: m/z = 569.2 (M + 1)⁺; HPLC: 95.31% |
| 80 | 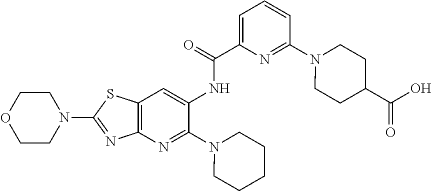 [SMILES: O=C(Nc1cc2sc(N3CCOCC3)nc2nc1N1CCCCC1)c1cccc(N2CCC(C(=O)O)CC2)n1] | ¹HNMR (400 MHz, CDCl₃): δ 10.60 (s, 1H), 9.13 (s, 1H), 7.67-7.65 (m, 2H), 6.88- 6.86 (d, 1H), 4.44-4.41 (m, 2H), 3.83-3.81 (m, 4H), 3.73-3.67 (m, 4H), 3.14-3.04 (m, 6H), 2.66-2.63 (m, 1H), 2.12-2.10 (m, 2H), 1.89-1.81 (m, 2H), 1.75-1.74 (m, 4H), 1.62- 1.61 (m, 2H). LCMS: m/z = 552.2 (M + 1)⁺; HPLC: 95.70%. |
| 81 | 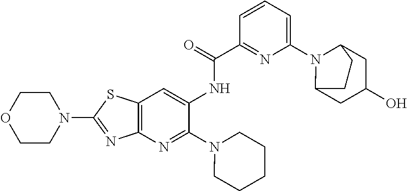 [SMILES: O=C(Nc1cc2sc(N3CCOCC3)nc2nc1N1CCCCC1)c1cccc(N2C3CCC2CC(O)C3)n1] | ¹HNMR (400 MHz, CDCl₃): δ 10.60 (s, 1H), 9.11 (s, 1H), 7.70-7.50 (m, 2H), 6.75- 6.70 (m, 1H), 4.80-4.60 (m, 1H), 4.30-4.10 (m, 1H), 3.83-3.81 (m, 4H), 3.69-3.64 (m, 4H), 3.09-3.06 (m, 4H), 2.40-2.35 (m, 1H), 2.20-2.00 (m, 4H), 1.85-1.70 (m, 6H), 1.65- 1.50 (m, 5H). LCMS: m/z = 550.2 (M + 1)⁺; HPLC: 94.90%. |
| 82 | 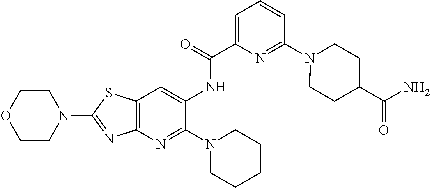 [SMILES: NC(=O)C1CCN(c2cccc(C(=O)Nc3cc4sc(N5CCOCC5)nc4nc3N3CCCCC3)n2)CC1] | ¹HNMR (400 MHz, CDCl₃): δ 10.50 (s, 1H), 9.13 (s, 1H), 7.70-7.62 (m, 2H), 6.88- 6.86 (d, 1H), 5.52-5.40 (m, 3H), 4.58-4.54 (m, 2H), 3.83-3.81 (m, 4H), 3.69-3.67 (m, 4H), 3.08-3.00 (m, 6H), 2.48-2.42 (m, 1H), 2.10-2.00 (m, 3H), 1.90-1.70 (m, 6H). LCMS: m/z = 551.3 (M + 1)⁺; HPLC: 95.15%. |
| 83 | 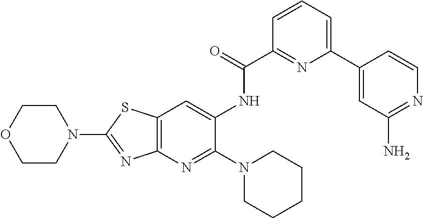 [SMILES: Nc1cc(-c2cccc(C(=O)Nc3cc4sc(N5CCOCC5)nc4nc3N3CCCCC3)n2)ccn1] | ¹HNMR (400 MHz, CDCl₃): δ 10.95 (s, 1H), 9.19 (s, 1H), 8.35-8.33 (d, 1H), 8.24- 8.23 (m, 1H), 8.06-8.02 (t, 1H), 7.93-7.91 (d, 1H), 7.42-7.41 (d, 1H), 7.22 (s, 1H), 4.16 (s, 2H), 3.84-3.81 (m, 4H), 3.70-3.68 (m, 4H), 3.11-3.06 (m, 4H), 1.79-1.73 (m, 4H), 1.57-1.56 (m, 2H). LCMS: m/z = 517.4 (M + 1)⁺; HPLC: 95.35%. |
| 84 | 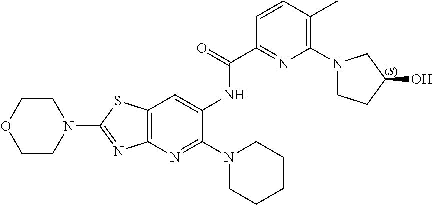 | ¹HNMR (400 MHz, CDCl₃): δ 9.26 (s, 1H), 8.25 (s, 1H), 6.70 (s, 1H), 6.64-6.24 (d, 1H), 3.76 (s, 1H), 3.30-3.15 (m, 2H), 3.00- 2.90 (m, 4H), 2.85-2.70 (m, 6H), 2.30-2.10 (m, 4H), 1.77 (s, 1H), 1.40-1.20 (m, 2H), 1.00-0.80 (m, 4H), 1.75-1.60 (m, 5H). LCMS: 524.3 (M + 1)⁺; HPLC: 95.46%. |
| 85 | 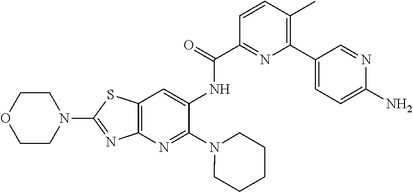 [SMILES: Cc1ccc(C(=O)Nc2cc3sc(N4CCOCC4)nc3nc2N2CCCCC2)nc1-c1ccc(N)nc1] | ¹HNMR (400 MHz, CDCl₃): δ 10.92 (s, 1H), 9.18 (s, 1H), 8.372-8.371 (d, 1H), 8.14-8.12 (d, 1H), 7.84-7.78 (m, 2H), 6.64- 6.62 (d, 1H), 4.64 (s, 2H), 3.83-3.80 (m, 4H), 3.69-3.66 (m, 4H), 3.03-3.00 (m, 4H), 2.50 (s, 3H) 1.60-1.55 (m, 4H), 1.41-1.39 (m, 2H). LCMS: m/z = 531.3 (M + 1)⁺; HPLC: 97.11%. |

The below compounds were prepared by procedure similar to the one described in Example 15 with appropriate variations in reactants, quantities of reagents at suitable reaction conditions. The physicochemical characteristics of the compounds are summarized herein below table.

| Example | Structure | Analytical data |
|---|---|---|
| 86 | 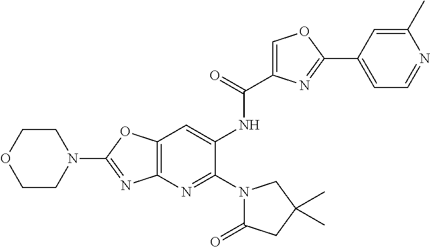 [SMILES: Cc1cc(-c2nc(C(=O)Nc3cc4oc(N5CCOCC5)nc4nc3N3CC(C)(C)CC3=O)co2)ccn1] | ¹HNMR (300 MHz, CDCl₃): δ 10.09 (s, 1H), 8.68-8.66 (d, 1H), 8.36 (s, 1H), 8.18 (s, 1H), 7.81-7.75 (d, 2H), 3.88-3.77 (m, 10H), 2.67 (s, 3H), 2.53 (s, 2H), 1.25 (s, 6H). LCMS: m/z = 518.0 (M + 1)⁺; HPLC: 98.71% |
| 87 | 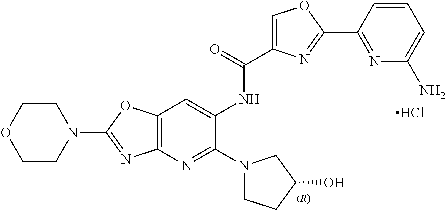 | ¹HNMR (300 MHz, CD₃OD): δ 8.86 (s, 1H), 8.09-8.06 (m, 1H), 8.01 (s, 1H), 7.63- 7.61 (d, 1H), 7.21-7.18 (d, 1H), 4.49 (s, 1H), 3.90-3.70 (m, 10H), 3.69-3.63 (m, 1H), 3.54-3.50 (m, 1H), 2.15-2.03 (m, 2H). LCMS: m/z = 493.30 (M + 1)⁺; HPLC 98.84% |
| 88 | 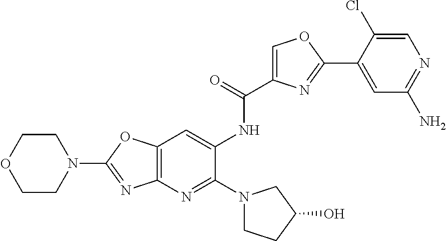 [SMILES: Nc1cc(-c2nc(C(=O)Nc3cc4oc(N5CCOCC5)nc4nc3N3CC[C@@H](O)C3)co2)c(Cl)cn1] | ¹HNMR (400 MHz, DMSO-d₆): δ 9.77 (s, 1H), 9.04 (s, 1H), 8.22 (s, 1H), 7.77 (s, 1H), 7.24 (s, 1H), 4.29 (s, 1H), 3.76-3.60 (m, 12H), 3.50-3.30 (m, 2H), 3.25-3.20 (m, 1H), 2.20-2.00 (m, 2H). LCMS: m/z = 527.3 (M + 1)⁺; HPLC: 95.88% |
| 89 | 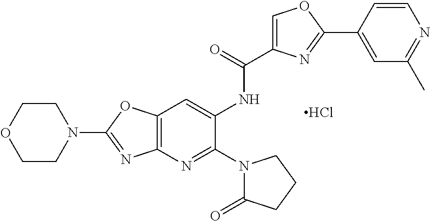 [SMILES: Cc1cc(-c2nc(C(=O)Nc3cc4oc(N5CCOCC5)nc4nc3N3CCCC3=O)co2)ccn1 ; Cl] | ¹HNMR (300 MHz, CD₃OD): δ 8.67 (s, 1H), 8.63-8.61 (d, 1H), 8.15 (s, 1H), 8.01 (s, 1H), 7.91-7.90 (d, 2H), 4.15-4.10 (t, 2H), 3.84-3.77 (m, 8H), 2.73-2.65 (m, 5H), 2.30-2.20 (q, 2H). LCMS: m/z = 490.3 (M + 1)⁺; HPLC: 98.07% |
| 90 | 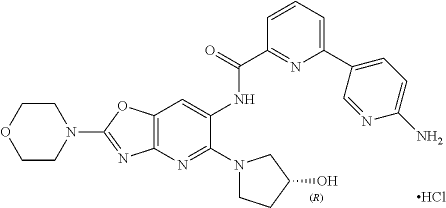 | ¹HNMR (400 MHz, DMSO-d₆): δ 10.53 (s, 1H), 9.08 (s, 1H), 8.94-8.92 (d, 1H), 8.40 (bs, 2H), 8.24-8.07 (m, 3H), 7.80 (s, 1H), 7.15-7.13 (d, 1H), 4.25 (bs, 1H), 3.73- 3.26 (m, 11H), 1.90-1.68 (m, 4H). LCMS: m/z = 502.7 (M + 1)⁺; HPLC: 97.77%. |
| 91 | 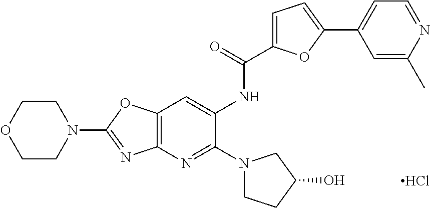 [SMILES: Cc1cc(-c2ccc(C(=O)Nc3cc4oc(N5CCOCC5)nc4nc3N3CC[C@@H](O)C3)o2)ccn1 ; Cl] | ¹HNMR (400 MHz, DMSO-d₆): δ 10.42 (s, 1H), 8.85-8.84 (d, 1H), 8.45 (s, 1H), 8.38-8.36 (d, 1H), 7.89-7.88 (d, 1H), 7.62 (s, 1H), 7.53-7.52 (d, 1H), 4.25 (bs, 1H), 3.73-3.24 (m, 13H), 1.90-1.68 (m, 2H). LCMS: m/z = 491.3 (M + 1)⁺; HPLC: 95.64%. |
| 92 | 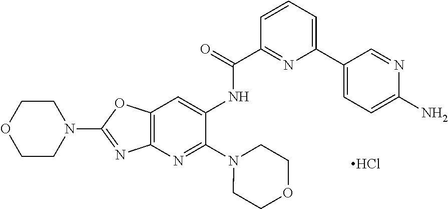 [SMILES: Nc1ccc(-c2cccc(C(=O)Nc3cc4oc(N5CCOCC5)nc4nc3N3CCOCC3)n2)cn1 ; Cl] | ¹HNMR (400 MHz, DMSO-d₆): δ 10.7 (s, 1H), 8.83-8.81 (m, 2H), 8.40 (bs, 2H), 8.26-8.16 (m, 4H), 7.19-7.16 (d, 1H), 3.74 (bs, 8H), 3.65-3.63 (m, 4H), 3.00-2.98 (m, 4H). LCMS: m/z = 503.4 (M + 1)⁺; HPLC: 98.08%. |
| 93 | 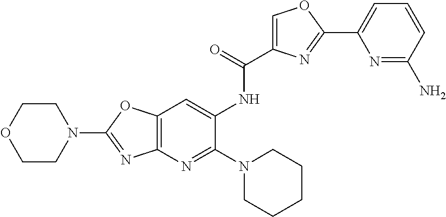 [SMILES: Nc1cccc(-c2nc(C(=O)Nc3cc4oc(N5CCOCC5)nc4nc3N3CCCCC3)co2)n1] | ¹HNMR (300 MHz, DMSO-d₆): δ 9.77 (s, 1H), 8.89 (s, 1H), 8.61 (s, 1H), 7.60 (t, 1H), 7.31-7.28 (d, 1H), 6.62-6.59 (d, 1H), 6.36 (s, 2H), 3.71-3.69 (m, 4H), 3.61- 3.60 (m, 4H), 2.92 (m, 4H), 1.79 (m, 4H), 1.60 (m, 2H). LCMS: m/z = 491.3 (M + 1)⁺; HPLC: 96.18% |
| 94 | 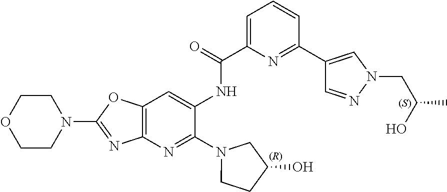 | ¹HNMR (400 MHz, CDCl₃): δ 10.60 (s, 1H), 8.75 (s, 1H), 8.34 (s, 1H), 8.09-8.07 (d, 1H), 8.01 (s, 1H), 7.89-7.85 (t, 1H), 7.64-7.62 (d, 1H), 4.46 (m, 1H), 4.31- 4.27 (m, 2H), 4.14-4.11 (m, 1H), 3.85- 3.73 (m, 8H), 3.45-3.42 (m, 1H), 3.29- 3.26 (m, 2H), 2.88 (m, 1H), 2.21-2.19 (m, 1H), 2.02-2.01 (m, 1H); 1.28-1.26 (d, 3H). LCMS: m/z = 535.0 (M + 1)⁺; HPLC: 98.88% |
| 95 | 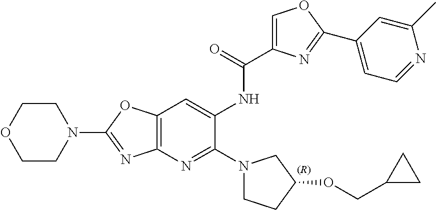 | ¹HNMR (400 MHz, CDCl₃): δ 9.19 (s, 1H), 8.67 (d, 1H), 8.40 (s, 1H), 8.37 (s, 1H), 7.82 (S, 1H), 7.75 (d, 1H), 4.27-4.24 (m, 1H), 3.83-3.62 (m, 10H), 3.47-3.29 (m, 4H), 2.68 (s, 3H), 2.20-2.08 (m, 2H), 1.04- 0.98 (m, 1H), 0.50-0.19 (m, 2H), 0.18-0.10 (m, 2H). LCMS: m/z = 546.2 (M + 1)⁺; HPLC: 97.57%. |
| 96 | 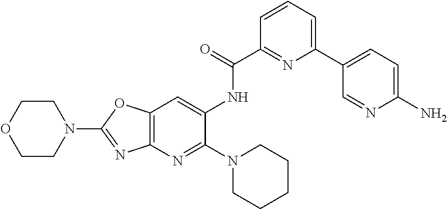 [SMILES: Nc1ccc(-c2cccc(C(=O)Nc3cc4oc(N5CCOCC5)nc4nc3N3CCCCC3)n2)cn1] | ¹HNMR (400 MHz, CDCl₃): δ 10.99 (s, 1H), 8.89 (s, 1H), 8.73 (s, 1H), 8.53 (dd, 1H), 8.23 (d, 1H), 7.99 (t, 1H), 7.87 (d, 1H), 6.73 (d, 1H), 5.30 (s, 2H), 3.84-3.82 (m, 4H), 3.76-3.74 (m, 4H), 3.06-3.03 (m, 4H), 1.85-1.72 (m, 4H), 1.57-1.55 (m, 2H). LCMS: m/z = 501.1 (M + 1)⁺; HPLC: 95.00% |
| 97 | 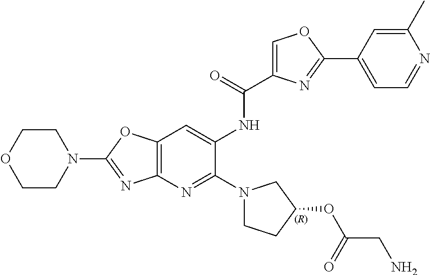 | ¹HNMR (400 MHz, CDCl₃): δ 9.18 (s, 1H), 8.70-8.69 (d, 1H), 8.40-8.35 (d, 2H), 7.79 (s, 1H), 7.73-7.72 (d, 1H), 5.50-5.40 (m, 1H), 3.90-3.73 (m, 10H), 3.70-3.60 (m, 2H), 3.50-3.40 (m, 5H), 2.68 (s, 3H), 2.40- 2.30 (m, 1H), 2.20-2.0 (m, 1H), LCMS: m/z = 549.2 (M + 1)⁺; HPLC: 94.64%. |

The below compounds were prepared by procedure similar to the one described in Example 19 with appropriate variations in reactants, quantities of reagents at suitable reaction conditions. The physicochemical characteristics of the compounds are summarized herein below table.

| Example | Structure | Analytical data |
|---|---|---|
| 98 | 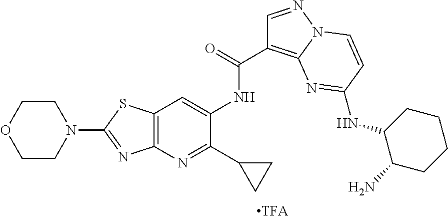 | ¹HNMR (400 MHz, CD₃OD): δ 8.57-8.55 (d, 1H), 8.37 (s, 1H), 8.21 (s, 1H), 6.66-6.64 (d, 1H), 4.74 (s, 1H), 4.20-4.10 (t, 1H), 3.80-3.60 (m, 8H), 3.05-3.00 (m, 1H), 2.90-2.80 (q, 1H), 2.40-2.20 (m, 1H), 2.05-1.60 (m, 4H), 1.20- 1.00 (m, 6H). LCMS: m/z = 534.3 (M + 1)⁺; HPLC: 98.81%. |
| 99 | 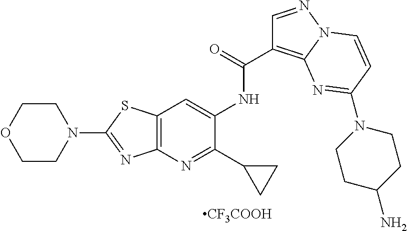 [SMILES: NC1CCN(c2ccn3ncc(C(=O)Nc4cc5sc(N6CCOCC6)nc5nc4C4CC4)c3n2)CC1 ; "·CF3COOH"] | ¹HNMR (400 MHz, DMSO-d₆): δ 9.59 (s, 1H), 8.94-8.92 (d, 1H), 8.48 (s, 1H), 8.38 (s, 1H), 7.91 (bs, 2H), 7.04-7.02 (d, 1H), 4.58 (bs, 1H), 3.80-3.60 (m, 8H), 3.50-3.30 (m, 4H), 2.39-2.33 (m, 1H), 2.06-2.04 (d, 2H), 1.58- 1.56 (q, 2H), 1.09-0.98 (m, 4H). LCMS: m/z = 520.3 (M + 1)⁺; HPLC: 97.48%. |
| 100 | 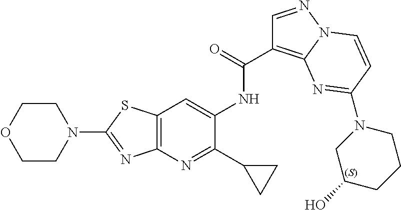 | ¹HNMR (400 MHz, CD₃OD): δ 9.62 (s, 1H), 8.83-8.1 (d, 1H), 8.49 (s, 1H), 8.33 (s, 1H), 6.97-6.95 (d, 1H), 4.10-3.60 (m, 12H), 2.39 (s, 1H), 1.92-1.83 (m, 3H), 1.59-1.55 (m, 3H), 1.06-1.03 (m, 4H). LCMS: m/z = 521.3 (M + 1)⁺; HPLC: 98.71%. |
| 101 | 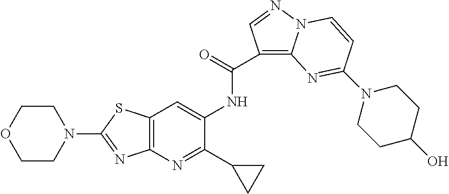 [SMILES: O=C(Nc1cc2sc(N3CCOCC3)nc2nc1C1CC1)c1cnn2ccc(N3CCC(O)CC3)nc12] | ¹HNMR (400 MHz, DMSO-d₆): δ 9.63 (s, 1H), 8.85-8.83 (d, 1H), 8.48 (s, 1H), 8.34 (s, 1H), 7.00-6.99 (d, 1H), 4.89-4.88 (d, 1H), 4.20-4.10 (m, 2H), 3.90-3.50 (m, 11H), 2.35- 2.32 (m, 2H), 1.86 (bs, 2H), 1.50-1.48 (q, 2H), 1.10-0.98 (m, 3H). LCMS: m/z = 521.3 (M + 1)⁺; HPLC: 95.32%. |
| 102 | 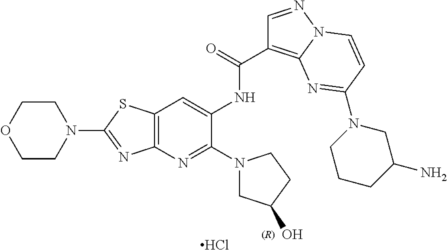 | ¹HNMR (400 MHz, DMSO-d₆): δ 9.185 (s, 1H), 8.89-8.87 (d, 1H), 8.30 (s, 1H), 8.20 (s, 1H), 8.13 (bs, 3H), 6.90-6.80 (d, 1H), 4.25 (brs, 2H), 4.10-4.0 (m, 2H), 3.74-3.73 (m, 4H), 3.67-3.57 (m, 4H), 3.60-3.58 (m, 2H), 3.25- 3.23 (d, 3H), 2.09-1.90 (m, 1H), 1.89-1.50 (m, 6H). LCMS: m/z = 565.2 (M + 1)⁺; HPLC: 95.06%. |
| 103 | 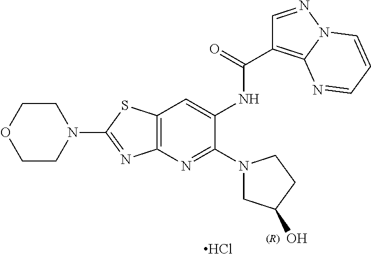 | ¹HNMR (400 MHz, DMSO-d₆): δ 9.55 (s, 1H), 9.38-9.36 (d, 1H), 8.87-8.85 (d, 1H), 8.70 (s, 1H), 8.24 (s, 1H), 7.34-7.31 (m, 1H), 4.83- 4.82 (d, 1H), 4.26 (s, 1H), 3.73-3.72 (m, 4H), 3.64-3.58 (m, 2H), 3.56-3.54 (m, 4H), 3.42- 3.38 (m, 1H), 3.10-2.90 (m, 1H), 1.90-1.89 (m, 1H), 1.75-1.74 (m, 1H). LCMS: m/z = 467.1 (M + 1)⁺; HPLC: 99.06%. |
| 104 | 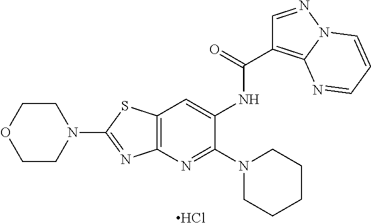 [SMILES: O=C(Nc1cc2sc(N3CCOCC3)nc2nc1N1CCCCC1)c1cnn2cccnc12 ; Cl] | ¹HNMR (400 MHz, DMSO-d₆): δ 10.34 (s, 1H), 9.38-9.36 (d, 1H), 9.06 (s, 1H), 9.90-8.89 (m, 1H), 8.70 (s, 1H), 7.34-7.31 (m, 1H), 3.71- 3.69 (bs, 4H), 3.52-3.48 (bs, 4H), 2.94-2.92 (bs, 4H), 1.73 (bs, 4H), 1.55 (bs, 2H. LCMS: m/z = 465.3 (M + 1)⁺; HPLC: 93.23%. |
| 105 | 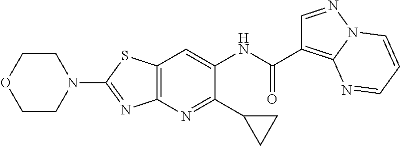 [SMILES: O=C(Nc1cc2sc(N3CCOCC3)nc2nc1C1CC1)c1cnn2cccnc12] | ¹HNMR (300 MHz, DMSO-d₆): δ 10.16 (s, 1H), 9.40-9.38 (dd, 1H), 8.91-8.90 (dd, 1H), 8.72-8.68 (d, 2H), 7.35-7.31 (m, 1H), 3.73- 3.70 (m, 4H), 3.58-3.55 (m, 4H), 2.32-2.26 (m, 1H), 1.05-1.00 (m, 4H). LCMS: m/z = 422.0 (M + 1)⁺; HPLC: 98.02% |
| 106 | 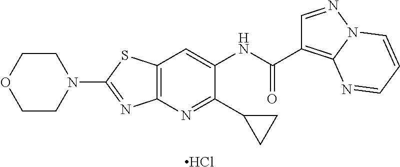 [SMILES: O=C(Nc1cc2sc(N3CCOCC3)nc2nc1C1CC1)c1cnn2cccnc12 ; Cl] | ¹HNMR (300 MHz, CD₃OD): δ 9.40 (s, 1H), 9.2 (d, 1H), 8.90-8.89 (d, 1H), 8.69 (s, 1H), 7.34-7.30 (m, 1H), 3.84 (s, 8H), 2.38-2.37 (m, 1H), 1.43-1.40 (m, 2H), 1.16-1.12 (m, 2H). LCMS: m/z = 421.95 (M + 1)⁺; HPLC: 98.02% |

### EXAMPLE 107

### N-(5-cyclopropyl-2-morpholinooxazolo[4,5-b]pyridin-6-yl)pyrazolo[1,5-a]pyrimidine-3-carboxamide hydrochloride

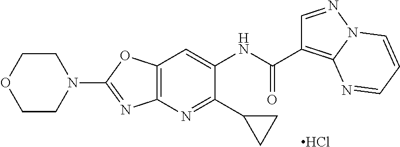 [SMILES: O=C(Nc1cc2oc(N3CCOCC3)nc2nc1C1CC1)c1cnn2cccnc12 ; Cl]

The title compound was prepared by procedure similar to the one described in Example 15 with appropriate variations in reactants, quantities of reagents at suitable reaction conditions.

¹HNMR (300 MHz, CDCl₃): δ 10.16 (s, 1H), 8.87-8.84 (dd, 1H), 8.79 (s, 1H), 8.71-8.69 (dd, 1H), 8.47 (s, 1H), 8.08 (s, 1H), 3.83-3.80 (m, 4H), 3.76-3.73 (m, 4H), 2.26 (m, 1H), 1.24-1.22 (m, 2H), 1.06-1.02 (m, 2H). LCMS: m/z=406.3 (M+1)⁺; HPLC: 98.54%

### IRAK-4 Biochemical Assay

Compounds were tested for their potential to inhibit IRAK-4 enzyme in a TR-FRET assay using recombinant IRAK-4 kinase from Millipore, USA. The assay buffer was 50 mM Tris-HCl pH 7.5, 20 mM MgCl₂, 1 mM EGTA, 2 mM DTT, 3 mM MnCl₂ and 0.01% Tween 20.5 ng of IRAK-4 kinase was used for the assay. After pre-incubation of enzyme with test compound for 30 minutes at room temperature, a substrate mixture containing 100 nM Biotin Histone H3 (Millipore, USA) and 20 μM ATP (Sigma, USA) was added and the reaction was incubated for 30 min. Post incubation, the reaction was stopped by the addition of stop mix containing 40 mM EDTA, 1 nM of Europium-Anti-Phospho-Histone H3 (Ser10) antibody (Perkin Elmer, USA) and 20 nM SureLight Allophycocyanin-Streptavidin (Perkin Elmer, USA). The fluorescence emission at 615 nm and 665 nm were measured at an excitation of 340 nm and the percent inhibition was estimated from the ratio of the fluorescence intensities [(F665/F615)×10000].

The compounds of the present invention were screened in the above mentioned assay and the percent inhibition data is summarized in Table 1. The IRAK-4 enzyme inhibitory rates at the concentrations of 0.1 μM and @ 1 μM are reported below. ‘NA’ indicates that the compounds were not tested at that concentration.

TABLE 1
Percent inhibition of IRAK-4 for the
compounds of the present invention

% inhibition

| Example | @0.1 μM | @ 1 μM |
|---|---|---|
| 2 | 98 | 99 |
| 3 | 97 | 98 |
| 4 | 99 | 99 |
| 5 | 98 | 99 |
| 6 | 100 | 100 |
| 7 | 99 | 99 |
| 8 | 97 | 98 |
| 9 | 101 | 99 |
| 10 | 100 | 100 |
| 11 | 99 | 98 |
| 12 | 98 | 98 |
| 13 | 98 | 98 |
| 14 | 99 | 100 |
| 15 | 96 | 100 |
| 16 | 94 | 99 |
| 17 | 94 | 98 |
| 18 | 95 | 98 |
| 19 | 98 | 99 |
| 20 | 97 | 98 |
| 21 | 97 | 98 |
| 22 | 97 | 98 |
| 23 | 86 | 97 |
| 24 | 97 | 97 |
| 25 | 55 | 96 |
| 26 | 87 | 92 |
| 27 | 95 | 97 |
| 28 | 90 | 93 |
| 29 | 98 | 99 |
| 30 | 98 | 99 |
| 31 | 90 | 98 |
| 32 | 93 | 95 |
| 33 | 95 | 97 |
| 34 | 97 | 99 |
| 35 | 92 | 95 |
| 36 | 94 | 99 |
| 37 | 75 | 95 |
| 38 | 98 | 99 |
| 39 | 83 | 98 |
| 40 | 96 | 99 |
| 41 | 92 | 98 |
| 42 | 94 | 98 |
| 43 | 50 | 92 |
| 44 | 90 | 96 |
| 45 | 95 | 97 |
| 46 | 75 | 94 |
| 47 | 18 | 58 |
| 48 | 96 | 100 |
| 49 | 79 | 96 |
| 50 | 67 | 95 |
| 51 | 98 | NA |
| 52 | 95 | 96 |
| 53 | 90 | 97 |
| 54 | 93 | 97 |
| 55 | 94 | 98 |
| 56 | 95 | 96 |
| 57 | 97 | 98 |
| 58 | 71 | 96 |
| 59 | 91 | 99 |
| 60 | 88 | 94 |
| 61 | 84 | 91 |
| 62 | 93 | 95 |
| 63 | 95 | 95 |
| 64 | 74 | 92 |
| 65 | 92 | 97 |
| 66 | 90 | 95 |
| 67 | 100 | 101 |
| 68 | 23 | 38 |
| 69 | 98 | 99 |
| 70 | 98 | 99 |
| 71 | 94 | 96 |
| 72 | 98 | 97 |
| 73 | 69 | 97 |
| 74 | 89 | 95 |
| 75 | 98 | 78 |
| 76 | 92 | 97 |
| 77 | 98 | 99 |
| 78 | 99 | 96 |
| 79 | 70 | 96 |
| 80 | 96 | 99 |
| 81 | 88 | 94 |
| 82 | 93 | 98 |
| 83 | 99 | 98 |
| 84 | 94 | 98 |
| 85 | 101 | 100 |
| 86 | 65 | 57 |
| 87 | 21 | 57 |
| 88 | 15 | NA |
| 89 | 26 | 73 |
| 90 | 87 | 97 |
| 91 | 45 | 92 |
| 92 | 78 | 96 |
| 93 | 87 | 93 |
| 94 | 12 | 60 |
| 95 | 80 | 97 |
| 96 | 97 | 96 |
| 97 | 87 | 99 |
| 98 | 94 | 97 |
| 99 | 89 | 96 |
| 100 | 96 | 99 |
| 101 | 96 | 98 |
| 102 | 21 | 56 |
| 103 | 92 | 97 |
| 104 | 95 | 97 |
| 105 | 96 | 97 |
| 106 | 93 | 97 |
| 107 | 57 | 92 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula (I) 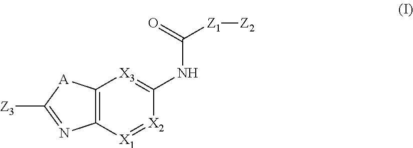
or a pharmaceutically acceptable salt or a stereoisomer thereof;
wherein
each X₁, X₂ and X₃ are independently CR² or N;
A is O, S, S(O) or S(O)₂;
Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;
Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};
Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};
each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;
each R′, R″ and R′″ is independently selected from hydrogen, alkyl, hydroxy, hydroxyalkyl, acyl and cycloalkyl;
each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl)alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;
each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or
Rₐ and R_{b} are taken together along with the atoms which they are attached to form a 3 to 8 membered optionally substituted ring; and
each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

2. A compound of formula (II) 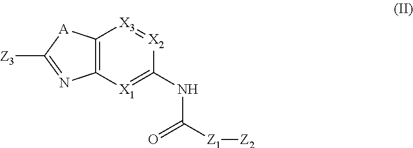
or a pharmaceutically acceptable salt or a stereoisomer thereof;
wherein
each X₁, X₂ and X₃ are independently CR² or N;
A is O, S, S(O) or S(O)₂;
Z₁ is optionally substituted heteroaryl, optionally substituted heterocycloalkyl, optionally substituted aryl, optionally substituted cycloalkyl, optionally substituted (heterocycloalkyl)alkyl-, optionally substituted aralkyl-, optionally substituted heteroaralkyl-, optionally substituted (cycloalkyl)alkyl-, optionally substituted aryloxy-, optionally substituted heteroaryloxy-, optionally substituted heterocycloalkyloxy-, optionally substituted cycloalkyloxy-, optionally substituted aryl-NR′—, optionally substituted heteroaryl-NR′—, optionally substituted heterocycloalkyl-NR′—, optionally substituted cycloalkyl-NR′—, optionally substituted aryl-S—, optionally substituted heteroaryl-S—, optionally substituted heterocycloalkyl-S—, optionally substituted cycloalkyl-S—, optionally substituted (cycloalkyl)alkyl-NR′—, optionally substituted aralkyl-NR′—, optionally substituted (heterocycloalkyl)alkyl-NR′—, optionally substituted heteroaralkyl-NR′—, optionally substituted (cycloalkyl)alkyl-S—, optionally substituted aralkyl-S—, optionally substituted (heterocycloalkyl)alkyl-S—, optionally substituted heteroaralkyl-S—, optionally substituted (cycloalkyl)alkyl-O—, optionally substituted aralkyl-O—, optionally substituted (heterocycloalkyl)alkyl-O—, optionally substituted heteroaralkyl-O—; e.g., wherein each optional substituent independently represents an occurance of Rₓ;
Z₂ is absent or optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)alkyl-NR″—, optionally substituted aralkyl-NR″—, optionally substituted (heterocycloalkyl)alkyl-NR″—, optionally substituted heteroaralkyl-NR″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{y};
Z₃ is optionally substituted cycloalkyl, optionally substituted aryl, optionally substituted heterocycloalkyl, optionally substituted heteroaryl, optionally substituted aryloxy-, optionally substituted heteroaryloxy-, optionally substituted cycloalkyloxy-, optionally substituted heterocycloalkyloxy-, optionally substituted (cycloalkyl)alkyl-, optionally substituted aralkyl-, optionally substituted (heterocycloalkyl)alkyl-, optionally substituted heteroaralkyl-, optionally substituted (cycloalkyl)-NR′″—, optionally substituted aryl-NR′″—, optionally substituted heteroaryl-NR′″—, optionally substituted heterocycloalkyl-NR′″—, optionally substituted aryl-S—, optionally substituted heteroaryl-S—, optionally substituted cycloalkyl-S—, optionally substituted heterocycloalkyl-S—, optionally substituted (cycloalkyl)alkyl-NR′″—, optionally substituted aralkyl-NR′″—, optionally substituted (heterocycloalkyl)alkyl-NR′″—, optionally substituted heteroaralkyl-NR′″—, optionally substituted (cycloalkyl)alkyl-O—, optionally substituted aralkyl-O—, optionally substituted (heterocycloalkyl)alkyl-O—, optionally substituted heteroaralkyl-O—, optionally substituted (cycloalkyl)alkyl-S—, optionally substituted aralkyl-S—, optionally substituted (heterocycloalkyl)alkyl-S— or optionally substituted heteroaralkyl-S—; e.g., wherein each optional substituent independently represents an occurance of R_{z};
each R² is independently selected from hydrogen, alkyl, haloalkyl, halo, cyano, optionally substituted alkoxy, optionally substituted cycloalkyl, optionally substituted (cycloalkyl)alkyl-, optionally substituted cycloalkyloxy-, optionally substituted aryl, optionally substituted aralkyl-, optionally substituted heterocycloalkyl, optionally substituted heteroaryl, optionally substituted (heterocycloalkyl)alkyl-, optionally substituted heteroaralkyl-, —NRₐR_{b}, —O—R₃ and —S—R₃; e.g., wherein each optional substituent independently represents alkyl, alkoxy, halo, haloalkyl, hydroxy, hydroxyalkyl, —SH, —S(alkyl), cyano, amido, amino, carboxylate, glycinate, alaninate, oxo, aryl, cycloalkyl, heterocycloalkyl or heteroaryl;
each R′, R″ and R′″ is independently selected from hydrogen, alkyl, hydroxy, hydroxyalkyl, acyl and cycloalkyl;
each Rₓ, R_{y} and R_{z} is independently selected from alkyl, alkenyl, alkynyl, halo, hydroxy, haloalkyl, hydroxyalkyl, aminoalkyl, alkoxy, —SH, —S(alkyl), cyano, amido, carboxylic acid, carboxylate, ester, thioester, alkoxycarbonyl, —C(O)NH(alkyl), oxo, cycloalkyl, cycloalkyloxy, (cycloalkyl)alkyl-, aryl, aralkyl-, heterocycloalkyl, heteroaryl, (heterocycloalkyl)alkyl-, heteroaralkyl-, —NRₐR_{b}, —O—R₄ or —S—R₄; optionally wherein the cycloalkyl, aryl, heterocycloalkyl, and heteroaryl are further substituted by one or more substituents selected from halo, haloalkyl, amino, hydroxy, alkyl, cyano, nitro, alkenyl, aminoalkyl, hydroxyalkyl and haloalkoxy;
each Rₐ and R_{b} is independently selected from hydrogen, alkyl, aminoalkyl, acyl, aminoacyl, halo, haloalkyl, hydroxy, haloalkoxy, hydroxyalkyl, nitro, cyano, cycloalkyl, heterocycloalkyl, aryl, heteroaryl, (cycloalkyl)alkyl-, (heterocycloalkyl)alkyl-, aralkyl-, and (heteroaryl)alkyl-; optionally wherein the cycloalkyl, heterocycloalkyl, aryl and heteroaryl are further substituted by one or more substituents selected from alkyl, halo, alkenyl, cyano, hydroxy, hydroxyalkyl, alkoxy, amino and nitro; or
Rₐ and R_{b} are taken together along with the atoms which they are attached to form a 3 to 8 membered optionally substituted ring; and
each R₃ and R₄ is independently selected from hydrogen, alkyl, aminoacyl, phosphate, phosphonate, alkylphosphate, alkoxycarbonyl, cycloalkyl, (cycloalkyl)alkyl-, aryl, heteroaryl, heterocycloalkyl, aralkyl-, heteroaralkyl and (heterocycloalkyl)alkyl-.

3. The compound of formula (I) according to claim 1, wherein the group 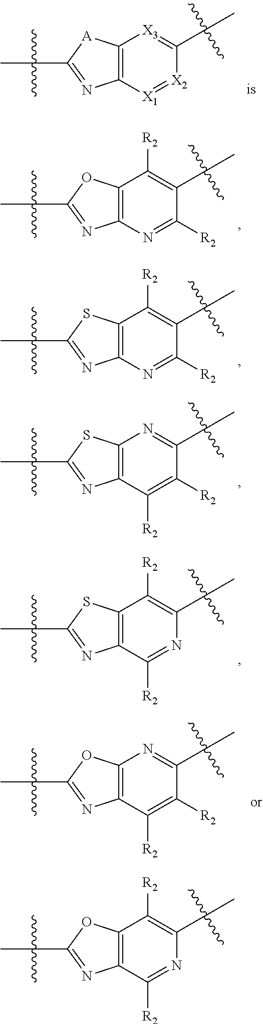
wherein [symbol] is a point of attachment and R₂ is as defined in claim 1.

4. The compound of formula (I) according to claim 2, wherein the group 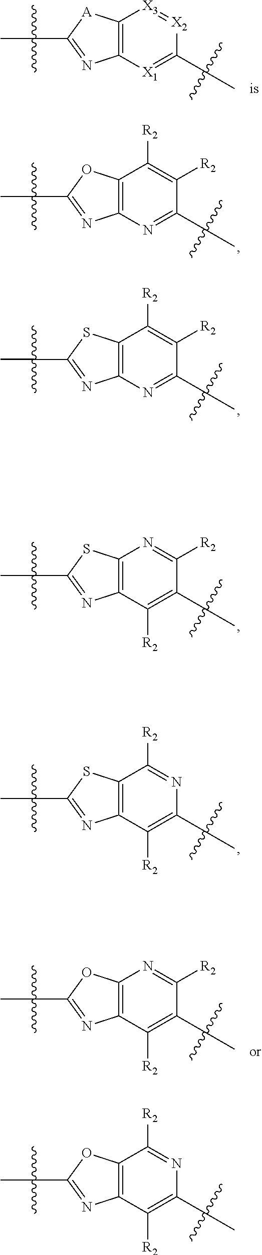
wherein [symbol] is a point of attachment and R₂ is as defined in claim 2.

5. The compound of any one of claims 1-4, wherein Z₁ is optionally substituted heteroaryl, optionally substituted heterocycloalkyl, optionally substituted aryl or optionally substituted cycloalkyl; wherein each optional substituent independently represents an occurrence of Rₓ, and Rₓ is as defined in claim 1 or 2.

6. The compound of claim 5, wherein Z₁ is optionally substituted monocyclic heterocycloalkyl or optionally substituted monocyclic heteroaryl, wherein each optional substituent independently represents an occurrence of Rₓ, and Rₓ is as defined in claim 1 or 2.

7. The compound of claim 5, wherein Z₁ is optionally substituted bicyclic heterocycloalkyl or optionally substituted bicyclic heteroaryl, wherein each optional substituent independently represents an occurrence of Rₓ and Rₓ is as defined in claim 1 or 2.

8. The compound of any one of claims 1 to 7, wherein Z₁ is selected from phenyl, naphthyl, furanyl, thienyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl, pyridazinyl, benzimidazolyl, benzoxadiazolyl, benzoxathiadiazolyl, cinnolinyl, furopyridinyl, naphthyridinyl, quinolinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carboline, indolizinyl, benzoisothiazolyl, benzoxazolyl, pyrrolopyridyl, purinyl, benzotriazolyl, benzotriadiazolyl, carbazolyl, dibenzothienyl, acridinyl, pyrazolopyrimidyl, azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl or azabicyclo[3.2.1]octanyl; each of which is optionally substituted, and each optional substituent independently represents an occurrence of Rₓ, and Rₓ is as defined in claim 1 or 2.

9. The compound any one of claims 1 to 8, wherein Z₂ is optionally substituted heterocycloalkyl, optionally substituted heteroaryl or is absent; wherein each optional substituent independently represents an occurrence of R_{y}; and R_{y} is as defined in claim 1 or 2.

10. The compound of claim 9, wherein Z₂ is absent.

11. The compound of any one of claims 1 to 10, wherein Z₃ is optionally substituted cycloalkyl, optionally substituted aryl, optionally substituted heterocycloalkyl, and optionally substituted heteroaryl; wherein each optional substituent independently represents an occurrence of R_{z}; and R_{z} is as defined in claim 1 or 2.

12. The compound of claim 11, wherein Z₃ is optionally substituted heterocycloalkyl or optionally substituted heteroaryl; wherein each optional substituent independently represents an occurrence of R_{z}; and R_{z} is as defined in claim 1 or 2.

13. The compound of any one of claims 1 to 8, wherein Rₓ is alkyl, halo, hydroxy, haloalkyl, hydroxyalkyl, aminoalkyl, alkoxy, amido, carboxylic acid, carboxylate, oxo, cycloalkyl, aryl, —NRₐR_{b} or —O—R₄; optionally wherein the cycloalkyl and aryl are further substituted by one or more substituents selected from halo, haloalkyl, amino, hydroxy, alkyl, cyano, aminoalkyl, hydroxyalkyl and haloalkoxy; wherein Rₐ, R_{b}, and R₄ are as defined in claim 1 or 2.

14. The compound of any one of claim 1-4 or 9, wherein R_{y} is alkyl, halo, hydroxy, haloalkyl, hydroxyalkyl, aminoalkyl, alkoxy, cyano, amido, carboxylic acid, carboxylate, ester, alkoxycarbonyl, oxo, cycloalkyl, aryl, aralkyl, heterocycloalkyl, heteroaryl, (heterocycloalkyl)alkyl-, heteroaralkyl, —NRₐR_{b}, —O—R₄ or; optionally wherein the cycloalkyl, aryl, heterocycloalkyl, heteroaryl are further substituted by one or more substituents selected from halo, haloalkyl, amino, hydroxy, alkyl, cyano, nitro, alkenyl, aminoalkyl, hydroxyalkyl and haloalkoxy; wherein Rₐ, R_{b}, and R₄ are as defined in claim 1 or 2.

15. The compound of claim 1, wherein at least one occurrence of R² is haloalkyl, optionally substituted alkyl, optionally substituted cycloalkyl, optionally substituted aryl, optionally substituted heterocycloalkyl, optionally substituted heteroaryl, —NRₐR_{b}, —O—R₃ or —S—R₃; wherein each optional substituent is independently selected from alkyl, alkoxy, halo, haloalkyl, hydroxy, hydroxyalkyl, amido, amino, carboxylate, oxo or cycloalkyl; wherein Rₐ, R_{b}, and R₃ are as defined in claim 1 or 2.

16. The compound of any preceding claim, wherein X₁, X₂ and X₃ are each independently CR² or N; provided that at least one of X₁, X₂ and X₃ is N.

17. The compound of claim 1, having the structure of compound of formula (IA): 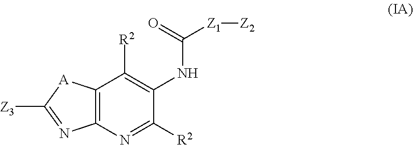
or a pharmaceutically acceptable salt or a stereoisomer thereof;
wherein Z₁, Z₂, Z₃, A and R² are as defined in claim 1.

18. The compound of claim 2, having the structure of formula (IIA): 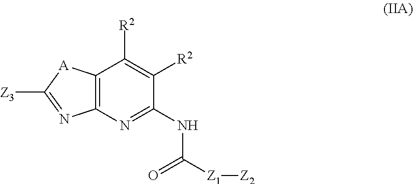
or a pharmaceutically acceptable salt or a stereoisomer thereof;
wherein Z₁, Z₂, Z₃, A and R² are as defined in claim 2.

19. A compound selected from

| Example | IUPAC name |
|---|---|
| 1 | N-(2-(4-methylpiperazin-1-yl)-5-(piperidin-1-yl)thiazolo[4,5-b]pyridin-6-yl)-2- |
| | (2-methylpyridin-4-yl)oxazole-4-carboxamide hydrochloride; |
| 2 | N-(5-(4-hydroxy-4-(hydroxymethyl)piperidin-1-yl)-2-morpholinothiazolo[4,5- |
| | b]pyridin-6-yl)-5-(2-methylpyridin-4-yl)furan-2-carboxamide hydrochloride; |
| 3 | 2-(1-methyl-6-oxo-1,6-dihydropyridin-3-yl)-N-(2-morpholino-5-(piperidin-1- |
| | yl)thiazolo[4,5-b]pyridin-6-yl)oxazole-4-carboxamide; |
| 4 | N-(5-(4-hydroxy-4-(hydroxymethyl)piperidin-1-yl)-2-morpholinothiazolo[4,5- |
| | b]pyridin-6-yl)-2-(2-methylpyridin-4-yl)oxazole-5-carboxamide hydrochloride; |
| 5 | N-(5-(3-hydroxy-3-(hydroxymethyl)piperidin-1-yl)-2-morpholinothiazolo[4,5- |
| | b]pyridin-6-yl)-5-(2-methylpyridin-4-yl)furan-2-carboxamide hydrochloride; |
| 6 | 2-(2-methylpyridin-4-yl)-N-(2-morpholino-5-(((1r,4r)-4- |
| | morpholinocyclohexyl)oxy)thiazolo[4,5-b]pyridin-6-yl)oxazole-4-carboxamide |
| | hydrochloride; |
| 7 | 2-(2-methylpyridin-4-yl)-N-(2-(piperazin-1-yl)-5-(piperidin-1-yl)thiazolo[4,5- |
| | b]pyridin-6-yl)oxazole-4-carboxamide hydrochloride; |
| 8 | (S)-N-(5-(4-hydroxy-4-(hydroxymethyl)piperidin-1-yl)-2- |
| | morpholinothiazolo[4,5-b]pyridin-6-yl)-6-(3-hydroxypyrrolidin-1- |
| | yl)picolinamide hydrochloride; |
| 9 | N-(5-(3-hydroxy-3-(hydroxymethyl)pyrrolidin-1-yl)-2-morpholinothiazolo[4,5- |
| | b]pyridin-6-yl)-2-(2-methylpyridin-4-yl)oxazole-4-carboxamide hydrochloride; |
| 10 | N-(2-(4-(2-hydroxyacetyl)piperazin-1-yl)-5-(piperidin-1-yl)thiazolo[4,5- |
| | b]pyridin-6-yl)-2-(2-methylpyridin-4-yl)oxazole-4-carboxamide hydrochloride; |
| 11 | 2-(6-aminopyridin-2-yl)-N-(2-morpholino-5-(piperidin-1-yl)thiazolo[4,5- |
| | b]pyridin-6-yl)oxazole-4-carboxamide hydrochloride; |
| 12 | 2-(2-amino-5-chloropyridin-4-yl)-N-(2-morpholino-5-(piperidin-1- |
| | yl)thiazolo[4,5-b]pyridin-6-yl)oxazole-4-carboxamide; |
| 13 | 2-(5-fluoro-1-methyl-6-oxo-1,6-dihydropyridin-3-yl)-N-(2-morpholino-5- |
| | (piperidin-1-yl)thiazolo[4,5-b]pyridin-6-yl)oxazole-4-carboxamide; |
| 14 | N-(5-(5-methylpyridin-2-yl)-2-morpholinooxazolo[4,5-b]pyridin-6-yl)-2-(2- |
| | methylpyridin-4-yl)oxazole-4-carboxamide; |
| 15 | N-(5-(3-hydroxy-3-(hydroxymethyl)piperidin-1-yl)-2-morpholinooxazolo[4,5- |
| | b]pyridin-6-yl)-2-(2-methylpyridin-4-yl)oxazole-4-carboxamide; |
| 16 | 2-(2-methylpyridin-4-yl)-N-(2-morpholino-5-(((1r,4r)-4- |
| | morpholinocyclohexyl)oxy)oxazolo[4,5-b]pyridin-6-yl)oxazole-4-carboxamide |
| | hydrochloride; |
| 17 | (S)-N-(5-(3-fluoropyrrolidin-1-yl)-2-morpholinooxazolo[4,5-b]pyridin-6-yl)-2- |
| | (2-methylpyridin-4-yl)oxazole-4-carboxamide; |
| 18 | (R)-N-(5-(3-fluoropyrrolidin-1-yl)-2-morpholinooxazolo[4,5-b]pyridin-6-yl)-2- |
| | (2-methylpyridin-4-yl)oxazole-4-carboxamide hydrochloride; |
| 19 | N-(2-morpholino-5-(piperidin-1-yl)thiazolo[4,5-b]pyridin-6-yl)pyrazolo[1,5- |
| | a]pyrimidine-3-carboxamide; |
| 20 | N-(5-(5-methylpyridin-2-yl)-2-morpholinothiazolo[4,5-b]pyridin-6- |
| | yl)pyrazolo[1,5-a]pyrimidine-3-carboxamide; |
| 21 | 5-(3-aminopiperidin-1-yl)-N-(5-cyclopropyl-2-morpholinothiazolo[4,5-b]pyridin- |
| | 6-yl)pyrazolo[1,5-a]pyrimidine-3-carboxamide; |
| 22 | 5-((2-aminoethyl)amino)-N-(5-cyclopropyl-2-morpholinothiazolo[4,5-b]pyridin- |
| | 6-yl)pyrazolo[1,5-a]pyrimidine-3-carboxamide; |
| 23 | 5-(3-aminopyrrolidin-1-yl)-N-(5-cyclopropyl-2-morpholinothiazolo[4,5- |
| | b]pyridin-6-yl)pyrazolo[1,5-a]pyrimidine-3-carboxamide hydrochloride; |
| 24 | N-(2-morpholino-5-(piperidin-1-yl)oxazolo[4,5-b]pyridin-6-yl)pyrazolo[1,5- |
| | a]pyrimidine-3-carboxamide; |
| 25 | (R)-N-(5-(3-hydroxypyrrolidin-1-yl)-2-morpholinooxazolo[4,5-b]pyridin-6- |
| | yl)pyrazolo[1,5-a]pyrimidine-3-carboxamide; |
| 26 | (S)-6-(3-aminopyrrolidin-1-yl)-N-(2-morpholino-5-(piperidin-1-yl)thiazolo[4,5- |
| | b]pyridin-6-yl)picolinamide; |
| 27 | (S)-6-(3-(2-aminoacetamido)pyrrolidin-1-yl)-N-(2-morpholino-5-(piperidin-1- |
| | yl)thiazolo[4,5-b]pyridin-6-yl)picolinamide; |
| 28 | N-(5-((R)-3-hydroxypiperidin-1-yl)-2-morpholinothiazolo[4,5-b]pyridin-6-yl)-6- |
| | ((S)-3-hydroxypyrrolidin-1-yl)picolinamide; |
| 29 | (S)-6-(3-hydroxypiperidin-1-yl)-N-(2-morpholino-5-(piperidin-1- |
| | yl)thiazolo[4,5-b]pyridin-6-yl)picolinamide; |
| 30 | (R)-6-(3-hydroxypiperidin-1-yl)-N-(2-morpholino-5-(piperidin-1- |
| | yl)thiazolo[4,5-b]pyridin-6-yl)picolinamide; |
| 31 | 6-(4-methoxypiperidin-1-yl)-N-(2-morpholino-5-(piperidin-1-yl)thiazolo[4,5- |
| | b]pyridin-6-yl)picolinamide; |
| 32 | 6-(4-(hydroxymethyl)piperidin-1-yl)-N-(2-morpholino-5-(piperidin-1- |
| | yl)thiazolo[4,5-b]pyridin-6-yl)picolinamide; |
| 33 | (R)-6-(2-(hydroxymethyl)morpholino)-N-(2-morpholino-5-(piperidin-1- |
| | yl)thiazolo[4,5-b]pyridin-6-yl)picolinamide; |
| 34 | (R)-6-(3-hydroxypyrrolidin-1-yl)-N-(2-morpholino-5-(piperidin-1- |
| | yl)thiazolo[4,5-b]pyridin-6-yl)picolinamide; |
| 35 | 6-(4-(2-hydroxyethyl)piperidin-1-yl)-N-(2-morpholino-5-(piperidin-1- |
| | yl)thiazolo[4,5-b]pyridin-6-yl)picolinamide; |
| 36 | (S)-1-(6-((2-morpholino-5-(piperidin-1-yl)thiazolo[4,5-b]pyridin-6- |
| | yl)carbamoyl)pyridin-2-yl)pyrrolidin-3-ylglycinate 2,2,2-trifluoroacetate; |
| 37 | N-(2-morpholino-5-(piperidin-1-yl)thiazolo[4,5-b]pyridin-6-yl)-6-(((tetrahydro- |
| | 2H-pyran-4-yl)methyl)amino)picolinamide; |
| 38 | (R)-6-(4-hydroxypiperidin-1-yl)-N-(5-(3-hydroxypiperidin-1-yl)-2- |
| | morpholinothiazolo[4,5-b]pyridin-6-yl)picolinamide; |
| 39 | (R)-6-(azepan-1-yl)-N-(5-(3-hydroxypiperidin-1-yl)-2-morpholinothiazolo[4,5- |
| | b]pyridin-6-yl)picolinamide; |
| 40 | (R)-N-(5-(3-hydroxypiperidin-1-yl)-2-morpholinothiazolo[4,5-b]pyridin-6-yl)-6- |
| | (piperidin-1-yl)picolinamide; |
| 41 | (R)-N-(5-(3-hydroxypiperidin-1-yl)-2-morpholinothiazolo[4,5-b]pyridin-6-yl)-2′- |
| | methyl-[2,4′-bipyridine]-6-carboxamide; |
| 42 | (S)-6-(3-hydroxypyrrolidin-1-yl)-N-(2-morpholino-5-(piperidin-1- |
| | yl)thiazolo[4,5-b]pyridin-6-yl)picolinamide hydrochloride; |
| 43 | 6-(3-hydroxypyrrolidin-1-yl)-N-(5-methyl-2-morpholinothiazolo[4,5-b]pyridin- |
| | 6-yl)picolinamide; |
| 44 | 6-(3-hydroxypyrrolidin-1-yl)-N-(2-morpholino-5-(trifluoromethyl)thiazolo[4,5- |
| | b]pyridin-6-yl)picolinamide; |
| 45 | 6-(2-oxa-5-azabicyclo[2.2.1]heptan-5-yl)-N-(2-morpholino-5-(piperidin-1- |
| | yl)thiazolo[4,5-b]pyridin-6-yl)picolinamide; |
| 46 | 6-((4-(hydroxymethyl)cyclohexyl)amino)-N-(2-morpholino-5-(piperidin-1- |
| | yl)thiazolo[4,5-b]pyridin-6-yl)picolinamide; |
| 47 | (S)-diethyl (1-(6-((2-morpholino-5-(piperidin-1-yl)thiazolo[4,5-b]pyridin-6- |
| | yl)carbamoyl)pyridin-2-yl)pyrrolidin-3-yl)phosphate; |
| 48 | methyl 1-(6-((2-morpholino-5-(piperidin-1-yl)thiazolo[4,5-b]pyridin-6- |
| | yl)carbamoyl)pyridin-2-yl)-2-oxoimidazolidine-4-carboxylate; |
| 49 | 1-(6-((2-morpholino-5-(piperidin-1-yl)thiazolo[4,5-b]pyridin-6- |
| | yl)carbamoyl)pyridin-2-yl)-2-oxoimidazolidine-4-carboxylic acid; |
| 50 | N-(5-cyclopropyl-2-morpholinothiazolo[4,5-b]pyridin-6-yl)-6-(3- |
| | hydroxypyrrolidin-1-yl)pyrazine-2-carboxamide; |
| 51 | 6′-amino-N-(5-cyclopropyl-2-morpholinothiazolo[4,5-b]pyridin-6-yl)-[2,3′- |
| | bipyridine]-6-carboxamide; |
| 52 | 2′-amino-N-(5-cyclopropyl-2-morpholinothiazolo[4,5-b]pyridin-6-yl)-[2,4′- |
| | bipyridine]-6-carboxamide; |
| 53 | 2-(3-hydroxypyrrolidin-1-yl)-N-(2-morpholino-5-(piperidin-1-yl)thiazolo[4,5- |
| | b]pyridin-6-yl)pyrimidine-4-carboxamide; |
| 54 | 6-(3-hydroxypyrrolidin-1-yl)-N-(2-morpholino-5-(piperidin-1-yl)thiazolo[4,5- |
| | b]pyridin-6-yl)pyrazine-2-carboxamide hydrochloride; |
| 55 | 6-(3-hydroxypiperidin-1-yl)-N-(2-morpholino-5-(piperidin-1-yl)thiazolo[4,5- |
| | b]pyridin-6-yl)picolinamide; |
| 56 | 6-(4-hydroxypiperidin-1-yl)-N-(2-morpholino-5-(piperidin-1-yl)thiazolo[4,5- |
| | b]pyridin-6-yl)picolinamide; |
| 57 | (S)-1-(6-((2-morpholino-5-(piperidin-1-yl)thiazolo[4,5-b]pyridin-6- |
| | yl)carbamoyl)pyridin-2-yl)pyrrolidin-3-ylalaninate; |
| 58 | (S)-ethyl (1-(6-((2-morpholino-5-(piperidin-1-yl)thiazolo[4,5-b]pyridin-6- |
| | yl)carbamoyl)pyridin-2-yl)pyrrolidin-3-yl)carbonate; |
| 59 | (R)-2-(3-hydroxypyrrolidin-1-yl)-N-(2-morpholino-5-(piperidin-1- |
| | yl)thiazolo[4,5-b]pyridin-6-yl)pyrimidine-4-carboxamide; |
| 60 | (S)-2-(3-hydroxypyrrolidin-1-yl)-N-(2-morpholino-5-(piperidin-1- |
| | yl)thiazolo[4,5-b]pyridin-6-yl)pyrimidine-4-carboxamide; |
| 61 | N-(2-morpholino-5-(piperidin-1-yl)thiazolo[4,5-b]pyridin-6-yl)-6-(3- |
| | oxopiperazin-1-yl)picolinamide; |
| 62 | (R)-6-(3-hydroxypyrrolidin-1-yl)-N-(2-morpholino-5-(piperidin-1- |
| | yl)thiazolo[4,5-b]pyridin-6-yl)pyrazine-2-carboxamide; |
| 63 | (S)-6-(3-hydroxypyrrolidin-1-yl)-N-(2-morpholino-5-(piperidin-1- |
| | yl)thiazolo[4,5-b]pyridin-6-yl)pyrazine-2-carboxamide; |
| 64 | (S)-1-(4-((2-morpholino-5-(piperidin-1-yl)thiazolo[4,5-b]pyridin-6- |
| | yl)carbamoyl)pyrimidin-2-yl)pyrrolidin-3-yl glycinate; |
| 65 | (S)-1-(6-((2-morpholino-5-(piperidin-1-yl)thiazolo[4,5-b]pyridin-6- |
| | yl)carbamoyl)pyrazin-2-yl)pyrrolidin-3-yl glycinate; |
| 66 | 1-(6-((2-morpholino-5-(piperidin-1-yl)thiazolo[4,5-b]pyridin-6- |
| | yl)carbamoyl)pyridin-2-yl)piperidin-4-yl glycinate; |
| 67 | 6-(4-(hydroxymethyl)-1H-imidazol-1-yl)-N-(2-morpholino-5-(piperidin-1- |
| | yl)thiazolo[4,5-b]pyridin-6-yl)picolinamide; |
| 68 | (S)-4-(3-hydroxypyrrolidin-1-yl)-N-(2-morpholino-5-(piperidin-1- |
| | yl)thiazolo[4,5-b]pyridin-6-yl)picolinamide; |
| 69 | 6-(4-(hydroxymethyl)-1H-pyrazol-1-yl)-N-(2-morpholino-5-(piperidin-1- |
| | yl)thiazolo[4,5-b]pyridin-6-yl)picolinamide; |
| 70 | 6-(4-(aminomethyl)-1H-imidazol-1-yl)-N-(2-morpholino-5-(piperidin-1- |
| | yl)thiazolo[4,5-b]pyridin-6-yl)picolinamide 2,2,2-trifluoroacetate; |
| 71 | sodium 1-(6-((2-morpholino-5-(piperidin-1-yl)thiazolo[4,5-b]pyridin-6- |
| | yl)carbamoyl)pyridin-2-yl)piperidine-4-carboxylate; |
| 72 | (R)-N-(5-(3-hydroxypyrrolidin-1-yl)-2-morpholinothiazolo[4,5-b]pyridin-6-yl)- |
| | 4-(2-methylpyridin-4-yl)-1H-imidazole-2-carboxamide 2,2,2-trifluoroacetate; |
| 73 | (R)-N-(5-(3-hydroxypyrrolidin-1-yl)-2-morpholinothiazolo[4,5-b]pyridin-6-yl)- |
| | 4-(2-methylpyridin-4-yl)-1H-pyrrole-2-carboxamide; |
| 74 | 2-(5-methoxypyridin-2-yl)-N-(2-morpholino-5-(piperidin-1-yl)thiazolo[4,5- |
| | b]pyridin-6-yl)oxazole-4-carboxamide; |
| 75 | 6′-amino-N-(2-morpholino-5-(piperidin-1-yl)thiazolo[4,5-b]pyridin-6-yl)-[2,3′- |
| | bipyridine]-6-carboxamide; |
| 76 | N-(2,5-dimorpholinothiazolo[4,5-b]pyridin-6-yl)-6-(3-hydroxypyrrolidin-1- |
| | yl)picolinamide; |
| 77 | 6-(3-(hydroxymethyl)pyrrolidin-1-yl)-N-(2-morpholino-5-(piperidin-1- |
| | yl)thiazolo[4,5-b]pyridin-6-yl)picolinamide; |
| 78 | 6-(3-(hydroxymethyl)piperidin-1-yl)-N-(2-morpholino-5-(piperidin-1- |
| | yl)thiazolo[4,5-b]pyridin-6-yl)picolinamide; |
| 79 | 1-(6-((2,5-dimorpholinothiazolo[4,5-b]pyridin-6-yl)carbamoyl)pyridin-2- |
| | yl)pyrrolidin-3-yl glycinate; |
| 80 | 1-(6-((2-morpholino-5-(piperidin-1-yl)thiazolo[4,5-b]pyridin-6- |
| | yl)carbamoyl)pyridin-2-yl)piperidine-4-carboxylic acid; |
| 81 | 6-(3-hydroxy-8-azabicyclo[3.2.1]octan-8-yl)-N-(2-morpholino-5-(piperidin-1- |
| | yl)thiazolo[4,5-b]pyridin-6-yl)picolinamide; |
| 82 | 6-(4-carbamoylpiperidin-1-yl)-N-(2-morpholino-5-(piperidin-1-yl)thiazolo[4,5- |
| | b]pyridin-6-yl)picolinamide; |
| 83 | 2′-amino-N-(2-morpholino-5-(piperidin-1-yl)thiazolo[4,5-b]pyridin-6-yl)-[2,4′- |
| | bipyridine]-6-carboxamide; |
| 84 | (S)-6-(3-hydroxypyrrolidin-1-yl)-5-methyl-N-(2-morpholino-5-(piperidin-1- |
| | yl)thiazolo[4,5-b]pyridin-6-yl)picolinamide; |
| 85 | 6′-amino-3-methyl-N-(2-morpholino-5-(piperidin-1-yl)thiazolo[4,5-b]pyridin-6- |
| | yl)-[2,3′-bipyridine]-6-carboxamide; |
| 86 | N-(5-(4,4-dimethyl-2-oxopyrrolidin-1-yl)-2-morpholinooxazolo[4,5-b]pyridin-6- |
| | yl)-2-(2-methylpyridin-4-yl)oxazole-4-carboxamide; |
| 87 | (R)-2-(6-aminopyridin-2-yl)-N-(5-(3-hydroxypyrrolidin-1-yl)-2- |
| | morpholinooxazolo[4,5-b]pyridin-6-yl)oxazole-4-carboxamide hydrochloride; |
| 88 | (R)-2-(2-amino-5-chloropyridin-4-yl)-N-(5-(3-hydroxypyrrolidin-1-yl)-2- |
| | morpholinooxazolo[4,5-b]pyridin-6-yl)oxazole-4-carboxamide; |
| 89 | 2-(2-methylpyridin-4-yl)-N-(2-morpholino-5-(2-oxopyrrolidin-1-yl)oxazolo[4,5- |
| | b]pyridin-6-yl)oxazole-4-carboxamide hydrochloride; |
| 90 | (R)-6′-amino-N-(5-(3-hydroxypyrrolidin-1-yl)-2-morpholinooxazolo[4,5- |
| | b]pyridin-6-yl)-[2,3′-bipyridine]-6-carboxamide hydrochloride; |
| 91 | (R)-N-(5-(3-hydroxypyrrolidin-1-yl)-2-morpholinooxazolo[4,5-b]pyridin-6-yl)-5- |
| | (2-methylpyridin-4-yl)furan-2-carboxamide hydrochloride; |
| 92 | 6′-amino-N-(2,5-dimorpholinooxazolo[4,5-b]pyridin-6-yl)-[2,3′-bipyridine]-6- |
| | carboxamide hydrochloride; |
| 93 | 2-(6-aminopyridin-2-yl)-N-(2-morpholino-5-(piperidin-1-yl)oxazolo[4,5- |
| | b]pyridin-6-yl)oxazole-4-carboxamide; |
| 94 | 6-(1-((S)-2-hydroxypropyl)-1H-pyrazol-4-yl)-N-(5-((R)-3-hydroxypyrrolidin-1- |
| | yl)-2-morpholinooxazolo[4,5-b]pyridin-6-yl)picolinamide; |
| 95 | (R)-N-(5-(3-(cyclopropylmethoxy)pyrrolidin-1-yl)-2-morpholinooxazolo[4,5- |
| | b]pyridin-6-yl)-2-(2-methylpyridin-4-yl)oxazole-4-carboxamide; |
| 96 | 6′-amino-N-(2-morpholino-5-(piperidin-1-yl)oxazolo[4,5-b]pyridin-6-yl)-[2,3′- |
| | bipyridine]-6-carboxamide; |
| 97 | (R)-1-(6-(2-(2-methylpyridin-4-yl)oxazole-4-carboxamido)-2- |
| | morpholinooxazolo[4,5-b]pyridin-5-yl)pyrrolidin-3-yl glycinate; |
| 98 | 5-(4-aminopiperidin-1-yl)-N-(5-cyclopropyl-2-morpholinothiazolo[4,5-b]pyridin- |
| | 6-yl)pyrazolo[1,5-a]pyrimidine-3-carboxamide 2,2,2-trifluoroacetate; |
| 99 | 5-(4-aminopiperidin-1-yl)-N-(5-cyclopropyl-2-morpholinothiazolo[4,5-b]pyridin- |
| | 6-yl)pyrazolo[1,5-a]pyrimidine-3-carboxamide 2,2,2-trifluoroacetate; |
| 100 | (S)-N-(5-cyclopropyl-2-morpholinothiazolo[4,5-b]pyridin-6-yl)-5-(3- |
| | hydroxypiperidin-1-yl)pyrazolo[1,5-a]pyrimidine-3-carboxamide; |
| 101 | N-(5-cyclopropyl-2-morpholinothiazolo[4,5-b]pyridin-6-yl)-5-(4- |
| | hydroxypiperidin-1-yl)pyrazolo[1,5-a]pyrimidine-3-carboxamide; |
| 102 | 5-(3-aminopiperidin-1-yl)-N-(5-((R)-3-hydroxypyrrolidin-1-yl)-2- |
| | morpholinothiazolo[4,5-b]pyridin-6-yl)pyrazolo[1,5-a]pyrimidine-3-carboxamide |
| | hydrochloride; |
| 103 | (R)-N-(5-(3-hydroxypyrrolidin-1-yl)-2-morpholinothiazolo[4,5-b]pyridin-6- |
| | yl)pyrazolo[1,5-a]pyrimidine-3-carboxamide hydrochloride; |
| 104 | N-(2-morpholino-5-(piperidin-1-yl)thiazolo[4,5-b]pyridin-6-yl)pyrazolo[1,5- |
| | a]pyrimidine-3-carboxamide hydrochloride; |
| 105 | N-(5-cyclopropyl-2-morpholinothiazolo[4,5-b]pyridin-6-yl)pyrazolo[1,5- |
| | a]pyrimidine-3-carboxamide; |
| 106 | N-(5-cyclopropyl-2-morpholinothiazolo[4,5-b]pyridin-6-yl)pyrazolo[1,5- |
| | a]pyrimidine-3-carboxamide hydrochloride; or |
| 107 | N-(5-cyclopropyl-2-morpholinooxazolo[4,5-b]pyridin-6-yl)pyrazolo[1,5- |
| | a]pyrimidine-3-carboxamide; |

or a pharmaceutically acceptable salt or a stereoisomer thereof.

20. A pharmaceutical composition, comprising at least one compound according to any one of claims 1 to 19, or a pharmaceutically acceptable salt or a stereoisomer thereof and a pharmaceutically acceptable carrier, a pharmaceutically acceptable excipient or a pharmaceutically acceptable diluent.

21. The compound according to any one of claims 1 to 19, or a pharmaceutically acceptable salt or a stereoisomer thereof, for use as a medicament.

22. A method of treating an IRAK-4 mediated disorder or disease or condition in a subject comprising administering a compound according to any one of claims 1 to 19.

23. The method of claim 22, wherein the IRAK-4-mediated disorder or disease or condition is selected from the group consisting of cancer, an inflammatory disorder, an autoimmune disease, a metabolic disorder, a hereditary disorder, a hormone-related disease, immunodeficiency disorders, a condition associated with cell death, a destructive bone disorder, thrombin-induced platelet aggregation, liver disease and a cardiovascular disorder.

24. The method of claim 23, wherein the cancer is selected from a solid tumor, benign or malignant tumor, carcinoma of the brain, kidney, liver, stomach, vagina, ovaries, gastric tumors, breast, bladder colon, prostate, pancreas, lung, cervix, testis, skin, bone or thyroid; sarcoma, glioblastomas, neuroblastomas, multiple myeloma, gastrointestinal cancer, a tumor of the neck and head, an epidermal hyperproliferation, prostate hyperplasia, a neoplasia, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, papillary carcinoma, seminoma, melanoma; haematological malignancies selected from leukemia, acute myeloid leukemia (AML), chronic myeloid leukemia (CML), diffuse large B-cell lymphoma (DLBCL), activated B-cell-like DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, intravascular large B-cell lymphoma, plasmacytoma and multiple myeloma.

25. The method of claim 23, wherein the inflammatory disorder is selected from ocular allergy, conjunctivitis, keratoconjunctivitis sicca, vernal conjunctivitis, allergic rhinitis, autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, primary biliary cirrhosis, uveitis (anterior and posterior), Sjogren's syndrome, interstitial lung fibrosis, psoriatic arthritis, systemic juvenile idiopathic arthritis, nephritis, vasculitis, diverticulitis, interstitial cystitis, glomerulonephritis (e.g., including idiopathic ephritic syndrome or minimal change nephropathy), chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS) and osteoarthritis.

26. The compound according to any one of claims 1 to 19, or a pharmaceutically acceptable salt or a stereoisomer thereof, for use in treating a cancer, an inflammatory disorder, an autoimmune disease, metabolic disorder, a hereditary disorder, a hormone-related disease, immunodeficiency disorders, a condition associated with cell death, a destructive bone disorder, thrombin-induced platelet aggregation, liver disease and a cardiovascular disorder.

27. Use of the compound according to any one of claims 1 to 19, or a pharmaceutically acceptable salt or a stereoisomer thereof, in the manufacture of a medicament for the treatment of a cancer, an inflammatory disorder, an autoimmune disease, a metabolic disorder, a hereditary disorder, a hormone-related disease, immunodeficiency disorders, a condition associated with cell death, a destructive bone disorder, thrombin-induced platelet aggregation, liver disease and a cardiovascular disorder.
